(19) United States
(12) Patent Application Publication
(10) Pub. No.: US 2025/0324980 A1
(43) Pub. Date: Oct. 23, 2025

(54) COMPOSITIONS FOR DELIVERY OF AGENTS INTO PLANT CELLS

(71) Applicant: Ohio State Innovation Foundation, Columbus, OH (US)

(72) Inventors: Guoliang WANG, Columbus, OH (US); Dehua PEI, Columbus, OH (US); Jiyang WANG, Beijing (CN); Prabhat BHAT, Columbus, OH (US)

(21) Appl. No.: 18/862,643

(22) PCT Filed: May 4, 2023
(86) PCT No.: PCT/US2023/066615

§ 371 Date: Nov. 4, 2024

Related U.S. Application Data
(60) Provisional application No. 63/338,302, filed on May 4, 2022.

(51) Int. Cl. A01N 63/50; A01P 21/00; C07K 14/32; C07K 14/415

(52) U.S. Cl. CPC A01N 63/50; A01P 21/00; C07K 14/32; C07K 14/415; C07K 2319/03

(57) ABSTRACT

Described are peptides including a membrane translocation domain having one or more cell penetrating peptide motifs, and a cargo moiety linked to the membrane translocation domain, wherein the cargo moiety includes a plant bioactive moiety. The at least one cell penetrating peptide motif is from 3 to 10 amino acid residues in length and has at least three arginine and/or lysine residues; or the at least one cell penetrating peptide motif is from 3 to 10 amino acid residues in length and has at least two arginine and/or lysine residues and at least one other cell penetrating peptide motif is from 2 to 8 amino acid residues in length and has at least two hydrophobic residues. Also described are methods of delivering a cargo moiety into a plant cell comprising contacting the plant cell with the peptide as disclosed herein.

### CROSS-REFERENCE TO RELATED APPLICATIONS

The application claims the benefit of U.S. Provisional Application No. 63/338,302, filed May 4, 2022, which is hereby incorporated herein by reference in its entirety.

### REFERENCE TO SEQUENCE LISTING

The Sequence Listing submitted May 4, 2023, as a text filed named “103361-267WO1_ST26” created May 4, 2023, and having a file size of 295,920 bytes is hereby incorporated by reference pursuant to 37 C.F.R. § 1.52(e)(5).

### BACKGROUND

Although fertilizers and pesticides play a crucial role in modern agriculture, their adverse impact on the environment and animal/human health have recently inspired a rapid rise in organic farming. It is clear that organic agriculture with a ˜$80 billion market will become an essential, innovative farming system that balances sustainability with food/ecosystem security and human health benefits (Reganold and Wachter, 2016). A promising environment-friendly innovation is the use of (i) biological defense activators that improve plant immunity against pathogens and insects, and (ii) biostimulants that enhance plant growth and tolerance to abiotic stresses. Many of the plant defense activators and biostimulants now being used in organic farming are proteins/peptides that trigger the appropriate signal transduction pathways and thereby stimulate defense and/or growth. A formidable challenge in using these peptides/proteins, however, is their poor penetration efficiency in foliar applications and seed treatments (Nadendla, S. R., et al., Carbohyd Polym 199, 11-19 (2018)).

Cell penetrating peptides (CPPs) were first discovered in the early 1990s (Vives, E., et al., J Biol Chem 272, 16010-16017 (1997) and Derossi, D., et al., J Biol Chem 269, 10444-10450 (1994)). Since then, nearly 2000 CPPs have been reported, the vast majority of which are linear peptides. Despite much effort in academia and industry, drug delivery with linear CPPs has largely been unsuccessful because linear CPPs are proteolytically unstable and exhibit low cytosolic entry efficiencies as well as poor pharmacokinetics. An important advance resulted from cyclization of CPPs, as the cyclic variants are proteolytically stable and display 60-fold improved cytosolic entry efficiencies compared to their linear counterparts (Qian, Z. et al. Biochemistry 55, 2601-2612 (2016)). Further studies revealed that the robust activity of cyclic CPPs is due to their ability to escape the endosome through a previously unappreciated vesicular budding and collapse route (Sahni, A., et al., ACS Chem Biol 15, 2485-2492 (2020)). This understanding in turn led to the realization that CPPs must adopt appropriate 3-D structures to achieve the high cell-penetrating activity. Building on this insight, Bhat et al. recently engineered MTD4, a highly effective CPP, which is derived from the tenth human fibronectin type III (FN3) domain. Unlike cyclic CPPs that must be chemically synthesized before conjugation to a protein, MTD4 can be genetically fused with any peptide/protein cargo and produced recombinantly.

Studies on CPP-mediated protein delivery in plants were initiated 15 years ago. The first study showed that CPPs internalize into Nicotiana tabacum protoplasts, indicating that these peptides can enter plant cells by transfection (Mäe, M., et al. Biochimica et Biophysica Acta (BBA)-Biomembranes 1669, 101-107 (2005)). Subsequently, translocation of various CPPs in wheat immature embryos in the presence of a cell membrane-permeabilizing agent was also reported (Chugh, A., et al., FEBS J 275, 2403-2414 (2008)). Successful uptake of protein cargo by live microspore cells was also accomplished by utilizing a reversible disulfide bond between the R9 CPP and mCherry protein (Bilichak, A., et al., Front Plant Sci 6 (2015)). Recently, the penetration efficiency of 55 CPPs, most of them previously tested in animals, was assessed in dicot and monocot plants and some CPPs were found to enter plant cells (Numata, K., et al., SCI REP-UK 8 (2018)). An investigation into the delivery efficiency of two CPPs into rice calli revealed that a 5-day-old callus is better suited for CPP uptake than a 21-day-old callus (Guo, B., et al., PLOS ONE 14, e214033 (2019)). Although many CPPs have been tested in plants, there are no reports of the use of CPPs to deliver either defense- or growth-promoting proteins/peptides to crop plants.

The compositions and methods disclosed herein address these and other needs.

### SUMMARY

Disclosed herein are compounds, compositions, methods for making and using such compounds and compositions. In one aspect, disclose are peptides comprising a membrane translocation domain having one or more cell penetrating peptide motifs, and a cargo moiety linked to the membrane translocation domain, wherein the cargo moiety includes a plant bioactive moiety, where at least one cell penetrating peptide motif is from 3 to 10 amino acid residues in length and has at least three arginine and/or lysine residues; or where at least one cell penetrating peptide motif is from 3 to 10 amino acid residues in length and has at least two arginine and/or lysine residues and at least one other cell penetrating peptide motif is from 2 to 8 amino acid residues in length and has at least two hydrophobic residues. Also disclosed are methods of delivering a plant bioactive moiety into a plant cell comprising contacting the plant cell with the peptide as disclosed herein.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects and together with the description serve to explain the principles of the invention.

FIG. 1 shows structures of WT FN3 and MTD1-5. The FN3 structure was generated from the PDB file 1ttg and the structures of MTD1-5 were predicted by Phyre2. The inserted CPP motifs of MTD1-5 are highlighted in black.

FIGS. 2A and 2B. shows the expression and purification of MTD4. FIG. 2A is a FPLC chromatogram showing the elution of MTD4 from a Ni-NTA column (MTD4 elutes as a broad peak); FIG. 2B is a SDS-PAGE showing the expression level and different fractions during purification on a Ni-NTA column. L, molecular-weight markers; U, crude lysate of uninduced cells; I, crude lysate of IPTG-induced cells; CL, crude cell lysate after centrifugation; FT, flow-through fraction; 1-10, Ni-NTA column elution fractions (the intense band is MTD4).

FIGS. 3A-3B. show (3A) MTD4 structure. (3B) MTD4 protein permeability in N. benthamiana leaves. Ten μM rhodamine-labeled MTD4 (MTD4-Rh) or FN3 (FN3-Rh) were sprayed on N. benthamiana leaves 2 days after GFP infiltration. Two h after spraying, the sprayed leaf area was washed with H₂O₂ to remove any residual protein before measuring red (Rh) and green fluorescence (GFP) with a confocal microscope.

FIGS. 4A-4G. show enhanced permeability and disease resistance of HrpZ by MTD4 in tobacco and tomato. (4A) is an image showing cell death caused by MTD4-HrpZ treatment. Two ml of 10 μM MTD4-HrpZ, HrpZ, or MTD4 recombinant protein were placed on leaves of 6-week-old N. tabacum plants. The images shown were taken 24 h after the respective protein treatment. (4B) show images of reactive oxygen species (ROS) accumulation after MTD4-HrpZ treatment. The MTD4-HrpZ, HrpZ, or MTD4 (10 μM) was sprayed on 1-month-old N. tabacum leaves. ROS accumulation was detected by 3,3′-diaminobenzidine (DAB) staining. The images were taken 12 h post-treatment with the respective protein. (4C-4D) are graphs showing qRT-PCR analysis of the expression levels of (4C) NtHSR203 and (4D) NtCHN50 after the treatment with MTD4-HrpZ, HrpZ or MTD4 protein. The letters (a, b, c) indicate a significant difference (P<0.05, Dunnett's multiple range test). (4E) is a graph showing enhanced resistance in MTD4-HrpZ-treated plants. The MTD4-HrpZ, HrpZ, or MTD4 proteins (2 μM) were sprayed on N. tabacum leaves. Twelve h post-spraying, the leaves were pressure-inoculated with Pst DC3000 hrcC− at OD₆₀₀˜0.2. In planta bacterial numbers were assessed at 0 and 3 days post-inoculation and are shown here as colony-forming units (cfu). Data listed are mean values±standard errors; a and b indicate statistically significant differences (Duncan's multiple range test, P<0.05). (4F) are images showing disease symptoms of the MTD4-HrpZ, HrpZ or MTD4 treated tomatoes after B. cinerea inoculation. MTD4-HrpZ, HrpZ or MTD4 protein (5 μM) was sprayed on disinfected tobacco surface. The B. cinerea hyphal blocks were placed on a small wounded site of tomato fruits 24 h protein treatment. Photos were taken at 3 dpi. (4G) is a graph showing the diameter of the infection zones measured at 3 dpi.

FIG. 5. is an image showing cell death caused by MTD4-HrpZ in Arabidopsis post-spraying on leaves of 6-week-old plants. Images were taken 24 h post-treatment.

FIGS. 6A-6C are images showing that MTD4-HrpZ confers resistance to tomato bacterial speck pathogen. (6A) image after application of MTDA4, 1 μM, (6B) image after application of HrpZ, 1 μM, and (6C) image after application of MTD4-HrpZ, 1 μM.

FIGS. 7A-7D are images showing that MTD4-HrpZ confers resistance to tomato bacterial spot pathogen. (7A) image after application of MTD4; (7B-7D) are images after application of MTD4-HrpZ at different cell lysate dilutions: (7B) 3× dilution of cell lysates, (7C) 4× dilution of cell lysates, and (7D) 5× dilution of cell lysates.

FIG. 8 is an image showing that MTD4-HrpZ promotes tomato growth. Plants were sprayed twice with the 3 proteins and the data were obtained from one experiment with two replicates.

FIGS. 9A-9D are graphs showing that MTD4-HrpZ promotes tomato growth. (9A) is a graph of plant height up to the shoot tip for MTD4, HrpZ, and MTD4-HrpZ. (9B) is a graph of plant height up to the leaf tip for MTD4, HrpZ, and MTD4-HrpZ. (9C) is a graph of fresh weight (g) for MTD4, HrpZ, and MTD4-HrpZ. (9D) is a graph of dry weight (g) for MTD4, HrpZ, and MTD4-HrpZ. The letters (a, b, c) indicate a significant difference (P<0.05, Dunnett's multiple range test).

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

Throughout the present specification, the terms “about” and/or “approximately” may be used in conjunction with numerical values and/or ranges. The term “about” is understood to mean those values near to a recited value, as well as the recited value.

Throughout the present specification, numerical ranges are provided for certain quantities. It is to be understood that these ranges comprise all values and subranges therein. Thus, the range “from 50 to 80” includes all possible values therein (e.g., 50, 51, 52, 53, 54, 55, 56, etc.) and all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a polypeptide conjugate” refers to one or more polypeptide conjugates or at least one polypeptide conjugate. As such, the terms “a” (or “an”), “one or more” and “at least one” are used interchangeably herein. In addition, reference to “a polypeptide conjugate” by the indefinite article “a” or “an” does not exclude the possibility that more than one of the polypeptide conjugates is present, unless the context clearly requires that there is one and only one of the polypeptide conjugates.

As used herein, the term “adjacent” refers to two contiguous amino acids, which are connected by a covalent bond. “Adjacent” is also used interchangeably with “consecutive.”

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, “cell penetrating peptide” or “CPP” refers to any peptide including proteins (i.e., polypeptides) which is capable of penetrating a cell membrane. As used herein, “cyclic cell penetrating peptide” or “cCPP” refers to any cyclic peptide which is capable of penetrating a cell membrane.

A “foliar treatment” as used herein refers to a composition that is applied to the above ground parts or foliage of a plant or plant part and may have leaves, stems, flowers, branches, or any aerial plant part, for example, scion.

As used herein, “linker” or “L” refers to a moiety that covalently attaches two or more components of the polypeptide conjugates disclosed herein (e.g., a linker may covalently attach a CPP and a group that binds to a nucleic acid sequence by electrostatic interactions (i.e., P). In some embodiments, the linker can be natural or non-natural amino acid or polypeptide. In other embodiments, the linker is a synthetic compound containing two or more appropriate functional groups suitable to bind, e.g., the CPP and, independently, P. In some embodiments, the linker is about 3 to about 100 (e.g., about 3 to about 20) atoms in linear length (not counting the branched atoms or substituents). In some embodiments, the linker provides about 1 Å to about 400 Å in distance of the two groups to which it connects.

As used herein, “polypeptide” refers to a string of at least two amino acids attached to one another by a peptide bond. There is no upper limit to the number of amino acids that can be included in a polypeptide. Further, polypeptides may include non-natural amino acids, amino acid analogs, or other synthetic molecules that are capable of integrating into a polypeptide.

As used herein, a “monomer” refers to an amino acid residue in a polypeptide. In some embodiments, an amino acid monomer is divalent. In other embodiments, an amino acid monomer may be trivalent if the monomer is further substituted. For example, a cysteine monomer can independently form peptide bonds at the N and C termini, and also form a disulfide bond.

As used herein, an “amino acid-analog” or “analog” (e.g., “arginine-analog”, “lysine-analog” or “histidine-analog”) refers to a variant of an amino acid that retains at least one function of the amino acid, such as the ability to bind an oligonucleotide through electrostatic interactions. Such variants may have an elongated or shorter side chain (e.g., by one or more —CH₂— groups that retains the ability to bind an oligonucleotide through electrostatic interactions, or alternatively, the modification can improve the ability to bind an oligonucleotide through electrostatic interactions. For example, an arginine analog may include an additional methylene or ethylene between the backbone and guanidine/guanidinium group. Other examples include amino acids with one or more additional substituents (e.g., Me, Et, halogen, thiol, methoxy, ethoxy, C1-haloalkyl, C2-haloalkyl, amine, guanidine, etc). The amino acid-analog can be monovalent, divalent, or trivalent.

Throughout the present specification, peptides and amino acid monomers are depicted as charge neutral species. It is to be understood that such species may bear a positive or negative charge depending on the conditions. For example, at pH 7, the N-terminus of an amino acid is protonated and bears a positive charge (—NH₃⁺), and the C-terminus of an amino acid is deprotonated and bears a negative charge (—CO₂⁻). Similarly, the side chains of certain amino acids may bear a positive or negative charge.

Each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, alloisoleucine, arginine, asparagine, aspartic acid, cysteine, glutamine, glutamic acid, glycine, histidine, isoleucine, leucine, lysine, methionine, naphthylalanine, phenylalanine, proline, pyroglutamic acid, serine, threonine, tryptophan, tyrosine, valine, a derivative, or combinations thereof. Analogs of amino acids encompass that have a structural similar but not identical to an amino acid, e.g., due to a modification to the side chain or backbone on said amino acid. Such modifications may increase the hydrophobicity of the side chain, including elongation of the side chain by one or more hydrocarbons, or increasing the the solvent accessible surface area (SASA as described herein) of an amino acid having an aromatic ring on its side chain, e.g., by conjugating a second aromatic ring or increasing the size of the aromatic ring. Derivatives of amino acids encompass natural and non-natural amino acids that have been modified (e.g., by substitution) to include a hydrophobic group as described herein. For example, a derivative of lysine includes lysine whose side chain has been substituted with alkylcarboxamidyl. These, and others, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| AIlosoleucine | AIle | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Cyclohexylalanine | Cha | Cha |
| 2,3-diaminopropionic acid | Dap | Dap |
| 4-fluorophenylalanine | Fpa (Σ) | Pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (1) |
| Lysine | Lys (K) | lys (k) |
| Methionine | Met (M) | met (m) |
| Naphthylalanine | Nal (Φ) | nal (ϕ) |
| Norleucine | Nle (Ω) | nle |
| Phenylalanine | Phe (F) | phe (f) |
| Phenylglycine | Phg (Ψ) | phg |
| 4-(phosphonodifluoromethyl)phenylalanine | F₂Pmp (A) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| 3-(3-benzothienyl)-alanine | Bta | Bta |
| 4-fluorophenylalanine | Fpa or L-Fpa | fpa or D-Fpa |
| 1-naphthylalanine | L-1-Nal or 1-Nal | D-1-Nal or 1-nal |
| 2-naphthylalanine | L-2-Nal or 2-Nal | D-2-Nal or 2-nal |
| 2-pyridylalanine | L-2-Pya or 2-Pya | D-2-Pya or 2-pya |
| 4-pyridylalanine | L-4-Pya or 4-Pya | D-4-Pya or 4-pya |

*single letter abbreviations: when shown in capital letters herein it indicates the L-amino acid form, when shown in lower case herein it indicates the D-amino acid form.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, in-hexyl, n-heptyl, n-octyl, in-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, pentylene, and the like. Unless stated otherwise specifically in the specification, an alkylene chain can be optionally substituted as described herein.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethenylene (—CH═CH—), propenylene, butenylene, and the like. Unless stated otherwise specifically in the specification, an alkenylene chain can be optionally substituted.

“Alkynyl” or “alkynyl group” refers to a straight or branched hydrocarbon chain radical having from two to twelve carbon atoms and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to forty carbon atoms, and having one or more carbon-carbon triple bonds. Non-limiting examples of C₂-C₄₀ alkynylene include ethynylene (—C≡C—), propargylene and the like. Unless stated otherwise specifically in the specification, an alkynylene chain can be optionally substituted.

“Aryl” refers to a hydrocarbon ring system comprising hydrogen, 6 to 40 carbon atoms and at least one aromatic ring. For purposes of this disclosure, the aryl can be a monovalent or a divalent radical (not counting substituents), which can be a monocyclic, bicyclic, tricyclic or tetracyclic ring system, and which can include fused or bridged ring systems. Aryl radicals include, but are not limited to, radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl radical can be divalent when used as a linker or as a part of a linker. Unless stated otherwise specifically in the specification, an aryl group can be optionally substituted.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl and rings that are fully unsaturated, partially unsaturated, and fully saturated. In some embodiments, the carbocyclyl can be divalent when used as a linker or as a part of a linker. Unless stated otherwise specifically in the specification, a carbocyclyl group can be optionally substituted.

“Cycloalkyl” refers to a stable non-aromatic monocyclic or polycyclic fully saturated hydrocarbon radical having from 3 to 40 carbon atoms and at least one ring, wherein the ring consists solely of carbon and hydrogen atoms, which can include fused or bridged ring systems. For purposes of this disclosure, the cycloalkyl can be a monovalent or a divalent radical (not counting substituents). Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. In some embodiments, the cycloalkyl radical can be divalent when used as a linker or as a part of a linker. Unless otherwise stated specifically in the specification, a cycloalkyl group can be optionally substituted.

“Cycloalkenyl” refers to a stable non-aromatic monocyclic or polycyclic hydrocarbon radical having from 3 to 40 carbon atoms, at least one ring having, and one or more carbon-carbon double bonds, wherein the ring consists solely of carbon and hydrogen atoms, which can include fused or bridged ring systems. For purposes of this invention, the cycloalkenyl can be a monovalent or a divalent radical (not counting substituents). Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. In some embodiments, the cycloalkenyl radical can be divalent when used as a linker or as a part of a linker. Unless otherwise stated specifically in the specification, a cycloalkenyl group can be optionally substituted.

“Cycloalkynyl” refers to a stable non-aromatic monocyclic or polycyclic hydrocarbon radical having from 3 to 40 carbon atoms, at least one ring, and one or more carbon-carbon triple bonds, wherein the ring consists solely of carbon and hydrogen atoms, which can include fused or bridged ring systems. For purposes of this invention, the cycloalkynyl can be a monovalent or a divalent radical (not counting substituents). Monocyclic cycloalkynyl radicals include, for example, cycloheptynyl, cyclooctynyl, and the like. In some embodiments, the cycloalkynyl radical can be divalent when used as a linker or as a part of a linker. Unless otherwise stated specifically in the specification, a cycloalkynyl group can be optionally substituted.

“Heterocyclyl,” “heterocyclic ring” or “heterocycle” refers to a stable 3- to 20-membered aromatic ring radical which consists of two to twelve carbon atoms and from one to six heteroatoms selected from the group consisting of nitrogen, oxygen and sulfur. For purposes of this invention, the heterocyclyl radical can be a monovalent or a divalent radical (not counting substituents). Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl radical can be divalent when used as a linker or as a part of a linker. Unless stated otherwise specifically in the specification, a heterocyclyl group can be optionally substituted.

“Heteroaryl” refers to a 5- to 20-membered ring system radical comprising hydrogen atoms, one to fourteen carbon atoms, one to six heteroatoms selected from the group consisting of nitrogen, oxygen and sulfur, and at least one aromatic ring. For purposes of this invention, the heteroaryl radical can be a monovalent or a divalent radical (not counting substituents) and can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). In some embodiments, the heteroaryl radical can be divalent when used as a linker or as a part of a linker. Unless stated otherwise specifically in the specification, a heteroaryl group can be optionally substituted.

The term “ether” used herein refers to a straight or branched divalent radical moiety —[(CH₂)ₘ—O—(CH₂)ₙ]_{z}— wherein each of m, n, and z are independently selected from 1 to 40. Examples include, but are not limited to, polyethylene glycol. Unless stated otherwise specifically in the specification, the ether can be optionally substituted.

The term “substituted” used herein means any of the above groups (i.e., alkylene, alkenylene, alkynylene, aryl, carbocyclyl, cycloalkyl, cycloalkenyl, cycloalkynyl, heterocyclyl, heteroaryl, and/or ether) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced

with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents. Further, those skilled in the art will recognize that “substituted” also encompasses instances in which one or more atoms on any of the above groups are replaced by a substituent listed in this paragraph, and the substituent forms a covalent bond with the CPP, P, or L. For example, in certain embodiments, any of the above groups can be substituted at a first position with a carboxylic acid (i.e., —C(═O)OH) which forms an amide bond with a lysine in the CPP, or a group can be substituted at a second position with a thiol group which forms a disulfide bond with a cysteine (or amino acid analog having a thiol group).

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an amino acid residue in a peptide or protein refers to one or more —OC(O)CH(R)NH— units in the peptide or protein.

As used herein, the symbol

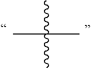

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

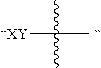

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

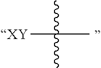

infers that when R³ is “XY”, the point of attachment bond is the same bond as the bond by which R³ is depicted as being bonded to CH₃.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the compounds and compositions disclosed herein include all such possible diastereomers as well as their racemic mixtures, their substantially pure resolved enantiomers, all possible geometric isomers, and pharmaceutically acceptable salts thereof Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds disclosed herein include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds and prodrugs thereof can generally be prepared by carrying out the procedures below, by substituting a readily available isotopically labeled reagent for a non-isotopically labeled reagent.

Disclosed are the components to be used to prepare the compositions disclosed herein as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions disclosed herein. Thus, if there are a variety of additional steps that can be performed it is understood that each of these additional steps can be performed with any specific embodiment or combination of embodiments of the methods disclosed herein.

The term “contacting” as used herein refers to bringing a disclosed compound and a target (e.g., a cell, target receptor, transcription factor, or other biological entity) together in such a manner that the compound can affect the activity of the target either directly, i.e., by interacting with the target itself, or indirectly, i.e., by interacting with another molecule, co-factor, factor, or protein on which the activity of the target is dependent.

As used herein, the terms “effective amount” and “amount effective” refer to an amount that is sufficient to achieve the desired result.

Reference will now be made in detail to specific aspects of the disclosed materials, compounds, compositions, articles, and methods, examples of which are illustrated in the accompanying Examples and Figures.

### Compounds

Disclosed are cell-permeable peptides and compositions comprising them, which can provide a general vehicle for cytosolic delivery of potentially any peptide or protein cargo as well as other biomolecules including oligonucleotides. The disclosed peptides can have greater cytosolic delivery efficiency and in vivo stability over simple cell permeable peptides.

In a specific aspect, disclosed herein are peptides comprising: a membrane translocation domain having one or more cell penetrating peptide motifs, and a cargo moiety linked to the membrane translocation domain, wherein the cargo moiety includes a plant bioactive moiety, where at least one of the cell penetrating peptide motifs is from 3 to 10 amino acid residues in length and has at least three arginine and/or lysine residues.

Unlike methods where a CPP motif is inserted into each target protein, the disclosed compounds, compositions and methods involve an engineered membrane translocation domain that can be genetically or synthetically fused to any target cargo of interest. Another strategy disclosed herein involves splitting a CPP motif into two halves and inserting them into two different regions of the membrane translocation domain, resulting in greatly improved cytosolic delivery efficiencies.

In some embodiments, the compounds described herein can be used as plant activators. The term “plant activator” refers to a compound that activates a natural defense mechanism in a host plant, such as systemic acquired resistance (SAR), or hypersensitive response. The compositions can be used as a plant activators for either healthy and unhealthy plants, or plants in both healthy and unhealthy environments.

In some embodiments, the compounds described herein can be used as plant stimulants. The term “plant stimulant,” as used herein refers to a compound or compositions applied to plants under conditions that enhance nutrition efficiency, stress tolerance, and/or crop quality traits, regardless of its nutrition content. Particularly, plant stimulants are used in the cultivation of plants in order to improve the growth and development processes. The impact of stimulants on plants is not due to direct participation in the regulation of life processes, but the effect on metabolism in the broad sense of this word. They can stimulate the synthesis of natural hormones, and sometimes increase their activity, can improve intake of minerals from the soil, regulate the growth of roots. In addition, they can cause the increase of the resistance to adverse conditions (biotic or abiotic). The use of stimulants in the cultivation of plants increases the yields, often while increasing their quality at the same time. Stimulants can enhance life processes occurring in plants without changing plants natural behavior. The compounds and/or compositions described herein can be plant stimulants and therefore can be used as plant growth regulators, plant metabolic processes regulators, plant physiological processes regulators, a substance that prevents against the effects of biotic or abiotic stress in a plant, and/or a substance that provides multiple disease resistance to a plant. The compositions can be used as a plant stimulant for either healthy and unhealthy plants, or plants in both healthy and unhealthy environments.

### Membrane Translocation Domain

The membrane translocation domain portion of the disclosed peptides can be any membrane translocation domain, a peptide sequence that may traverse a lipid bilayer, that has been modified to contain at least one cell penetrating motifs as described herein. In a preferred example, there are two or three cell penetrating motifs in the membrane translocation domains. For example, at least one cell penetrating peptide motif can be from 3 to 10 amino acid residues in length and have at least three arginine and/or lysine residues, e.g., 4, 5, or 6 arginines and/or lysine residues. Alternatively, at least one cell penetrating peptide motif can be from 3 to 10 amino acid residues in length and have at least two arginine and/or lysine residues and at least one other cell penetrating peptide motif can be from 2 to 8 amino acid residues in length and have at least two hydrophobic residues. When there are two or more cell penetrating peptide motifs, there can be two or more arginine residues and/or lysine residues in a 3 to 10 amino acid span and another cell penetrating peptide motif where there are two or more hydrophobic residues within a 2 to 8 amino acid span. The cell penetrating peptide motifs can be anywhere in the membrane translocation domain.

In some embodiments, the membrane translocation domain can be a plant membrane translocation domain. In some examples, the membrane translocation domain can be a human membrane translocation domain, such as fibronectin type III. In a specific example, the membrane translocation domain has at least 90%, at least 95%, or at least 97% sequence similarity with SEQ. ID. NO.:118. In other examples, the membrane translocation domain is human fibronectin type III having BC, DE, CD, and FG loops and the cell penetrating peptide motif is in one or more of the BC, DE, CD, or FG loops, e.g., the cell penetrating peptide motif is in two of the BC, DE, CD, or FG loops, in particular the BC and FG loops. These loops can be defined as having the following sequences BC=AVTVR (SEQ ID NO:31); CD=GGNSPVQ (SEQ ID NO:32); DE=PGSK (SEQ ID NO:33); FG=GRGDSPAS (SEQ ID NO:34).

In other examples, the membrane translocation domain can be any stably folded protein, which can preferably be efficiently expressed in bacteria. Some additional examples of membrane translocation domains are the nanobody scaffold, DARPin scaffold, and CTPR protein (the consensus tetratricopeptide repeat; Acc. Chem. Res. 2021, 54, 4166-4177).

### Cell Penetrating Peptide Motif

The cell penetrating peptide (CPP) motif can comprises at least 2 amino acids, at least 3 amino acids, at least 4 amino acids, or at least 6 amino acids, more specifically from 3 to 8, from 3 to 6, from 4 to 8, from 4 to 6, or from 6 to 8 amino acids. In most examples, the CPP motif is substituted into the membrane translocation domain such that the resulting peptide has the same number of amino acids as in native membrane translocation domain.

In some examples, at least two, three, four, five, six, or seven amino acids of the CPP motif are adjacent arginine residues. In a preferred, example there are three, four, or five adjacent arginine residues in a CPP motif. In other examples, the arginine residues are not adjacent in the CPP motif. Each amino acid in the CPP motif can independently be a natural or non-natural amino acid. When such adjacent arginine or lysine residues are the CPP motif, then there need not be any additional CPP motifs, e.g., those with hydrophobic residues, though such a hydrophobic CPP motif can still be used. When the CPP motif contains two argine residues, then it is preferred that there be another CPP motif with at least two hydrophobic residues within 2 to 8 amino acids.

In other examples, at least one, at least, two, at least three, or more amino acids of the CPP motif are hydrophobic amino acids, i.e., have hydrophobic side chains. In some examples, the amino acids having hydrophobic side chains are independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, cyclohexylalanine, norleucine, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, tert-leucine, or nicotinoyl lysine, each of which is optionally substituted with one or more substituents. In particular examples, each amino acid having a hydrophobic side chain is independently an amino acid having an aromatic side chain. In some embodiments, the amino acid having an aromatic side chain is 3-benzothienyl-L-alanine, naphthylalanine, phenylglycine, homophenylalanine, phenylalanine, tryptophan, or tyrosine, each of which is optionally substituted with one or more substituents. Thus, in some examples, the amino acids having hydrophobic side chains are phenylalanine, naphthylalanine, tryptophan, or an analog or derivative thereof naphthylalanine or tryptophan, or analogues or derivatives thereof. In other examples, the CPP motif further comprises at least one phenylalanine, phenylglycine, or histidine, or analogues or derivatives thereof.

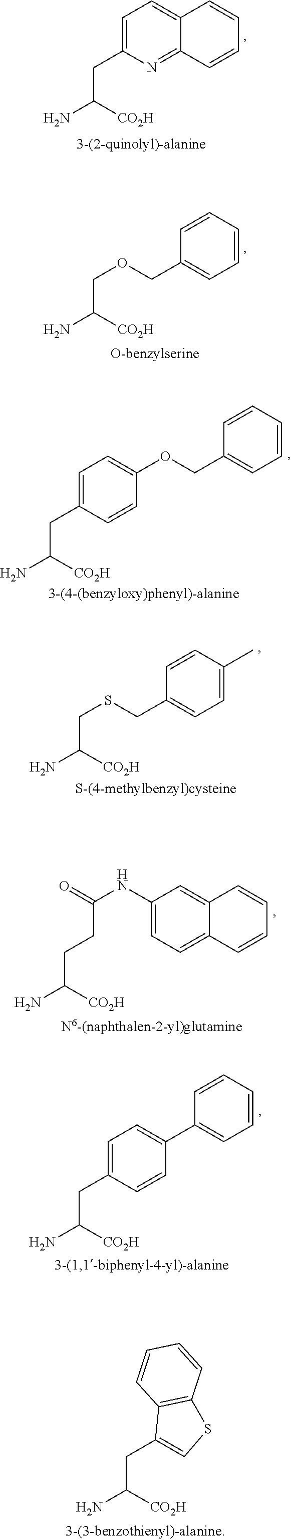

In some examples, the CPP motif can include any combination of at least three adjacent arginines and either at least two amino acids have a hydrophobic side chain selected from an aryl or heteroaryl, wherein the aryl and heteroaryl are optionally substituted, with a total number of amino acids in the CPP motif in the range of from 5 to about 8 amino acids.

In some examples, the membrane translocation domain is human fibronectin type III having BC, DE, CD, and FG loops and the CPP is in one or more of the BC, DE, CD, or FG loops. For example, the CPP motif is in two of the BC, DE, CD, or FG loops. In a specific example, the CPP motif is in the BC and either the DE, CD and FG loops, preferably in the BC and FG loops.

Where there are two or more CPP motifs, one CPP motif can be the 3 to 10 amino acid segment with at least two arginine and/or lysine residues and the other can be a 2 to 8 amino acid segment with at least two hydrophobic residues. For example, the membrane translocation domain can have two or more CPPs and at least one of the motifs is from 2 to 8 amino acid residues and has at least two hydrophobic amino acid residues.

In an example of this, the membrane translocation domain can be human fibronectin type III having BC, DE, CD, and FG loops, and the CPP motifs can be in the BC loop and have from 2 to 8 amino acid residues and has at least two hydrophobic amino acid residues and a CPP motif can be in the FG loop and have from 3 to 10 amino acid residues and has at least three adjacent arginine and/or lysine residues. Alternatively, the CPP motifs can be in the FG loop and have from 2 to 8 amino acid residues and has at least two hydrophobic amino acid residues and a CPP motif can be in the BC loop and have from 3 to 10 amino acid residues and has at least three adjacent arginine and/or lysine residues.

When the CPP motif contains the from 2 to 8 amino acid residues and has at least two hydrophobic amino acid residues, it can be WW, FF, WF, FW, WWW, FFF, WFW, FWF, WWF, WFF, FWW, FFW, WYW, WWH, YWW, or WYH. It is preferable that this CPP motif be in the BC loop. It is further preferable that this CPP motif be WW, FW, WF, WYW, WWW, WWH, YWW, WYH or YWH.

The CPP motif with 3 to 10 amino acid residues and has at least three adjacent arginine and/or lysine residues can contain RRR, RRRR, RRRRR. It can also be any combination of arginine and lysine residues. When this CPP motif is in the FG loop I can be 3-10 residues in length and of any combinations of Arg and Lys (and occasionally other non-acidic residues). The CPP motif (e.g., WWWRRRR) may be alternatively split, so that some of the Arg/Lys residues are moved from the FG loop into the BC loop (e.g., WWWR . . . RRR, WWWRR . . . RR, WWWRRRR . . . , etc.); The CPP motif (e.g., WWWRRRR) may be alternatively split, so that some of the hydrophobic residues are moved from the BC loop to the FG loop (e.g., WW . . . WRRR, W . . . WWRRRR, . . . WWWRRRR, etc.). The CPP motif (e.g., WWWRRRR) can be alternatively split, so that either BC or FG loop contains a combination of hydrophobic and positively charged residues (e.g., WWR . . . WRRR, WWRR . . . WRR, WWRR . . . RRW, RRW . . . WWRR, etc.).

In specific examples, the CPP motif comprises SEQ. ID. NOS.: 104, 105, 11, 112, 113, 114, 115, 116, or 117.

In some examples, the CPP motif can be or comprise any of the sequences listed in Table 2. In some examples, the cell penetrating peptide can be or comprise the reverse of any of the sequences listed in Table 2.

TABLE 2
CPP motif sequences

| | | #R |
|---|---|---|
| SEQ ID NO | CPP sequence | residues |
| SEQ ID NO: 1 | FΦRRR | 3 |
| SEQ ID NO: 2 | FΦRRRC | 3 |
| SEQ ID NO: 3 | FΦRRRU | 3 |
| SEQ ID NO: 4 | RRRΦF | 3 |
| SEQ ID NO: 5 | RRRRΦF | 4 |
| SEQ ID NO: 6 | FΦRRRR | 4 |
| SEQ ID NO: 7 | FϕrRrR | 3 |
| SEQ ID NO: 8 | FϕrRrR | 3 |
| SEQ ID NO: 9 | FΦRRRR | 4 |
| SEQ ID NO: 10 | fΦRrRr | 4 |
| SEQ ID NO: 11 | RRFRΦR | 4 |
| SEQ ID NO: 12 | FRRRRΦ | 4 |
| SEQ ID NO: 13 | FRFRΦR | 4 |
| SEQ ID NO: 14 | RRΦFRR | 4 |
| SEQ ID NO: 15 | CRRRRFW | 4 |
| SEQ ID NO: 16 | FfΦRrRr | 4 |
| SEQ ID NO: 17 | FFΦRRRR | 4 |
| SEQ ID NO: 18 | RFRFRΦR | 4 |
| SEQ ID NO: 19 | URRRRFW | 4 |
| SEQ ID NO: 20 | CRRRRFW | 4 |
| SEQ ID NO: 21 | FΦRRRRQK | 4 |
| SEQ ID NO: 22 | FΦRRRRQC | 4 |
| SEQ ID NO: 23 | FΦRrRrR | 5 |
| SEQ ID NO: 24 | FΦRRRRR | 5 |
| SEQ ID NO: 25 | RRRRΦFDΩC | 4 |
| SEQ ID NO: 26 | FΦRRR | 3 |
| SEQ ID NO: 27 | FWRRR | 3 |
| SEQ ID NO: 28 | RRRΦF | 3 |
| SEQ ID NO: 29 | RRRWF | 3 |
| SEQ ID NO: 30 | FΦRRRR | 4 |
| SEQ ID NO: 31 | FFRRR | 3 |
| SEQ ID NO: 32 | FFrRr | 3 |
| SEQ ID NO: 33 | FFRrR | 3 |
| SEQ ID NO: 34 | FRFRR | 3 |
| SEQ ID NO: 35 | FRRFR | 3 |
| SEQ ID NO: 36 | FRRRF | 3 |
| SEQ ID NO: 37 | GΦRRR | 3 |
| SEQ ID NO: 38 | FFFRA | 1 |
| SEQ ID NO: 39 | FFFRR | 2 |
| SEQ ID NO: 40 | FFRRRR | 4 |
| SEQ ID NO: 41 | FRRFRR | 4 |
| SEQ ID NO: 42 | FRRRFR | 5 |
| SEQ ID NO: 43 | RFFRRR | 4 |
| SEQ ID NO: 44 | RFRRFR | 4 |
| SEQ ID NO: 45 | FRFRRR | 4 |
| SEQ ID NO: 46 | FFFRRR | 3 |
| SEQ ID NO: 47 | FFRRRF | 3 |
| SEQ ID NO: 48 | FRFFRR | 3 |
| SEQ ID NO: 49 | RRFFFR | 3 |
| SEQ ID NO: 50 | FFRFRR | 3 |
| SEQ ID NO: 51 | FFRRFR | 3 |
| SEQ ID NO: 52 | FRRFFR | 3 |
| SEQ ID NO: 53 | FRRFRF | 3 |
| SEQ ID NO: 54 | FRFRFR | 3 |
| SEQ ID NO: 55 | RFFRFR | 3 |
| SEQ ID NO: 56 | GΦRRRR | 4 |
| SEQ ID NO: 57 | FFFRRRR | 4 |
| SEQ ID NO: 58 | RFFRRRR | 5 |
| SEQ ID NO: 59 | RRFFRRR | 5 |
| SEQ ID NO: 60 | RFFFRRR | 4 |
| SEQ ID NO: 61 | RRFFFRR | 4 |
| SEQ ID NO: 62 | FFRRFRR | 4 |
| SEQ ID NO: 63 | FFRRRRF | 4 |
| SEQ ID NO: 64 | FRRFFRR | 4 |
| SEQ ID NO: 65 | FFFRRRRR | 5 |
| SEQ ID NO: 66 | FFFRRRRRR | 6 |
| SEQ ID NO: 67 | FΦRrRr | 4 |
| SEQ ID NO: 68 | XXRRRR | 4 |
| SEQ ID NO: 69 | FfFRrR | 3 |
| SEQ ID NO: 70 | fFfrRr | 2 |
| SEQ ID NO: 71 | fFfRrR | 3 |
| SEQ ID NO: 72 | FfFrRr | 3 |
| SEQ ID NO: 73 | fFφrRr | 3 |
| SEQ ID NO: 74 | fΦfrRr | 3 |
| SEQ ID NO: 75 | φFfrRr | 3 |
| SEQ ID NO: 76 | FΦrRr | 3 |
| SEQ ID NO: 77 | fΦrRr | 3 |
| SEQ ID NO: 78 | Ac-Lys-fFRrRrD | 4 |
| SEQ ID NO: 79 | Ac-Dap-fFRrRrD | 4 |
| SEQ ID NO: 80 | Pip-Nal-Arg-Glu-arg-arg-glu | 3 |
| SEQ ID NO: 81 | Pip-Nal-Arg-Arg-arg-arg-glu | 3 |
| SEQ ID NO: 82 | Pip-Nal-Nal-Arg-arg-arg-glu | 3 |
| SEQ ID NO: 83 | Pip-Nal-Nal-Arg-arg-arg-Glu | 3 |
| SEQ ID NO: 84 | Pip-Nal-Phe-Arg-arg-arg-glu | 3 |
| SEQ ID NO: 85 | Pip-Nal-Phe-Arg-arg-arg-Glu | 3 |
| SEQ ID NO: 86 | Pip-Nal-phe-Arg-arg-arg-glu | 3 |
| SEQ ID NO: 87 | Pip-Nal-phe-Arg-arg-arg-Glu | 3 |
| SEQ ID NO: 88 | Pip-Nal-nal-Arg-arg-arg-Glu | 3 |
| SEQ ID NO: 89 | Pip-Nal-nal-Arg-arg-arg-glu | 3 |
| SEQ ID NO: 90 | [P-Cha-r-Cha-r-Cha-r-Cha-r- | 4 |
| | G]^{d} | |
| SEQ ID NO: 91 | Tm(SvP-F₂Pmp-H)-Dap-(FΦRRRR- | 4 |
| | Dap)]^{f} | |
| SEQ ID NO: 92 | [Tm(a-Sar-D-pThr-Pip-ΦRAa)- | 5 |
| | Dap-(FΦRRRR-Dap)]^{f} | |
| SEQ ID NO: 93 | [CRRSRRGCGRRSRRCG]^{g} | 8 |
| SEQ ID NO: 94 | [K(Dod)RRRR] | 4 |
| SEQ ID NO: 95 | LKKLCKLLKKLCKLAG | 0 |
| SEQ ID NO: 96 | RRRR-[KRRRE]^{c} | 7 |
| SEQ ID NO: 97 | RRR-[KRRRRE]^{c} | 7 |
| SEQ ID NO: 98 | RR-[KRRRRRE]^{c} | 7 |
| SEQ ID NO: 99 | R-[KRRRRRRE]^{c} | 7 |
| SEQ ID NO: 100 | [CRCRCRCR] | 4 |
| SEQ ID NO: 101 | [Pra-LRKRLRKFRN-AzK]^{h} | |
| SEQ ID NO: 102 | T-Dap-[Dap-Dap-f-L-Dap-Dap- | |
| | T] | |
| SEQ ID NO: 103 | T-Agp-[Dap-Agp-f-L-Agp-Agp- | |
| | T] | |

Φ=L-naphthylalanine; ϕ=D-naphthylalanine; Ω=L-norleucine; r=D-arginine; F=L-phenylalanine; f=D-phenylalanine; q=D-glutamine, X=L-4-fluorophenylalanine; Dap=L-2,3-diaminopropionic acid; Sar, sarcosine, F₂Pmp, L-difluorophosphonomethyl phenylalanine; Dod, dodecanoyl; Pra, L-propargylglycine, AzK, L-6-Azido-2-amino-hexanoic; Agp, L-2-amino-3-guanidinylpropionic acid; ^{b}Cyclization between Pim and Nlys; ^{c}Cyclizaton between Lys and Glu; ^{d}Macrocyclization by multicomponent reaction with aziridine aldehyde and isocyanide; ^{e}Cyclization between the main-chain of Gln residue; ^{f}N-terminal amine and side chains of two Dap residues bicyclized with Tm; ^{g}Three Cys side chains bicyclized with tris(bromomethyl)benzene; ^{h}Cyclization by the click reaction between Pra and Azk.

The chirality of the amino acids can be selected to improve cytosolic uptake efficiency. In some embodiments, at least two of the amino acids have the opposite chirality. In some embodiments, the at least two amino acids having the opposite chirality can be adjacent to each other. In some embodiments, at least three amino acids have alternating stereochemistry relative to each other. In some embodiments, the at least three amino acids having the alternating chirality relative to each other can be adjacent to each other. In some embodiments, at least two of the amino acids have the same chirality. In some embodiments, the at least two amino acids having the same chirality can be adjacent to each other. In some embodiments, at least two amino acids have the same chirality and at least two amino acids have the opposite chirality. In some embodiments, the at least two amino acids having the opposite chirality can be adjacent to the at least two amino acids having the same chirality. Accordingly, in some embodiments, adjacent amino acids in the cCPP can have any of the following sequences: D-L; L-D; D-L-L-D; L-D-D-L; L-D-L-L-D; D-L-D-D-L; D-L-L-D-L; or L-D-D-L-D.

### Cargo Moiety

The cargo moiety can be linked to the membrane translocation domain. The cargo moiety can be linked to an amino group (e.g., N-terminus), a carboxylate group (e.g., C-terminus), or a side chain of one or more amino acids in the in the membrane translocation domain.

When the cargo moiety is attached to the side chain of an amino acid in the membrane translocation domain, the membrane translocation domain includes an amino acid having a side chain with a suitable functional group to form a covalent bond (conjugation) with the cargo, or a side chain which may be modified to provide a suitable functional group (e.g., via conjugation of a linker) that forms a covalent bond with the cargo. In some embodiments, the amino acid on membrane translocation domain which has a side chain suitable conjugation of the cargo is a cysteine residue, glutamic acid residue, an aspartic acid residue, a lysine residue, or a 2,3-diaminopropionic acid residue. In such embodiments, the cargo may be directly conjugated to the side chain of the amino acid (e.g., by forming a disulfide bond with a cysteine residue or an amide bond with a glutamic acid residue or a 2,3-diaminopropionic acid residue) or the cargo may be conjugated to the amino acid side chain through a linker (e.g., PEG).

In some embodiments, the cargo moiety can include a plant bioactive moiety. In some embodiments, the cargo moiety can further include any cargo of interest, for example a linker moiety, a detectable moiety, or any combination thereof. In some examples, the cargo moiety can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-β-naphthylalanine; L-pipecolic acid (Pip); sarcosine; trimesic acid (Tm); 7-amino-4-methylcoumarin (Amc); fluorescein isothiocyanate (FITC); L-2-naphthylalanine; norleucine; 2-aminobutyric acid; Rhodamine B (Rho); Dexamethasone (DEX); or combinations thereof.

### Plant Bioactive Moiety

The cargo moiety can include a plant bioactive moiety. In some embodiments, a detectable moiety can be linked to a plant bioactive moiety. The plant bioactive moiety can be attached to the cell penetrating peptide moiety at the amino group, the carboxylate group, or the side chain of any of the amino acids of the cell penetrating peptide moiety (e.g., at the amino group, the carboxylate group, or the side chain or any of amino acid of the CPP). In some examples, the plant bioactive moiety can be attached to the detectable moiety.

The term “plant bioactive moiety” refers to a compound that activates a natural defense mechanism in a host plant, such as systemic acquired resistance (SAR), or hypersensitive response, or a compound that enhance nutrition efficiency, stress tolerance, and/or crop quality traits, regardless of its nutrition content.

Non-limiting examples of a plant bioactive moiety can include, but is not limited to, synthetically derived or naturally occurring flagellins and flagellin-associated polypeptides (including those conserved among the Bacillus genera), thionins, harpin protein or polypeptide or harpin-like polypeptide, elongation factor Tu (EF-Tu), phytosulfokine (PSKα), root hair promoting polypeptide (RHPP), hypersensitive response elicitor proteins or polypeptides, antifungal peptides, insect toxin peptides, antifreezing proteins, heat tolerance proteins, drought tolerance proteins, vitamin biosynthesis enzymes, bioherbicide peptides, or maize mitochondrial mutant protein, or any combination thereof. Suitable synthetically derived or naturally occurring flagellins and flagellin-associated polypeptides (including those conserved among the Bacillus genera), thionins, harpin protein or polypeptides or harpin-like polypeptide, elongation factor Tu (EF-Tu), phytosulfokine (PSKα), root hair promoting polypeptide (RHPP), and/or hypersensitive response elicitor proteins or polypeptides described herein, or described in International Application Publication No. WO 2019/018768, WO 2010/019442, WO 1998/054214, WO 2001/098501, and WO 2013/102189, each of which is hereby incorporated by reference in its entirety. The plant bioactive moiety can be selected for their distinct modes of action and can be used individually or in combination with other polypeptides to accommodate the specific agricultural needs. They can be used in the place of or in addition to commercially available agrochemicals, biostimulants, supplemental bioactives, pesticidal compounds, or any combination thereof.

Flagellins and flagellin-associated polypeptides derived from those flagellins have been reported primarily to have functional roles in innate immune responses in plants. These polypeptides are derived from highly conserved domains of eubacterial flagellin. Flagellin is the main building block of the bacterial flagellum. The flagellin protein subunit building up the filament of bacterial flagellum can act as a potent elicitor in cells to mount defense-related responses in various plant species.

“Flagellin” is a globular protein that arranges itself in a hollow cylinder to form the filament in a bacterial flagellum. Flagellin is the principal substituent of bacterial flagellum, and is present in flagellated bacteria. Plants can perceive, combat infection and mount defense signaling against bacterial microbes through the recognition of conserved epitopes, such as the stretch of 22 amino acids (Flg22) located in the N-terminus of a full length flagellin coding sequence. The elicitor activity of Flg22 polypeptide is attributed to this conserved domain within the N-terminus of the flagellin protein (Felix et al., 1999). Plants can perceive bacterial flagellin through a pattern recognition receptor (PRR) at the plant's cell surface known as flagellin sensitive receptor, which is a leucine-rich repeat receptor kinase located in the plasma membrane and available at the plant cell surface. In plants, the best-characterized PRR is FLAGELLIN SENSING 2 (FLS2), which is highly conserved in both monocot and dicot plants.

Plant defensins are also characterized as anti-microbial peptides (AMPs). Plant defensins contain several conserved cysteinyl residues that form disulfide bridges and contribute to their structural stability. Defensins are among the best characterized cysteine-rich AMPs in plants. Members of the defensin family have four disulfide bridges that fold into a globular structure. This highly conserved structure bestows highly specialized roles in protecting plants against microbial pathogenic organisms (Nawrot et al., “Plant antimicrobial peptides,” Folia Microbiology 59: 181-196, 2014).

Thionins are cystine-rich plant AMPs classified in the defensin family and typically comprise 45-48 amino acid residues, in which 6-8 of these amino acids are cysteine that form 3-4 disulfide bonds in higher plants. Thionins have been found to be present in both monocot and dicot plants and their expression can be induced by infection with various microbes (Tam et. al., “Antimicrobial peptides from plants,” Pharmaceuticals 8: 711-757, 2015). Particular amino acids of thionins such as Lys1 and Tyr13, which are highly conserved, have been found to be vital to the functional toxicity of these AMPs.

Harpin and Harpin-Like proteins are similar to the flagellins or the flagellin-associated polypeptides. Harpins comprise a group of bacterial-derived elicitors that are derived from larger precursor proteins. Harpins are critical for the elicitation of a hypersensitive response (HR) when infiltrated into the intercellular space or apoplast of plant cells (Kim et al., “Mutational analysis of Xanthomonas harpin HpaG identifies a key functional region that elicits the hypersensitive response in nonhost plants,” Journal of Bacteriology 186: 6239-6247, 2004). Application of the distant harpin-like bioactive priming polypeptide(s) to a plant provides an alternative conduit to protect a plant from disease and insect pressure. Harpins utilize a type III secretion system that enable the transport of proteins across the lipid bilayers that makeup the plant plasma cell membrane. The binding of harpins to the surface of the plasma cell membrane can trigger an innate immune response that resembles those triggered by pathogen-associated molecular patterns (PAMPs) and are known to activate PAMP-triggered immunity (Engelhardt et al., “Separable roles of the Pseudomonas syringae pv. phaseolicola accessory protein HrpZ1 in ion-conducting pore formation and activation of plant immunity,” The Plant Journal 57: 706-717, 2009). Mutational analysis of a harpin-like HpaG derived polypeptide showed that the 12 amino acid residues between Leu-39 and Leu50 of the original 133 amino acid harpin elicitor precursor protein was critical to the elicitation of a hypersensitive (HR) and subsequent innate immune responses in tobacco (Kim et al., “Mutational analysis of Xanthomonas harpin HpaG identifies a key functional region that elicits the hypersensitive response in nonhost plants,” Journal of Bacteriology 186: 6239-6247, 2004). This indicates that a specific amino acid region of harpins (similar to the other AMPs) is responsible for the elicitation responses. Harpins, such as HpaG-like can be used to enhance resistance to not only plant pathogens but also to insects (Choi et al., “Harpins, multifunctional proteins secreted by gram-negative plant pathogenic bacteria,” Molecular Plant Microbe Interactions 26: 1115-1122, 2013). Harpin has been used to induce disease resistance in plants and protect plants from colonization and feeding by insect phloem-feeding insects, such as aphids (Zhang et al., “Harpin-induced expression and transgenic overexpression of phloem protein gene At.PP2A1 in Arabidopsis repress phloem feeding of the green peach aphid Myzus persicae,” BMC Plant Biology 11: 1-11, 2011).

In some embodiments, harpin protein or polypeptides or harpin-like polypeptide can include, but is not limited to, homologs of Erwinia amylovora HrpN, which include those from species of Erwinia, Pantoea, and Pectobacterium. Examples of such homologs include those harpin proteins identified at Genbank Accession Nos. AAC31644 (Erwinia amylovora); AAQ21220, AAQ 17045, CAE25423, CAE25424, CAE25425, and CAF74881 (Erwinia pyrifoliae); CAC20124, Q47278, Q47279, and AAY17519 (Erwinia chrysanthemi); CAE25422 (Erwinia strain JP557); AAG01466 (Pantoea stewartii); AAF76342 (Pantoea agglomerans); ABZ05760, ABI15988, ABI15989, ABI15990, ABI15991, ABI15992, ABI15996, ABK80762, ABD04037, ABI15994, ABD04035, ABD04036, AAY17521, AAX38231, AB115995, AAQ73910, and CAL69276 (Pectobacterium carotovorum); YP_050198, AAS20361, and CAE45180 (Pectobacterium atrosepticum); and ABD22989 (Pectobacterium betavasculorum); each of which is hereby incorporated by reference in its entirety.

Another group of harpin protein or polypeptides or harpin-like polypeptide can include, but is not limited to, homologs of Erwinia amylovora HrpW and Pseudomonas syringae HrpW, which includes those from species of Erwinia, Pseudomonas, Xanthomonas, Acidovorax, and Pectobacterium. Examples of such homologs include those harpin proteins identified at Genbank Accession Nos. U94513, CAA74158, AAC04849, and AAF63402 (Erwinia amylovora); AAQ 17046 (Erwinia pyrifoliae); YP OO 1906489 (Erwinia tasmaniensis); YP_050207 (Pectobacterium atrosepticum); AF037983 (Pseudomonas syringae pv. tomato); AAO50075 (Pseudomonas syringae pv. phaseolicola); AAL84244 (Pseudomonas syringae pv. maculicola); AAX58537, AAX58557, AAX58525, AAX58531, AAX58527, AAX58577, AAX58491, AAX58515, AAX58517, AAX58523, AAX58583, AAX58451, AAX58561, AAX58453, AAX58541, AAX58589, AAT96311, AAX58497, AAX58579, AAX58449, AAX58485, AAX58563, AAX58581, AAX58575, AAX58569, AAX58567, AAX58505, AAX58591, AAX58503, AAX58507, AAX58509, AAX58469, AAX58441, AAX58543, AAX58495, AAX58549, AAX58593, AAX58511, AAX58519, AAT96270, AAX58447, AAX58571, AAX58465, AAX58489, AAX58533, AAX58535, AAX58461, AAT96350, AAX58473, AAX58483, AAX58475, AAX58457, AAX52461, AAX52457, AAT96222, (Pseudomonas viridiflava); ABA47299 and BAG24117 (Pseudomonas cichorii); CAH57075 (Pseudomonas avellanae); BAE80274 and BAE80242 (Acidovorax avenae); and AAM37767 (Xanthomonas axonopodis pv. citri); each of which is hereby incorporated by reference in its entirety.

Yet another group of harpin protein or polypeptides or harpin-like polypeptide can include, but is not limited to, homologs homologs of Pseudomonas syringae HrpZ, which includes those from other species of Pseudomonas. Examples of such homologs include those harpin proteins identified at Genbank Accession Nos. P35674, AAB00127, ABL01505, AAQ92359, BAD20880, BAD20876, BAD20892, BAD20884, BAD20928, BAD20936, BAD20932, BAD20924, BAD20856, BAD20864, BAD20860, BAD20848, BAD20844, BAD20836, BAD20840, BAD20824, BAD20842, BAD20820, BAD20916, BAD20872, BAC81526, 087653, BAA74798, BAD20904, AAB86735, BAD20912, BAD20908, ABL01504, BAB40655, ABO26225, ABO26228 (Pseudomonas syringae pv.); BAD20868 (Pseudomonas ficuserectae); AAX52452, AAT96159, AAX52266, AAX52396, AAT96322, AAT96281, AAX52272, AAX52306, AAX52270, AAX52402, AAX52276, AAX52318, AAX52262, and AAT96361 (Pseudomonas viridiflava); CAJ76697 (Pseudomonas avellanae); YP OOl 185537 (Pseudomonas mendocina); and ABA47309 and BAG24128 (Pseudomonas cichorii); each of which is hereby incorporated by reference in its entirety. An additional group of harpin protein or polypeptides or harpin-like polypeptide can include, but is not limited to, homologs of Xanthomonas campestris HreX (see U.S. Pat. No. 6,960,705 to Wei et al., which is hereby incorporated by reference in its entirety), which includes those from other species of Xanthomonas. Examples of such homologs include those harpin proteins identified at Genbank Accession Nos. NP_636614, YP_001904470, YP_362171 (Xanthomonas campestris); ABB72197, ABK51585, ABU48601, ABK51584, YPJ98734, and ZP_02245223 (Xanthomonas oryzae); and ABK51588 and NP_640771 (Xanthomonas axonopodis); each of which is hereby incorporated by reference in its entirety.

In some embodiments, the harpin protein or polypeptides or harpin-like polypeptide is a fragment or combination of fragments (i.e., a fusion protein) of one of the above referenced harpin proteins. In some embodiments, the harpin fragment or fusion protein includes fragments that elicit the hypersensitive response. In another embodiment, the harpin fragment or fusion protein includes fragments that do not elicit the hypersensitive response. Suitable harpin fragments include, e.g., two structural units: a stable α-helix unit with 12 or more amino acids in length; and a hydrophilic, acidic unit with 12 or more amino acids in length, which could be a beta-form, a beta-turn, or unordered forms. Fragments may also be characterized by an acidic pi value that is preferably about 5 or below. Fragments may contain any number of amino acids, e.g., between about 25 and about 60, or between about 28 to about 40 amino acids.

Examples of suitable harpin fragments or fusion protein are identified in U.S. Pat. No. 6,583,107 to Laby et al, and PCT Publication No. WO 01/098501 to Fan et al, each of which is hereby incorporated by reference in its entirety. PCT Publication No. WO 01/098501 to Fan et al. also describes methods for obtaining fragments of harpin protein or polypeptides that could be employed in the present invention.

One harpin fragment or fusion protein, now commercially available from Plant Health Care Inc., is characterized by the amino acid sequence of SEQ ID NO: 129 as follows:

MSLNTSGLGASTMQISIGGAGGNNGLLGTHMPGTSSSPGLFQSGGDNGLG
GHNANSALGQQPIDRQTIEQMAQLLAELLKSLLDSGEKLGDNFGASADSA
SGTGQQDLMTQVLNGLAKSMLDDLLTKQDGGTSFSEDDSGPAKDGNANAG
ANDPSKNDPSKSQGPQSANKTGNVDDANNQDPMQALMQLLEDLVKLLKAA
LHMQQPGGNDKGNGVGGDSGQNDDSTSGTDSTSDSSDPMQQLLKMFSEIM
QSLFGDEQDGTDSTSGSRFTRTGIGMKAGIQALNDIGTHSDSSTRSFVNK
GDRAMAKEIGQFMDQYPEVFGKPQYQKGPGQEVKTDDKSWAKALSKPDDD
GMTPASMEQFNKAKGMIKSAMAGDTGNGNLQARGAGGSSLGIDAMMAGDA
INMALGKLGAA

The harpin fragment or fusion protein of SEQ ID NO: 129 is encoded by the nucleotide sequence of SEQ ID NO: 130 as follows:

ATGAGTCTGAATACAAGTGGGCTGGGAGCGTCAACGATGCAAATTTCATC
GGCGGTGCGGGCGGAAATAACGGGTTGCTGGGTACGCATATGCCCGGGAC
CTCGTCCTCGCCGGGTCTGTTCCAGTCCGGGGGGGACAACGGGCTTGGTG
GTCATAATGCAAATTCTGCGTTGGGGCAACAACCCATCGATCGGCAAACC
AGAGCAAATGGCTCAATTATTGGCGGAACTGTTAAAGTCACTGCTAGATA
GTGGGGAAAAGCTCGGTGACAACCGGCGCGTCTGCGGACAGCGCCTCGGG
TACCGGACAGCAGGACCTGATGACTCAGGTGCTCAATGGCCTGGCCAAGT
CGATGCTCGATGATCTTCTGACCAAGCAGGATGGCGGGACCAGCTTCTCC
GAAGACGATAGTGGGCCGGCGAAGGACGGCAATGCCAACGCGGGCGCCAA
CGACCCGAGCAAGAACGACCCGAGCAAGAGCCAGGGTCCGCAGTCGGCCA
ACAAGACCGGCAACGTCGACGACGCCAACAACCAGGATCCGATGCAAGCG
CTGATGCAGCTGCTGGAAGACCTGGTGAAGCTGCTGAAGGCGGCCCTGCA
CATGCAGCAGCCCGGCGGCAATGACAAGGGCAACGGCGTGGGCGGTGATA
GTGGGCAAAACGACGATTCCACCTCCGGCACAGATTCCACCTCAGACTCC
AGCGACCCGATGCAGCAGCTGCTGAAGATGTTCAGCGAGATAATGCAAAG
CCTGTTTGGTGATGAGCAAGATGGCACCGATAGTACTAGCGGCTCGAGGT
TTACTCGTACCGGTATCGGTATGAAAGCGGGCATTCAGGCGCTGAATGAT
ATCGGTACGCACAGCGACAGCAACCCGTTCTTTCGTCAATAAAGGCGATC
GGGCGATGGCGAAGGAAATCGGTCAGTTCATGGACCAGTATCCTGAGGTG
TTTGGCAAGCCGCAGTACCAGAAAGGCCCGGGTCAGGAGGTGAAAACCGA
TGACAAATCATGGGCAAAAGCACTGAGCAAGCCAGATGACGACGGAATGA
CACCAGCCAGTATGGAGCAGTTCAACAAAGCCAAGGGCATGATCAAAAGC
GCCATGGCGGGTGATACCGGCAACGGCAACCTGCAGGCACGCGGTGCCGG
TGGTTCTTCGCTGGGTATTGATGCCATGATGGCCGGTGATGCCATTAACA
ATATGGCACTTGGCAAGCTGGGCGCGGCTTAA.

Elongation factor Tu is an abundant protein found in bacteria and acts as a pathogen-associated molecular pattern (PAMP) to initiate signaling cascades that are involved in plant disease resistance and plant innate immunity to microbial pathogenic organisms. Interestingly, some EF-Tu polypeptides are also found to exist in plants. The first 18 amino acid residues of the N-terminus of EF-Tu from Escherichia coli, termed elf18, is known to be a potent inducer of PAMP-triggered immune responses in plants (Zipfel et al., “Perception of the bacterial PAMP EF-Tu by the Receptor EFR restricts Agrobacterium-mediated transformation,” Cell 125: 749-760, 2006). Polypeptides derived from E. coli EF-Tu are perceived by the plant cell-surface localized receptor EF-Tu receptor (EFR) (Zipfel et al., 2006). EF-Tu binding and activation of EFR follow a similar mode of action compared to that of the Flg peptide-FLS2 receptor complex (Mbengue et al., “Clathrin-dependent endocytosis is required for immunity mediated by pattern recognition receptor kinases,” Proc Natl Acad Sci U.S.A. 113: 11034-9, 2016).

Phytosulfokines (PSK) belong to a group of sulfated plant polypeptides that are encoded by precursor genes that are ubiquitously present and highly conserved in higher plants (Sauter M., “Phytosulfokine peptide signaling,” Journal of Experimental Biology 66: 1-9, 2015). PSK genes are encoded by small gene families that are present in both monocots and dicots and encode a PSK polypeptide(s) that can be active as either a pentapeptide or a C-terminally truncated tetrapeptide (Lorbiecke R, Sauter M, “Comparative analysis of PSK peptide growth factor precursor homologs,” Plant Science 163: 348-357, 2002).

The phytosulfokine protein is targeted to the secretory pathway in plants by a conserved signal polypeptide (Lorbiecke R, Sauter M, “Comparative analysis of PSK peptide growth factor precursor homologs,” Plant Science 163: 348-357, 2002). Phytosulfokines (PSK) serve as sulfated growth factors with biostimulant activities and are involved in the control of the development of root and shoot apical meristems, growth regulation and reproductive processes. PSKs have also been reported to initiate cell proliferation, differentiation of quiescent tissues and are involved in the formation and stimulation and differentiation of tracheary elements (Matsubayashi et al., “The endogenous sulfated pentapeptide phytosulfokine-α stimulates tracheary element differentiation of isolated mesophyll cells of zinnia, Plant Physiology 120: 1043-1048, 1999). PSK signaling has also been reported to be involved in the regulation of root and hypocotyl elongation that occurs in Arabidopsis seedlings (Kutschmar et al., “PSK-α promotes root growth in Arabidopsis,” New Phytologist 181: 820-831, 2009).

Root hair promoting polypeptide (RHPP) is a 12 amino acid fragment derived from soybean Kunitz trypsin inhibitor (KTI) protein, which was detected from soybean meal that was subjected to degradation using an alkaline protease from Bacillus circulans HA₁₂ (Matsumiya Y. and Kubo M. “Soybean and Nutrition, Chapter 11: Soybean Peptide: Novel plant growth promoting peptide from soybean,” Agricultural and Biological Sciences, Sheny H. E. (editor), pgs. 215-230, 2011). When applied to soybean roots, RHPP was shown to accumulate in the roots and promote root growth through the stimulation of cell division and root hair differentiation in Brassica.

### Detectable Moiety

The detectable moiety can comprise any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. In some embodiments, the label is detectable without the addition of further reagents.

In some embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene, perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)ruthenium (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitrophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can comprise Rhodamine B (Rho), fluorescein isothiocyanate (FITC), 7-amino-4-methylcoumarin (Amc), green fluorescent protein (GFP), naphthofluorescein (NF), or derivatives or combinations thereof.

The detectable moiety can be attached to the cell penetrating peptide moiety at the amino group, the carboxylate group, or the side chain of any of the amino acids of the cell penetrating peptide moiety (e.g., at the amino group, the carboxylate group, or the side chain of any amino acid in the CPP).

### Linker

In various embodiments, the linker is covalently bound to an amino acid on the membrane translocation domain. The linker may be any moiety which conjugates the membrane translocation domain to the cargo moiety. In some embodiments, the linker can be an amino acid. In other embodiments, the precursor to the linker can be any appropriate molecule which is capable of forming two or more bonds with amino acids in the membrane translocation domain and cargo moiety. Thus, in various embodiments, the precursor of the linker has two or more functional groups, each of which are capable of forming a covalent bond to the membrane translocation domain and cargo moiety. For example, the linker can be covalently bound to the N-terminus, C-terminus, or side chain, or combinations thereof, of any amino acid in the membrane translocation domain. In particular embodiments, the linker forms a covalent bond between the membrane translocation domain and cargo moiety. In some embodiments, the linker can be an unstructured polypeptide sequence. In some embodiments, the when the linker is an unstructured polypeptide sequence it allows for the membrane translocation domain, linker, and cargo conjugate to be produced recombinantly.

In some embodiments, the linker is selected from the group consisting of at least one amino acid, alkylene, alkenylene, alkynylene, aryl, cycloalkyl, cycloalkenyl, cycloalkynyl, heterocyclyl, heteroaryl, ether, each of which can be optionally substituted as defined above. Non-limiting examples of linkers include polyethylene glycol, optionally conjugated to a lysine residue.

In some embodiments, the linker length can be from 0 to 1000 amino acids. In some embodiments, the linker can be designed to be Gly-Gly-Ser repeats. In some embodiments, the linker sequences can be a linker described in Adv Drug Deliv Rev. 2013 Oct. 15; 65(10): 1357-1369. For example, flexible linker sequences defined by (G)ₙ (n=1-10), or SEQ ID NO: 131 (GGGGS)ₙ (n=1-4), such as SEQ ID NO: 131 GGGGS, SEQ ID NO: 132 (GGGGS)₃; rigid linker sequences defined by SEQ ID NO: 133 A(EAAAK)ₙA (n=2-5), or (XP)ₙ (n=5-20) wherein X designating any amino acid, preferably Ala, Lys, or Glu such as SEQ ID NO: 134 A(EAAAK)₄ALEA(EAAAK)₄A, SEQ ID NO: 135 AEAAAKEAAAKA, SEQ ID NO: 136 PAPAP, (Ala-Pro)ₙ (n=5-17); cleavable linker sequences such as disulfide, protease sensitive sequences, (e.g., SEQ ID NO: 137 VSQTSKLTR↓AETVFPDV^{b}, SEQ ID NO: 138 PLG↓LWA^{c}, SEQ ID NO: 139 RVL↓AEA, SEQ ID NO: 140 EDVVCC↓SMSY, SEQ ID NO: 141 GGIEGR↓GS^{c}, SEQ ID NO: 142 TRHRQPR↓GWE, SEQ ID NO: 143 AGNRVRR↓SVG, SEQ ID NO: 144 RRRRRRR↓R↓R^{d}, or SEQ ID NO: 145 GFLGL↓^{e}, where ^{a}Protease sensitive cleavage sites are indicated with “↓”; ^{b}Factor XIa/FVIIa sensitive cleavage; ^{c}Matrix metalloprotease-1 sensitive cleavage sequences, one example provided here; ^{d}HIV PR (HIV-1 protease); NS3 protease (HCV protease); Factor Xa sensitive cleavage, respectively; ^{e}Furin sensitive cleavage; and ^{f}Cathepsin B sensitive cleavage).

In some embodiments, the linker is covalently bound to the N or C-terminus of an amino acid on CPP motif, or to a side chain of glutamine, asparagine, or lysine, or a modified side chain of glutamine or asparagine (e.g., a reduced side chain having an amino group). In particular embodiments, the linker forms a bond with the side chain of glutamine on the CPP motif. In other particular embodiments, the linker described herein has a structure of L-1 or L-2:

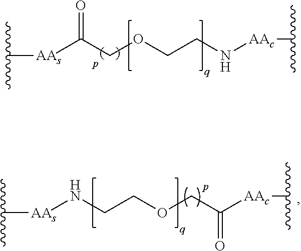

wherein
AAₛ is a side chain or terminus of an amino acid on the peptide or staple;
AA_{c} is a side chain or terminus of an amino acid of the cCPP;
p is an integer from 0 to 10; and
q is an integer from 1 to 50.

In some embodiments, the linker is capable of releasing the cargo moiety from the membrane translocation domain after the polypeptide conjugate enters the cytosol of the cell. In some embodiments, the linker contains a group, or forms a group after binding to membrane translocation domain and cargo moiety that is cleaved after cytosolic uptake of the polypeptide conjugate to thereby release the cargo moiety. Non-limiting examples of physiologically cleavable linking group include carbonate, thiocarbonate, thioether, thioester, disulfide, sulfoxide, hydrazine, protease-cleavable dipeptide linker, and the like.

For example, in embodiments, the linker is covalently bound to membrane translocation domain through a disulfide bond e.g., with the side chain of cysteine or cysteine analog located in the membrane translocation domain or cargo moiety. In some embodiments, the disulfide bond is formed between a thiol group on a precursor of the linker, and the side chain of cysteine or an amino acid analog having a thiol group on the peptide, wherein the bond to hydrogen on each of the thiol groups is replaced by a bond to a sulfur atom. Non-limiting examples of amino acid analogs having a thiol group which can be used with the polypeptide conjugates disclosed herein are discussed above.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by expressing and purifying like any other proteins. See Chen, K., & Pei, D. (2020). Engineering Cell-Permeable Proteins through Insertion of Cell-Penetrating Motifs into Surface Loops. ACS chemical biology, 15(9), 2568-2576, which is incorporated by reference herein in its entirety for its teachings of methods of preparing proteins. Other methods for preparing the disclosed compositions involve solid phase peptide synthesis wherein the amino acid α-N-terminal is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopentyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl). In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxyacetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxyacetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminal in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF.

The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either in successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Provided herein are methods of use of the compounds and/or compositions described herein. Also provided are methods of delivering a plant stimulant into a plant cell including contacting the plant cell with the peptide described herein. Also provided are methods of delivering a plant activator into a plant cell including contacting the plant cell with the peptide described herein. Also provided are methods of delivering a plant bioactive moiety into a plant cell including contacting the plant cell with the peptide described herein.

Also provided are methods of delivering a plant stimulant into a plant including contacting the plant with the peptide described herein. Also provided are methods of delivering a plant activator into a plant including contacting the plant with the peptide described herein. Also provided are methods of delivering a plant bioactive moiety into a plant including contacting the plant with the peptide described herein.

Also described are methods of treating a plant that has a disease caused by a pathogenic agent. The method can include contacting a plant with an effective amount of a compound and/or composition described herein.

In some aspects, the compounds or compositions described herein can be used to protect plants against biotic stress caused by living organisms, such as fungi, bacteria, nematodes, insects, mites, and animals; stimulate seeds during germination; to protect plants against abiotic stress caused by a physical or chemical stressor of non-living origin such as the presence of harmful chemicals including salts, restricted access to water, sunscald, freeze injury, wind injury, nutrient deficiency, or improper cultural practices, such as overwatering or planting too deep; to enhance growth, yield, health, longevity, productivity, and/or vigor of a plant; and/or provide multiple disease resistance to a plant.

Provided are also methods to protect plants against biotic stress; stimulate seeds during germination; to protect plants against abiotic stress; to enhance growth, yield, health, longevity, productivity, and/or vigor of a plant; to provide multiple disease resistance to a plant; or any combination thereof. The methods can include contacting the plant cell with the peptide described herein.

The compounds or compositions described herein provides plants' resistance to a diverse range of pathogens. In some aspects, the compounds or compositions can be used as a plant stimulant for plants that have a disease caused by a pathogenic agent. The pathogenic agent can include a fungus, virus, bacterium, mycoplasm, spiroplams or viroid. Exemplary pathogens may include fungi, such as Erisyphe polygoni, Phytophthora capsicci, Verticillium dahliae and other Verticillium spp., Powdery mildew, and Fusarium spp.; bacteria, such as Pseudomonas syringae py. tomato, and viruses, such as tobacco mosaic virus and brome mosaic virus. Other exemplary pathogens include Colletotrichum lagenarum, Pyricularia oryzae, Pseudomonas lachrymans, Xanthomonas oryzae, Xanthomonas vesicatoria, Phytophthora infestants on tomatoes, Plasmopara viticola, Pseudomonas tomato, Phytophthora parasitica var. nicotiniae, Peronospora tabacina, Cercospora nicotianae, Pseudomonas tabaci, Erysiphe graminis, Phytophora medicaginis, P. megasperma, Pyricularia oryzae, Helminthosporium leaf blight such as Helminthosporium oryzae, Cochliobolus miyabeanus, Bakanae disease such as Gibberella fujikuroi, seedling blight such as Rhizopus oryzae, sheath blight such as Rhizoctonia solani, Puccinia coronata, powdery mildew such as Erysiphe graminis, Rhynchsporium secalis, Cochliobolus sativus, Helminthosporium gramineum, Pyrenophora gramineum, Pyrenophra teres, Tilletia caries, Ustilago nuda, Leptosphaeria nodorun, Septoria nodorum, Puccinia striiformis, Typhula incamata, Pseudocercosporella herpotrichoides, Calonectria graminicola, Fusarium nivale, Puccinia graminis, Typhula ishikariensis, Puccinia recondita, Puccinia triticina, Helminthosporium gramineum, Ustilago tritici, Pythium debaryanum, Fusarium nivale, Phytophthora infestans, Peronospora tabacina, Phylophthora parasitica var, mosaic disease, Pythium debaryamum, Rhizoctonia solani, Pythium aphanidermatum, Botrytis cinerea, Botrytis cinerea, Mycosphaerella arachidicola, Rosellinia nectrix, Alternaria leaf spot, and other diseases of grains, cereals, beet, leguminous plants, pomes, drupes, fruits, citrus fruit, oil plants, cucumber plants, fiber plants, lauraceae, ornamentals, and vegetables such as oil-seed rape, sunflower, carrot, pepper, strawberry, melon, kiwi fruit, onion, leek, sweet potato, fig, ume, asparagus, persimmon, soybean, adzuki-bean, watermelon, crown daisy, spinach, lettuce, asparagus, cabbages, carrots, onions, tomatoes, potatoes, paprika, tea, wheat, barley, rye, oats, rice, sorghum, sugar beet, fodder beet, apples, pears, plums, peaches, almonds, cherries, strawberries, raspberries, blackberries, beans, lentils, peas, soybeans, rape, mustard, poppy, olives, sunflowers, coconut, castor oil plants, cocoa beans, groundnuts, cucumber, marrows, melons, cotton, flax, hemp, jute, oranges, lemons, grapefruit, mandarins, avocados, cinnamon, camphor, maize, tobacco, nuts, coffee, sugar cane, tea, vines, hops, bananas, natural rubber plants, flowers, shrubs, deciduous trees and conifers, and such the like.

The compounds or compositions described herein can be effective against a wide variety of insects. European corn borer is a major pest of corn (dent and sweet corn) but also feeds on over 200 plant species, including green beans, wax beans, lima beans, soybeans, peppers, potato, tomato, and many weed species. Additional insect larval feeding pests which damage a wide variety of vegetable crops include, without limitation, beet armyworm, cabbage looper, corn ear worm, fall armyworm, diamondback moth, cabbage root maggot, onion maggot, seed corn maggot, pickleworm (melonworm), pepper maggot, and tomato pinworm.

With regard to the use of the compounds or compositions to enhance plant growth, various forms of plant growth enhancement or promotion can be achieved. This can occur as early as when plant growth begins from seeds or later in the life of a plant. For example, plant growth according to the present invention encompasses greater yield, increased quantity of seeds produced, increased percentage of seeds germinated, increased plant size, greater biomass, more and bigger fruit, earlier fruit coloration, and earlier fruit and plant maturation. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land. In some embodiments, the compounds or compositions can be used to promote early flowering.

As used herein, “health of a plant” or “plant health” means the condition of a plant and/or its products which is determined by several aspects alone or in combination with each other, such as increased yield, plant vigor, quality, and tolerance to abiotic and/or biotic stress.

A plant suffering from fungal or insecticidal attack often produces a smaller biomass, which leads to a reduced yield as compared to a plant which has been subjected to curative or preventive treatment against the pathogenic fungus or any other relevant pest and which can grow without the damage caused by the biotic stress factor. However, applying a compound and/or composition described herein leads to enhanced plant health even in the absence of any biotic stress. The application of the compound and/or composition described herein to a plant and/or area of cultivation can also be carried out in the absence of pest pressure on the plant.

According to the present invention, “increasing yield of a plant” means that the yield of a product of the plant is increased by a measurable amount over the yield of the same product of the plant produced under the same conditions, but without application of the compound and/or composition to the plant and/or area of cultivation. In one embodiment, the term “yield” refers to fruits in the proper sense, as well as vegetables, nuts, grains, and seeds.

“Grain” and “fruit” are to be understood as any plant product which is further utilized after harvesting, e.g., fruits in the proper sense, vegetables, nuts, grains, seeds, wood (e.g., in the case of silviculture plants), flowers (e.g., in the case of gardening plants and ornamentals), etc.

Increased yield of a plant can be characterized by the following non-limiting properties: increased plant weight; increased biomass, such as higher overall fresh weight (FW) and/or higher overall dry weight (DW); increased number of flowers per plant; higher grain and/or fruit yield; more tillers or side shoots (branches); larger leaves; increased shoot growth; increased protein content; increased oil content; increased starch content; increased pigment content; increased chlorophyll content; and any combination thereof.

Chlorophyll content has a positive correlation with a plant's photosynthesis rate and, accordingly, the higher the chlorophyll content the higher the yield of a plant.

Increasing the yield of a plant may involve improving plant vigor. Plant vigor becomes manifest in several aspects, including the general visual appearance of the plant. Improved plant vigor can be characterized by, inter alia, the following: improved vitality of the plant; improved plant growth; improved plant development; improved visual appearance; improved plant stand (less plant verse/lodging); improved emergence; enhanced root growth and/or more developed root system; enhanced nodulation, in particular rhizobial nodulation; bigger leaf blade; bigger size; increased plant height; increased tiller number; increased number of side shoots; increased number of flowers per plant; increased shoot growth; increased root growth (extensive root system); enhanced photosynthetic activity; enhanced pigment content; earlier flowering; earlier fruiting; earlier and improved germination; earlier grain maturity; fewer non-productive tillers; fewer dead basal leaves; less input needed (such as fertilizers or water); greener leaves; complete maturation under shortened vegetation periods; less fertilizer needed; fewer sowing of seeds needed; easier harvesting; faster and more uniform ripening; longer shelf-life; longer panicles; delay of senescence; stronger and/or more productive tillers; better extractability of ingredients; improved quality of seeds (for being seeded in the following seasons for seed production); reduced production of ethylene and/or the inhibition of its reception by the plant; and any combination thereof.

Enhanced photosynthetic activity of a plant may be based on increased stomatal conductance and/or an increased CO₂ assimilation rate of the plant.

Increasing the yield of a plant may involve improving the quality of a plant and/or its products. Improvements in plant quality may include, without limitation, improving certain plant characteristics, such as increasing the content and/or composition of certain ingredients by a measurable or noticeable amount over the same factor of the plant produced under the same conditions, but without application of the composition of the present invention. Enhanced quality can be characterized by, inter alia, the following: increased nutrient content; increased protein content; increased content of fatty acids; increased metabolite content; increased carotenoid content; increased sugar content; increased amount of essential amino acids; improved nutrient composition; improved protein composition; improved composition of fatty acids; improved metabolite composition; improved carotenoid composition; improved sugar composition; improved amino acids composition; improved or optimal fruit color; improved leaf color; higher storage capacity; higher processability of the harvested products; or any combination thereof.

Increasing the yield of a plant may involve improving a plant's tolerance or resistance to biotic and/or abiotic stress factors. Biotic and abiotic stress, especially over longer terms, can have harmful effects on plants. Biotic stress is caused by living organisms while abiotic stress is caused, for example, by environmental extremes. In one embodiment, applying the compound and/or composition described herein to a plant pursuant to the method of the present invention enhances tolerance or resistance to biotic and/or abiotic stress factors, meaning: (1) certain negative factors caused by biotic and/or abiotic stress are diminished in a measurable or noticeable amount as compared to plants exposed to the same conditions, but without being treated with a compound and/or composition described herein and (2) the negative factors are not diminished by a direct action of the composition on the stress factors, e.g., by its fungicidal or insecticidal action which directly destroys the microorganisms or pests, but rather by a stimulation of the plants' own defensive reactions against said stress factors.

Negative factors caused by biotic stress, such as pathogens and pests, are widely known and range from dotted leaves to total destruction of the plant. Biotic stress can be caused by living organisms, such as pests (e.g., insects, arachnides, and nematodes), competing plants (e.g., weeds), microorganisms (e.g., phytopathogenic fungi and/or bacteria), and/or viruses.

Negative factors caused by abiotic stress are also well-known and can often be observed either as reduced plant vigor (as described above) or by the following symptoms: dotted leaves, “burned” leaves, reduced growth, fewer flowers, less biomass, less crop yield, reduced nutritional value of the crop, and later crop maturity, to give just a few examples. Abiotic stress can be caused by, inter alia: extremes in temperature such as heat or cold (heat stress/cold stress), strong variations in temperature, temperatures unusual for the specific season, drought (drought stress), extreme wetness, high salinity (salt stress), radiation (e.g., by increased UV radiation due to the decreasing ozone layer), increased ozone levels (ozone stress), organic pollution (e.g., by phytotoxic amounts of pesticides), inorganic pollution (e.g., by heavy metal contaminants), and any combination thereof.

Biotic and/or abiotic stress factors decrease the quantity and the quality of the stressed plants, their crops, and fruits. As far as quality is concerned, reproductive development can be affected with consequences on the crops which are important for fruits or seeds. Synthesis, accumulation, and storage of proteins are mostly affected by temperature; growth is slowed by almost all types of stress; polysaccharide synthesis, both structural and storage, is reduced or modified. These effects result in a decrease in biomass (yield) and in changes in the nutritional value of the plant product.

The above identified indicators for the health condition of a plant may be interdependent and may result from each other. For example, an increased resistance to biotic and/or abiotic stress may lead to a better plant vigor, e.g., to better and bigger crops, and thus to an increased yield. Inversely, a more developed root system may result in an increased resistance to biotic and/or abiotic stress.

Applying the compound and/or composition described herein to a plant and/or area of cultivation can have a synergistic effect on the plant to: increase the health of the plant, increase the yield of the plant, increase the biomass of the plant, increase the oil content of the plant, increase the vigor of the plant, increase the stand of the plant, increase the emergence of the plant, increase the root growth of the plant, increase the photosynthetic activity of the plant, improve the quality of the plant, improve the nutrient composition of the plant, improve the protein composition of the plant, improve the carotenoid composition of the plant, increase the tolerance of the plant to biotic stress, increase the tolerance of the plant to fungi, increase the tolerance of the plant to nematodes, increase the tolerance of the plant to bacteria, increase the tolerance of the plant to abiotic stress, increase the tolerance of the plant to drought stress, increase the tolerance of the plant to cold stress, increase the tolerance of the plant to heat stress, increase the tolerance of the plant to salt stress, increase the tolerance of the plant to ozone stress, and/or any combination thereof.

One of the most important factors for increased resistance against biotic and abiotic stress is the stimulation of the plant's natural defense reactions, which occurs by application of compound and/or composition described herein according to the method described herein.

The plant, including its roots, flowers, leaves, or stems, can be contacted with the disclosed compounds or compositions in any known technique for applying plant formulations.

Exemplary application techniques include, but are not limited to, spraying, atomizing, dusting, spreading, sprinkling, dripping, dipping, drenching, injecting, hydrophonics, or direct application into water (in-water). In some embodiments, suitable compositions can include those for HV, LV, and ULV spraying and for ULV cool and warm fogging formulations. The method of application can vary depending on the intended purpose. The compositions can be applied on the plants in a field or in a greenhouse. In some aspects, the compositions can be applied to a portion of the plant, for example, to the tubers before planting. In some embodiments, the compounds or compositions can be applied on the plants surface or plant plasma membrane as a foliar spray.

The composition can be contacted with any part of the plant, for example, the root or the leaves of the plant. In some embodiments, the composition can be contacted to the roots by spraying the soil, mechanical incorporation, mixed with fertilizer, soil improvement, pre-mix or such the like.

In some embodiments, the composition can be contacted to a plant seed. Seed treatments containing the compounds or compositions can be applied using any commercially available seed treatment machinery or can also be applied using any acceptable non-commercial method(s) such as the use of syringes or any other seed treatment device. General seed treatments coating procedures using compounds or compositions can be performed using a Wintersteiger HEGE 11 (Wintersteiger AG, Austria, Germany) and applied to the seed of major crops, namely corn, soybean, wheat, rice and various vegetables.

The seeds can be coated using a variety of methods including, but not limited to, pouring or pumping, drizzling or spraying an aqueous solution containing the compounds or compositions on or over a seed, spraying or applying onto a layer of seeds either with the use or without the use of a conveyor system. Suitable mixing devices include tumblers, mixing basins or drums, or other fluid applicating devices that include basins or drums used to contain the seed while coating.

The compositions described herein can be contacted intermittently to the plant. In some aspects, the plant can be contacted with the composition two times of greater. For example, the plant can be contacted with the composition 3, 4, 5, 6, 7, 8, 9, or 10 times. In some embodiments, the plant can be contacted with the composition from 2 to about 5 times. In some embodiments, the plant can be contacted with the composition once. In some aspects, the plant can be contacted with the composition once every 5 to 21 days. For example, the plant can be contacted with the composition once every 5, 6, 7, 8, 9, 10, 11, 12, 13, 14, 15, 16, 17, 18, 19, 20, or 21 days. In some embodiments, the plant can be contacted with the composition once per week. In some aspects, the plant can be contacted with the composition 1 to 5 times per 5 to 21 days. For example, the plant can be contacted about 1 to about 5 times per week.

In some aspects the compositions described herein can be applied before the stressing factor(s) appears.

The term “plant” as used herein includes whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. A “plant cell” as used herein refers to any plant cell and can comprise a cell at the plant surface or internal to the plant plasma membrane, for example, an epidermal cell, a trichome cell, a xylem cell, a phloem cell, a sieve tube element, or a companion cell.

The class of plants that can be used in the methods described herein include the class of higher and lower plants, including angiosperms (monocotyledonous and dicotyledonous plants), gymnosperms, ferns, horsetails, psilophytes, lycophytes, bryophytes, and multicellular algae. For example, plants for use in the methods described herein include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dio-scorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits, onions (including garlic, shallots, leeks, and chives); fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat preferred. In some embodiments, plants for use in the methods described herein include any crop plant, for example, forage crop, oilseed crop, grain crop, fruit crop, vegetable crop, fiber crop, spice crop, nut crop, turf crop, sugar crop, beverage crop, and forest crop.

Plants may be categorized into agricultural, silvicultural, ornamental, and horticultural plants, based on their human use and/or consumption. In addition, “plants” include natural or wildtype plants, and plants that have been genetically modified.

“Agricultural” plants are plants of which a part or all is harvested or cultivated on a commercial scale or which serve as an important source of feed, food, fibers (e.g., cotton and linen), combustibles (e.g., wood, bioethanol, biodiesel, and biomass) or other chemical compounds. Agricultural plants also include vegetables. Thus, agricultural plants include cereals (e.g., wheat, rye, barley, triticale, oats, sorghum, and rice); beet (e.g., sugar beet or fodder beet); leguminous plants (e.g., beans, lentils, peas, alfalfa, and soybean); oil plants (e.g., rape, oil-seed rape, canola, juncea (Brassica juncea), linseed, mustard, olive, sunflower, cocoa bean, castor oil plants, oil palms, ground nuts, and soybean); cucurbits (e.g., squash, cucumber, and melon); fiber plants (e.g., cotton, flax, hemp, and jute); vegetables (e.g., cucumbers, spinach, lettuce, asparagus, cabbages, carrots, radish, turnip, celery, chicory, endive, brussel sprouts, parsnip, cauliflower, broccoli, garlic, eggplant, pepper, pumpkin, onions, tomatoes, potatoes, sweet potatoes, cucurbits, and paprika); lauraceous plants (e.g., avocados, cinnamon, and camphor); energy and raw material plants (e.g., corn, soybean, rape, canola, sugar cane, and oil palm); tobacco; nuts (including peanuts); coffee; tea; vines (e.g., table grapes and juice grape vines); hop; stone fruit; apple; blueberry; strawberry; pear; citrus; raspberry; pineapple; sugarcane; turf, natural rubber plants, and marijuana.

“Horticultural plants” are plants commonly used in horticulture and include, without limitation, ornamentals, vegetables, and fruits. “Ornamental” plants are plants which are commonly used in gardening, e.g., in parks, gardens, and on balconies and patios. Non-limiting examples of ornamentals include turf, geranium, pelargonia, petunia, begonia, and fuchsia. Non-limiting examples of vegetables are as described above. Non-limiting examples of fruits include apples, pears, cherries, strawberry, citrus, peaches, apricots, and blueberries.

“Silvicultural” plants are understood to be trees, more specifically, trees used in reforestation or industrial plantations. Industrial plantations generally serve the purpose of commercial production of forest products such as wood, pulp, paper, rubber tree, Christmas trees, or young trees for gardening purposes. Non-limiting examples of silvicultural plants are conifers (e.g., pines), in particular Pinus species fir and spruce; eucalyptus; tropical trees (e.g., teak, rubber tree, oil palm); willow (Salix), in particular Salix species; poplar (cottonwood), in particular Populus species; beech, in particular Fagus species; birch; oil palm; cherry, walnut, and oak.

As noted above, the term “plant” also includes plants modified from their wildtype form. Such modifications may occur through breeding, mutagenesis, or genetic engineering (including transgenic and non-transgenic plants). Plants modified by genetic engineering include plants having genetic material that has been modified by the use of recombinant DNA techniques. Such modifications typically include modifications that cannot readily be obtained by cross breeding under natural circumstances, mutations, or natural recombination. Typically, one or more genes have been integrated into the genetic material of a genetically modified plant in order to improve certain properties of the plant. Examples of genetically modified plants, include but are not limited to, crops which tolerate the action of herbicides, fungicides, or insecticides owing to breeding, including genetic engineering methods, or plants which have modified characteristics in comparison with existing plants, which can be generated by, e.g., traditional breeding methods and/or the generation of mutants, or by recombinant procedures. Examples of genetically modified plants also include those that, through the use of recombinant DNA techniques, are able to synthesize one or more proteins to increase the resistance or tolerance of those plants to bacterial, viral, or fungal pathogens; to increase the productivity (e.g., biomass production, grain yield, starch content, oil content, and/or protein content), tolerance to drought, salinity, or other growth-limiting environmental factors or tolerance to pests and fungal, bacterial, or viral pathogens of those plants, and/or that contain a modified amount of substances of content or new substances of content, specifically to improve raw material production and/or to improve human or animal nutrition, e.g., potatoes that produce increased amounts of amylopectin (e.g., oil crops that produce health-promoting long-chain omega-3 fatty acids or unsaturated omega-9 fatty acids (e.g., Nexera® rape, DOW Agro Sciences, Canada), Amflora® potato, BASF SE, Germany).

The methods for producing such genetically modified plants are generally known to the person of ordinary skill in the art and are described, e.g., in the above-noted references.

### Compositions, Formulations and Methods of Administration

Also disclosed herein are compositions comprising the compounds described herein.

Agricultural formulations of active substances are well known. Non-limiting examples include a solid, a semi-solid, a liquid, a solution, a suspension, an emulsion, a gel, an oil dispersion, capsule (such as the active ingredient encapsulated in a microcapsule), dusts, powders, pastes, granules, or the like. The particular formulation chosen may vary depending on the particular intended mode of administration. In each case, it is typically an advantage to ensure a fine and even distribution of the active ingredient(s) in a liquid or solid carrier. The compositions described herein can be in any suitable form based on its intended use. In some aspects, the compositions can be in the form of an aqueous solution. In some aspects, the compositions can be a solution comprising an organic solvent, such as an alcohol. In some aspects, the compositions can be a solution comprising a mixture of organic and inorganic solvents.

Preferably, the composition described herein is formulated in a manner suitable for large or small scale agricultural and horticultural applications.

The selected dosage level of the composition will depend upon a variety of factors including for example, the route of administration, the time of administration, the duration of the treatment, other drugs and/or materials used in combination with the particular compound employed, the condition and general health of the plant being treated, and like factors well-known in the agricultural arts. However, the compositions described herein provides plant stimulation even at low doses. A person having ordinary skill in the art can readily determine and prescribe the effective amount of the composition required.

Formulation methods are taught, e.g., in U.S. Pat. No. 3,060,084 to Littler and European Patent No. 0707445 to BASF AG (for liquid concentrates); Browning, “Agglomeration,” Chemical Engineering pp. 147-48 (1967); Perry's Chemical Engineer's Handbook, 4th Ed., McGraw-Hill, New York, 1963; PCT Publication No. WO 91/13546 to E.I. Du Pont De Nemours and Co.; U.S. Pat. No. 4,172,714 to Albert; U.S. Pat. No. 4,144,050 to Frensch et al.; U.S. Pat. No. 3,920,442 to Albert; U.S. Pat. No. 5,180,587 to Moore; U.S. Pat. No. 5,232,701 to Ogawa et al.; U.S. Pat. No. 5,208,030 to Hoy et al., Great Britain Patent No. 2,095,558; U.S. Pat. No. 3,299,566 to Macmullen; Klingman, Weed Control as a Science, J. Wiley & Sons, New York, 1961; Hance et al., Weed Control Handbook, 8th Ed., Blackwell Scientific, Oxford, 1989; and Mollet and Grubemann, Formulation Technology, Wiley VCH Verlag, Weinheim, 2001, each of which is hereby incorporated by reference in its entirety.

The compositions can include, as noted above, an agriculturally effective amount of the compound described herein in combination with an agriculturally acceptable carrier and, in addition, can include other auxiliaries. The compositions can be formulated (either together or separately) in a manner common for agrochemical formulations. For example, the composition(s) may include auxiliaries which are customary in agrochemical formulations. The particular auxiliaries used may depend on the particular application form and active substance, respectively. Non-limiting examples of suitable auxiliaries include carriers, adjuvants, diluents, thickeners, buffers, preservatives, surfactants, wetting agent, a coating agent, a monosaccharide, a polysaccharide, an abrading agent, a pesticide, an insecticide, an herbicide, a nematicide, a bacteriocide, a fungicide, a miticide, a fertilizer, a biostimulant, a colorant, a humectant, antifreeze agents, antifoam agents, compatibilizing agents, sequestering agents, neutralizing agents and buffers, corrosion inhibitors, an osmoprotectant, odorants, an antibiotic, spreading agents, dispersing agents, freeze point depressants, antimicrobial, crop oil, safeners, adhesives, protective colloids, emulsifiers, tackifiers, an amino acid, a biological control agent, or a combination thereof.

In some embodiments, the compositions described herein can include a pesticide comprising an insecticide, a herbicide, a fungicide, a bacteriocide, a nematicide, a miticide, or any combination thereof. The pesticide can be applied to the plant simultaneously or sequentially. In some embodiments, pesticide is applied to the plant after the compound and/or composition described herein is applied.

In some aspects, concentrates, suitable for dilution, of the compositions can be prepared with the compositions, in addition to water, a wetting agent, a tackifier, a dispersant, or an emulsifier.

The agriculturally acceptable carrier can include an organic or an inorganic carrier. Exemplary carriers include, but are not limited to, water, organic solvents, inorganic solvents, petroleum fractions or hydrocarbons such as mineral oil, aromatic solvents, paraffinic oils, vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil, esters of the above vegetable oils, esters of monoalcohols or dihydric, trihydric, or other lower polyalcohols (4-6 hydroxy containing), such as 2-ethyl hexyl stearate, n-butyl oleate, isopropyl myristate, propylene glycol dioleate, di-octyl succinate, di-butyl adipate, di-octyl phthalate, esters of mono, di and polycarboxylic acids, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerine, N-methyl-2-pyrrolidinone, N,N-dimethyl alkylamides, dimethyl sulfoxide, liquid fertilizers, and mixtures thereof. Other exemplary carriers include silicas, silica gels, silicates, talc, kaolin, limestone, lime, chalk, bole, loess, clay, dolomite, diatomaceous earth, calcium sulfate, magnesium sulfate, magnesium oxide, ground synthetic materials, pyrophyllite clay, attapulgus clay, kieselguhr, calcium carbonate, bentonite clay, Fuller's earth, cottonseed hulls, wheat flour, soybean flour, pumice, wood flour, walnut shell flour, lignin, ammonium sulfate, ammonium phosphate, ammonium nitrate, ureas, cereal meal, tree bark meal, wood meal and nutshell meal, cellulose powders, and mixtures thereof. The agriculturally acceptable carrier can be present in an amount of 99.9% by weight or less, 99% by weight or less, 98% by weight or less, 97% by weight or less, 95% by weight or less, 90% by weight or less, 85% by weight or less, 80% by weight or less, 75% by weight or less, 70% by weight or less, 65% by weight or less, 60% by weight or less, 55% by weight or less, 50% by weight or less, 45% by weight or less, or 40% by weight or less, based on the weight of the composition.

When the composition includes an amino acid, the amino acid can be provided separately from the amino acids that comprise the polypeptide. For example, an isolated amino acid can be used. Suitable amino acids include any natural or unnatural amino acids. For example, the composition can comprise cysteine.

Unless otherwise specified, each agriculturally acceptable auxiliary can be present from 0.1 to 60 wt. %, from 0.5 to 50 wt. %, or from 10 to 30 wt. % of the total weight of the composition.

When the composition includes a preservative, the preservative can comprise those based on dichlorophene and benzylalcohol hemi formal (PROXEL from ICI or ACTICIDE RS from Thor Chemie and KATHON MK from Dow Chemical) and isothiazolinone derivatives such as alkylisothiazolinones and benzisothiazolinones (ACTICIDE MBS from Thor Chemie). As further examples, suitable preservatives include MIT (2-methyl-4-isothiazolin-3-one), BIT (1,2-benzisothiazolin-3-one, which can be obtained from Avecia, Inc. as PROXEL GXL as a solution in sodium hydroxide and dipropylene glycol), 5-chloro-2-(4-chlorobenzyl)-3(2H)-isothiazolone, 5-chloro-2-methyl-2H-isothiazol-3-one, 5-chloro-2-methyl-2H-isothiazol-3-one, 5-chloro-2-methyl-2H-isothiazol-3-one-hydrochloride, 4,5-dichloro-2-cyclohexyl-4-isothiazolin-3-one, 4,5-dichloro-2-octyl-2H-isothiazol-3-one, 2-methyl-2H-isothiazol-3-one, 2-methyl-2H-isothiazol-3-one-calcium chloride complex, 2-octyl-2H-isothiazol-3-one, benzyl alcohol hemiformal, or any combination thereof.

When the composition includes a buffering agent, the buffering agent can comprise potassium, phosphoric acid, a phosphate salt, citric acid, a citrate salt, a sulfate salt, MOPS, or HEPES. The buffering agent can stabilize the polypeptide in the composition.

When the composition includes a wetting agent, the wetting agent can comprise organosilicones, polyoxyethoxylates, polysorbates, polyethyleneglycol and derivatives thereof, ethoxylates, crop oils, and polysaccharides.

When the composition includes a surfactant, the surfactant can comprise a heavy petroleum oil, a heavy petroleum distillate, a polyol fatty acid ester, a polyethoxylated fatty acid ester, an aryl alkyl polyoxyethylene glycol, a polyoxyethylenepolyoxypropylene monobutyl ether, an alkyl amine acetate, an alkyl aryl sulfonate, a polyhydric alcohol, an alkyl phosphate, an alcohol ethoxylate, an alkylphenol ethoxylate, an alkyphenol ethoxylate, an alkoxylated polyol, an alky polyethoxy ether, an alkylpolyoxethylene glycerol, ethoxylated and soybean oil derivatives, an organosilicone-based surfactant or any combination thereof. Surfactants can be included in a range of compositions including those for foliar use.

When the composition includes a coating agent, the coating agent can comprise a tackifier, polymers, filling agents, or bulking agents.

The tackifier can include, but is not limited to, carboxymethylcellulose and natural and synthetic polymers in the form of powders, granules, or latexes, such as gum Arabic, chitin, polyvinyl alcohol and polyvinyl acetate, as well as natural phospholipids, such as cephalins and lecithins, and synthetic phospholipids. Tackifiers include those composed preferably of an adhesive polymer that can be natural or synthetic without phytotoxic effect on the seed to be coated. Additional tackifiers that can be included, either alone or in combination, include, for example, polyesters, polyether esters, polyanhydrides, polyester urethanes, polyester amides; polyvinyl acetates; polyvinyl acetate copolymers; polyvinyl alcohols and tylose; polyvinyl alcohol copolymers; polyvinylpyrrolidones; polysaccharides, including starches, modified starches and starch derivatives, dextrins, maltodextrins, alginates, chitosans and celluloses, cellulose esters, cellulose ethers and cellulose ether esters including ethylcelluloses, methylcelluloses, hydroxymethylcelluloses, hydroxypropylcelluloses and carboxymethylcellulose; fats; oils; proteins, including casein, gelatin and zeins; gum arabics; shellacs; vinylidene chloride and vinylidene chloride copolymers; lignosulfonates, in particular calcium lignosulfonates; polyacrylates, polymethacrylates and acrylic copolymers; polyvinylacrylates; polyethylene oxide; polybutenes, polyisobutenes, polystyrene, polybutadiene, polyethyleneamines, polyethylenamides; acrylamide polymers and copolymers; polyhydroxyethyl acrylate, methylacrylamide monomers; and polychloroprene, or any combination thereof. Tackifiers can be used in a range of compositions including those for seed treatment.

When the composition includes an abrading agent, the abrading agent can comprise talc, graphite, or a combination of both.

A humectant is a hygroscopic substance that assists with the retention of moisture. When the composition includes a humectant, the humectant can comprise: glycerol, glycerin, a glycerol derivative (e.g. glycerol monosterate, glycerol triacetate, triacetin, propylene glycol, hexylene glycol, or butylene glycol), triethylene glycol, tripolypropylene glycol, glyceryl triacetate, sucrose, tagatose, a sugar alcohol or a sugar polyol (e.g glycerol, sorbitol, xylitol, mannitol, or manitol), a polymeric polyol (e.g. polydextrose, a collagen, an aloe or an aloe vera gel), or an alpha hydroxy acid (e.g. lactic acid, honey, molasses, quillaia, sodium hexametaphosphate, lithium chloride or urea). Synthetic humectants can also comprise: butylene glycol, and tremella extract.

When the compound described herein are formulated or applied in combination with commercially available fungicides, the compositions can provide an extra layer of protection for enhancing disease prevention or spread in a plant.

A variety of colorants may be employed, including organic chromophores classified as nitroso, nitro, azo, including monoazo, bisazo, and polyazo, diphenylmethane, triarylmethane, xanthene, methane, acridine, thiazole, thiazine, indamine, indophenol, azine, oxazine, anthraquinone, phthalocyanine, or any combination thereof.

Biological control agents are broadly defined as microorganisms that can be used instead of synthetic pesticides or fertilizers. When the composition includes a biological control agent, the biological control agent can comprise Bacillus thuringiensis, Bacillus megaterium, Bacillus mycoides isolate J, Bacillus methylotrophicus, Bacillus vallismortis, Chromobacterium subtsugae, Delftia acidovorans, Streptomyces lydicus, Streptomyces colombiensis, Streptomyces galbus K61, Penicillium bilaii, a lipopeptide-producing Bacillus subtilis strain, a lipopeptide-producing Bacillus amyloliquefaciens strain, a Bacillus firmus strain or a Bacillus pumilus strain.

When the composition includes a fertilizer, the fertilizer can include ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, calcium nitrate, dolomitic limestone, hydrated lime, calcium carbonate, diammonium phosphate, monoammonium phosphate, magnesium nitrate, magnesium sulfate, potassium nitrate, potassium chloride, potassium magnesium sulfate, potassium sulfate, sodium nitrates, magnesian limestone, magnesia, urea, urea-formaldehydes, urea ammonium nitrate, sulfur-coated urea, polymer-coated urea, isobutylidene diurea, K₂SO4-Mg₂SO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion, humic acid, or any combination thereof.

The fertilizer can comprise a liquid fertilizer or a dry fertilizer. The composition can include a micronutrient fertilizer material, the micronutrient fertilizer material comprising boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate, or any combination thereof.

When the composition includes a biostimulant, the biostimulant can comprise a seaweed extract, an elicitor, a polysaccharide, a monosaccharide, a protein extract, a soybean extract, a humic acid, a plant hormone, a plant growth regulator, or any combination thereof.

Examples of thickeners (i.e., compounds that impart a modified flowability to formulations (i.e., high viscosity under static conditions and low viscosity during agitation) are polysaccharides and organic and inorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

Examples of suitable anti-freezing agents are ethylene glycol, propylene glycol, urea, and glycerin.

Examples of anti-foaming agents are silicone emulsions (e.g., Silikon® SRE, Wacker, Germany and Rhodorsil®, Rhodia, France), long chain alcohols, fatty acids, salts of fatty acids, fluoroorganic compounds, and mixtures thereof.

The fungicide can comprise aldimorph, ampropylfos, ampropylfos potassium, andoprim, anilazine, azaconazole, azoxystrobin, benalaxyl, benodanil, benomyl, benzamacril, benzamacryl-isobutyl, benzovindiflupyr, bialaphos, binapacryl, biphenyl, bitertanol, blasticidin-S, boscalid, bromuconazole, bupirimate, buthiobate, calcium polysulphide, capsimycin, captafol, captan, carbendazim, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, dichlofluanid, diclomezine, dicloran, diethofencarb, dimethirimol, dimethomorph, dimoxystrobin, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, flumetover, fluoromide, fluoxastrobin fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metconazole, metalzxyl, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, prothiocinazole, pyrazophos, pyrifenox, pyrimethanil, pyroquilon, pyroxyfur, quinconazole, quintozene (PCNB), a strobilurin, sulphur and sulphur preparations, tebuconazole, tecloftalam, tecnazene, tetcyclasis, tetraconazole, thiabendazole, thicyofen, thifluzamide, thiophanate-methyl, tioxymid, tolclofos-methyl, tolylfluanid, triadimefon, triadimenol, triazbutil, a triazole, triazoxide, trichlamide, tricyclazole, triclopyr, tridemorph, trifloxystrobin, triflumizole, triforine, uniconazole, validamycin A, vinclozolin, viniconazole, zarilamide, zineb, ziram and also Dagger G, OK-8705, OK-8801, a-(1,1-dimethylethyl)-(3-(2-phenoxyethyl)-1H-1,2,4-triazole-1-ethanol, a-(2,4-dichlorophenyl)-[3-fluoro-3-propyl-1H-1,2,4-triazole-1-ethanol, a-(2,4-dichlorophenyl)-[3-methoxy-a-methyl-1H-1,2,4-triazole-1-ethanol, a-(5-methyl-1,3-dioxan-5-yl)-[3-[[4-(trifluoromethyl)-phenyl]-methylene]-1H-1,2,4-triazole-1-ethanol, (5RS,6RS)-6-hydroxy-2,2,7,7-tetramethyl-5-(1H-1,2,4-triazol-1-yl)-3-octanone, (E)-a-(methoxyimino)-N-methyl-2-phenoxy-phenylacetamide, 1-isopropyl{2-methyl-1-[[[1-(4-methylphenyl)-ethyl]-amino]-carbonyl]-propyl}carbamate, 1-(2,4-dichlorophenyl)-2-(1H-1,2,4-triazol-1-yl)-ethanone-O-(phenyl methyl)-oxime, 1-(2-methyl-1-naphthalenyl)-1H-pyrrole-2,5-dione, 1-(3,5-dichlorophenyl)-3-(2-propenyl)-2,5-pyrrolidindione, 1-[(diiodomethyl)-sulphonyl]-4-methyl-benzene, 1-[[2-(2,4-dichlorophenyl)-1, 3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluoromethoxy-4′-trifluoro-methyl-1, 3-thiazole-carboxanilide, 2,2-dichloro-N-[1-(4-chlorophenyl)-ethyl]-1-ethyl-3-methyl-cyclopropanecarboxamide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)-sulphonyl]-5-(trichloromethyl)-1,3,4-thiadiazole, 2-[[6-deoxy-4-O-(4-O-methyl-(3-D-glycopyranosyl)-a-D-glucopyranos yl]-amino]-4-methoxy-1H-pyrrolo[2,3-d]pyrimidine-5-carbonitrile, 2-aminobutane, 2-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acetamide, 2-phenylphenol (OPP), 3,4-dichloro-1-[4-(difluoromethoxy)-phenyl]-pyrrole-2,5-dione, 3,5-dichloro-N-[cyano[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetrazolo[1,5-a]quinazolin-5(4H)-one, 8-(1,1-dimethylethyl)-N-ethyl-N-propyl-1,4-dioxaspiro[4, 5]decane-2-methanamine, 8-hydroxyquinoline sulphate, 9H-xanthene-2-[(phenylamino)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1-dimethylpropyl)-phenyl-2-methylpropyl]-2,6-dimethyl-morpholine hydrochloride, ethyl[(4-chlorophenyl)-azo]-cyanoacetate, potassium bicarbonate, methanetetrathiol-sodium salt, methyl 1-(2,3-dihydro-2,2-dimethyl-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro-4-hydroxyphenyl)-1-methyl-cyclohexanecarboxamide, N-(2,6-dimethyl phenyl)-2-methoxy-N-(tetra hydro-2-oxo-3-furanyl)-acetamide, N-(2,6-dimethyl phenyl)-2-methoxy-N-(tetrahydro-2-oxo-3-thienyl)-acetamide, N-(2-chloro-4-nitrophenyl)-4-methyl-3-nitro-benzenesulphonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy)-3-pyridinyl)-cyclopropanecarboxamide, N-[2,2,2-trichloro-1-[(chloroacetyl)-amino]-ethyl]-benzamide, N-[3-chloro-4,5-bis(2-propinyloxy)-phenyl]-N′-methoxy-methanimidamide, N-formyl-N-hydroxy-DL-alanine-sodium salt, O,O-diethyl [2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, and spiro[2H]-1-benzopyrane-2,1′(3′H)-isobenzofuran]-3′-one, N-trichloromethyl)thio-4-cyclohexane-1,2-dicarboximide, tetramethylthioperoxydicarbonic diamide, methyl N-(2,6-dimethylphenyl)-N-(methoxyacetyl)-DL-alaninate, 4-(2,2-difluoro-1,3-benzodioxol-4-yl)-1-H-pyrrol-3-carbonitril, or any combination thereof.

The strobilurin fungicide can comprise a Strobilurin A, a Strobilurin B, a Strobilurin C, a Strobilurin D, a Strobilurin E, a Strobilurin F, a Strobilurin G, a Strobilurin H, an Azoxystrobin, a Trifloxystrobin, a Kresoxim methyl, a Fluoxastrobin, Picoxystrobin, or any combination thereof.

The strobilurin fungicide can comprise a non-naturally occurring strobilurin fungicide such as an Azoxystrobin, a Trifloxystrobin, a Kresoxim methyl, a Fluoxastrobin, or any combination thereof. For example, the strobilurin fungicide can comprise a Trifloxystrobin, Fluoxastrobin or Picoxystrobin. Strobilurin fungicides are used to control a range of fungal diseases, including water molds, downy mildews, powdery mildews, leaf spotting and blighting fungi, fruit rotters, and rusts. They are useful for treating a variety of crops, including cereals, field crops, fruits, tree nuts, vegetables, turfgrasses, and ornamentals.

The triazole fungicide can comprise prothioconazole, imidazole, imidazil, prochloraz, propiconazole, triflumizole, diniconazole, flusilazole, penconazole, hexaconazole, cyproconazole, myclobutanil, tebuconazole, difenoconazole, tetraconazole, fenbuconazole, epoxiconazole, metconazole, fluquinconazole, triticonazole, or any combination thereof.

In addition, the fungicide can comprise azoxystrobin, carboxin, difenoconazole, fludioxonil, fluxapyroxad, ipconazole, mefenoxam, pyraclostrobin, silthiofam, sedaxane, thiram, triticonazole or any combination thereof.

The herbicide can comprise 2,4-D, 2,4-DB, acetochlor, acifluorfen, alachlor, ametryn, atrazine, aminopyralid, benefin, bensulfuron, bensulfuron methyl bensulide, bentazon, bispyribac sodium, bromacil, bromoxynil, butylate, carfentrazone, chlorimuron, 2-chlorophenoxy acetic acid, chlorsulfuron, chlorimuron ethyl, clethodim, clomazone, clopyralid, cloransulam, CMPP-P-DMA, cycloate, DCPA, desmedipham, dicamba, dichlobenil, diclofop, 2,4-dichlorophenol, dichlorophenoxyacetic acid, dichlorprop, dichlorprop-P, diclosulam, diflufenzopyr, dimethenamid, dimethyl amine salt of 2,4-dichlorophenoxyacetic acid, diquat, diuron, DSMA, endothall, EPTC, ethalfluralin, ethofumesate, fenoxaprop, fluazifop-P, flucarbazone, flufenacet, flumetsulam, flumiclorac, flumioxazin, fluometuron, fluroxypyr, fluroxypyr 1-methyleptylester, fomesafen, fomesafen sodium salt, foramsulfuron, glufosinate, glufosinate-ammonium, glyphosate, halosulfuron, halosulfuron-methyl, hexazinone, 2-hydroxyphenoxy acetic acid, 4-hydroxyphenoxy acetic acid, imazamethabenz, imazamox, imazapic, imazaquin, imazethapyr, isoxaben, isoxaflutole, lactofen, linuron, mazapyr, MCPA, MCPB, mecoprop, mecoprop-P, mesotrione, metolachlor-s, metribuzin, metsulfuron, metsulfuron-methyl, molinate, MSMA, napropamide, naptalam, nicosulfuron, norflurazon, oryzalin, oxadiazon, oxyfluorfen, paraquat, pelargonic acid, pendimethalin, phenmedipham, picloram, prim isulfuron, prodiamine, prometryn, pronamide, propanil, prosulfuron, pyrazon, pyrithiobac, pyroxasulfone, quinclorac, quizalofop, rimsulfuron, sethoxydim, siduron, simazine, sulfentrazone, sulfometuron, sulfosulfuron, tebuthiuron, terbacil, thiazopyr, thifensulfuron, thifensulfuron-methyl, thiobencarb, tralkoxydim, triallate, triasulfuron, tribenuron, tribernuron-methyl, triclopyr, trifluralin, triflusulfuron, or any combination thereof.

When the composition includes a nematicide, the nematicide can comprise Bacillus firmus, fluopyram, antibiotic nematicides such as abamectin; carbamate nematicides such as acetoprole, Bacillus chitonosporus, chloropicrin, benclothiaz, benomyl, Burkholderia cepacia, carbofuran, carbosulfan, and cleothocard; dazomet, DBCP, DCIP, alanycarb, aldicarb, aldoxycarb, oxamyl, diamidafos, fenamiphos, fosthietan, phosphamidon, cadusafos, chlorpyrifos, diclofenthion, dimethoate, ethoprophos, fensulfothion, fostiazate, harpins, heterophos, imicyafos, isamidofos, isazofos, methomyl, mecarphon, Myrothecium verrucaria, Paecilomyces lilacinus, Pasteuria nishizawae (including spores thereof), phorate, phosphocarb, terbufos, thionazin, triazophos, tioxazafen, dazomet, 1,2-dichloropropane, 1,3-dichloropropene, furfural, iodomethane, metam, methyl bromide, methyl isothiocyanate, xylenol, or any combination thereof. For example, the nematicide can comprise Bacillus firmus strain i-2580. Pasteuria nishizawae (including spores thereof), or fluopyram.

When the composition includes a bacteriocide, the bacteriocide can comprise streptomycin, penicillins, tetracyclines, oxytetracycline, kasugamycin, ampicillin, oxolinic acid, chlorotetracycline, copper oxide, or any combination thereof. For example, the bacteriocide can comprise oxytetracycline.

When the composition includes an insecticide, the insecticide can comprise clothianidin, imidacloprid, an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or any combination thereof. For example, the insecticide can comprise clothianidin or imidacloprid.

When the composition includes an insecticide, the insecticide can include an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or any combination thereof.

Also disclosed are kits that comprise a compound disclosed herein in one or more containers. The disclosed kits can optionally include agriculturally acceptable carriers and/or diluents. In one embodiment, a kit includes one or more other components, auxiliaries, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution.

A number of embodiments of the invention have been described. Nevertheless, it will be understood that various modifications may be made without departing from the spirit and scope of the invention. Accordingly, other embodiments are within the scope of the following claims.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Peptides and proteins are generally impermeable to the plant cell membrane. MTD4, an engineered human membrane translocating domain, can efficiently enter the cytosol of plant cells. Genetic fusion of MTD4 to HrpZ, a bacterial harpin, results in the effective delivery of HrpZ into tobacco and tomato plants, causing cell death and enhanced resistance to pathogens.

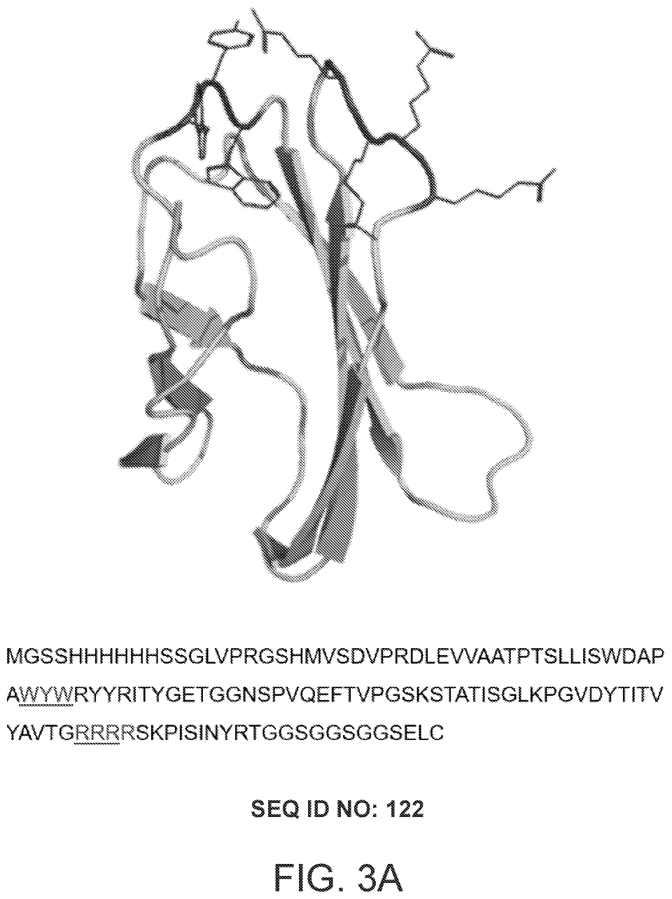

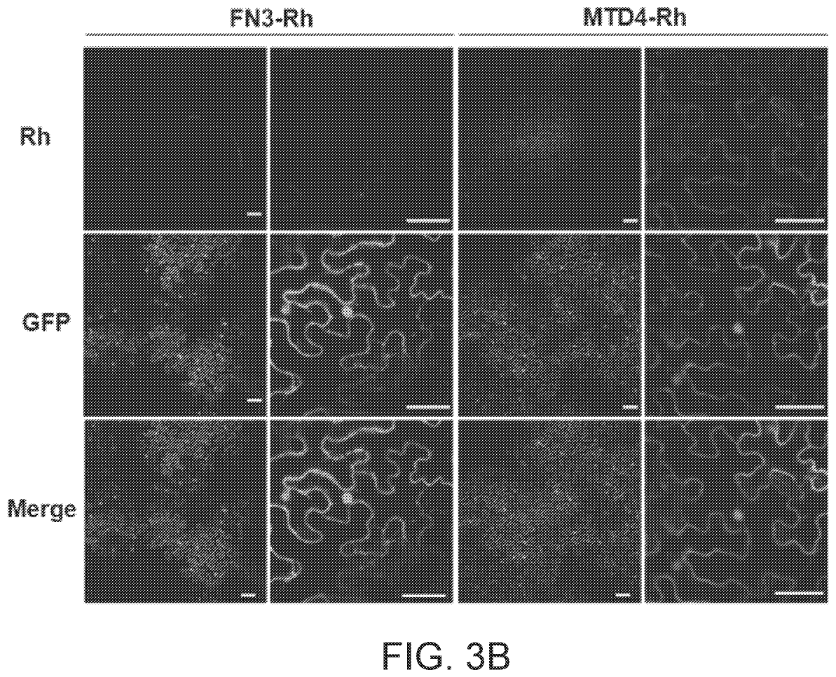

To explore whether MTD4 can permeate plant tissues and cells, MTD4 was expressed and purified as well as the parental protein (FN3) from Escherichia coli and labeled them with tetramethylrhodamine (TMR) (Liss, V., et al., Sci Rep 5 (2016)). The resulting proteins, MTD4^{TMR} and FN3^{TMR}, were sprayed on N. benthamiana leaves which had previously been infiltrated with an Agrobacterium expressing the green fluorescent protein (GFP). Two h post-spraying, confocal microscopic imaging revealed strong red fluorescence inside the cells of the N. benthamiana leaves treated with MTD4^{TMR} (FIG. 3B, right panel) but not those treated with FN3^{TMR} (FIG. 3B, left panel). As expected, GFP was expressed and localized in the cytosol of the cells. Colocalization of the green and red fluorescence signals at the cell membrane and inside the cytoplasm of the MTD4^{TMR}-sprayed leaves (FIG. 3B, right bottom panel) demonstrate that MTD4 efficiently enters the cytosol of N. benthamiana cells.

Harpin proteins are produced by Gram-negative bacteria and secreted through the type III secretion system, and function as elicitors of hypersensitive reaction (cell death), immune responses and plant growth enhancers (Choi, M., et al., Molecular Plant-Microbe Interactions 26, 1115-1122 (2013)). These unique characteristics render harpin proteins ideal defense activators and growth promoters of crops. Indeed, harpin-related products have been developed for crop plants over the past two decades. However, although effective in some applications, these products have not been widely used in crop production because the proteins do not efficiently penetrate plant tissues and therefore have very low bioavailability in foliar applications (Nadendla, S. R., et al., Carbohyd Polym 199, 11-19 (2018)).

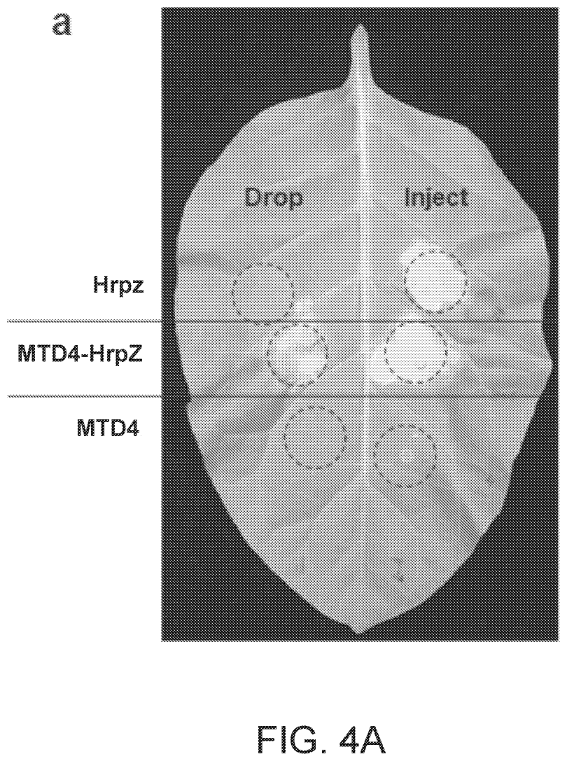

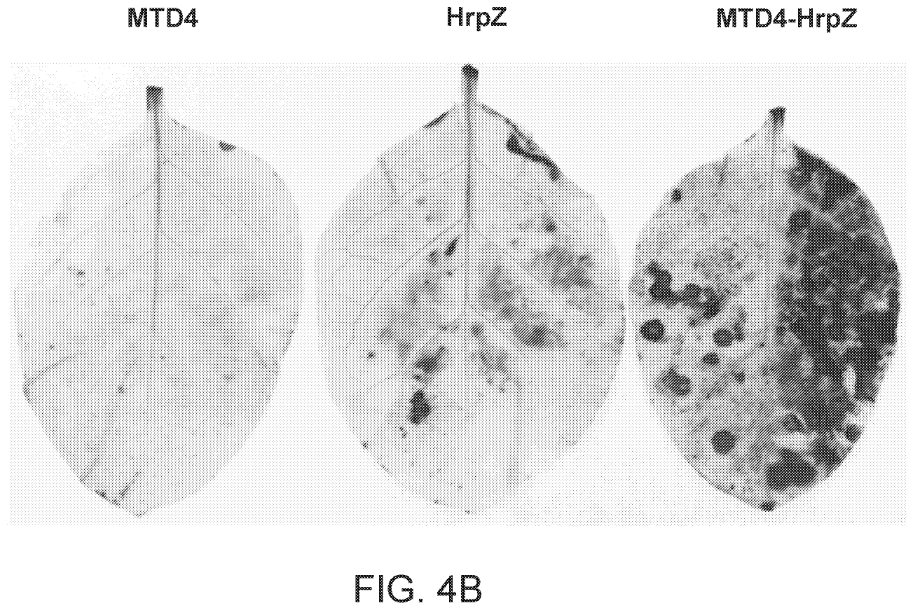

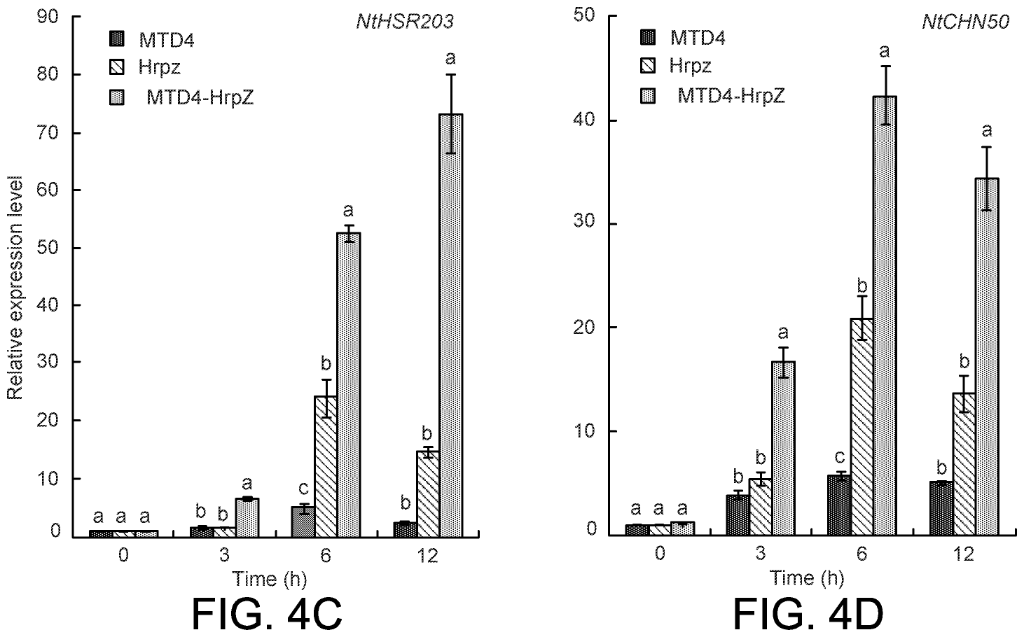

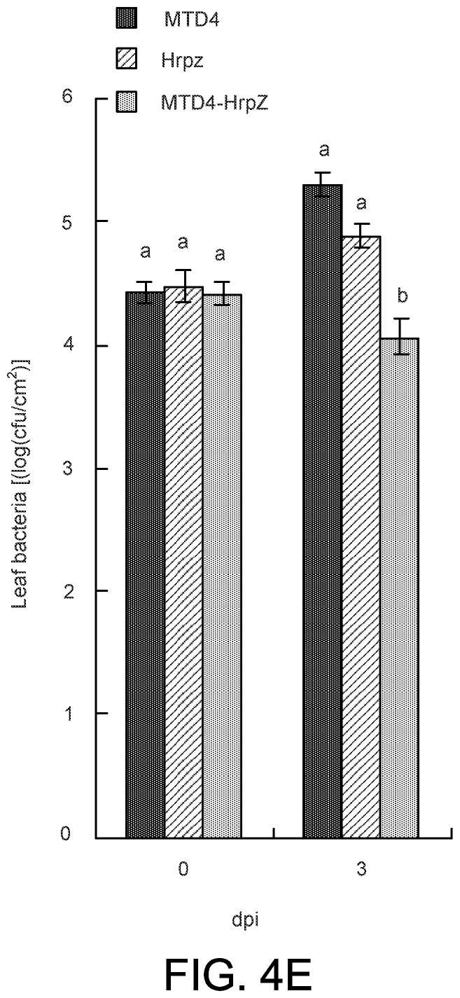

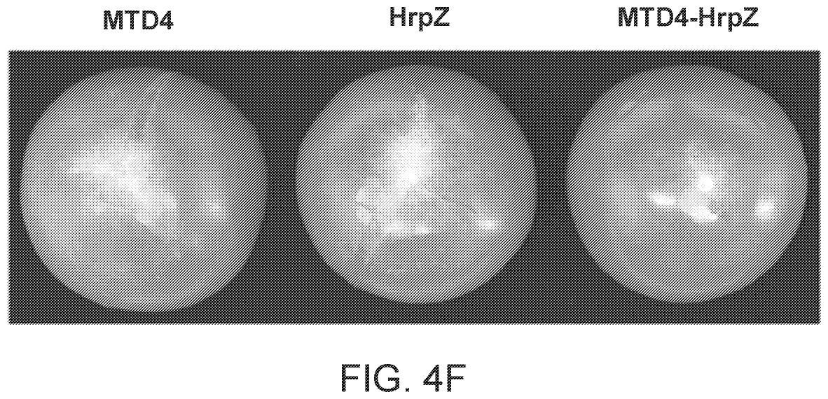

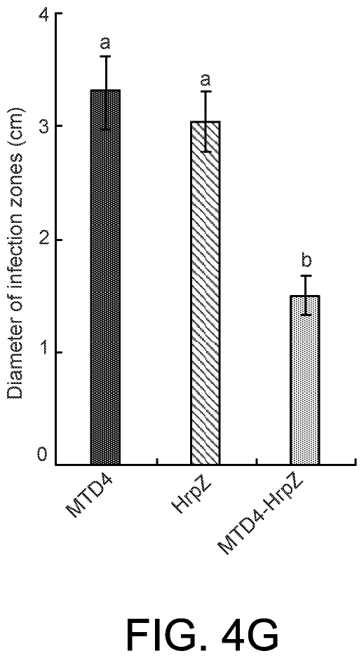

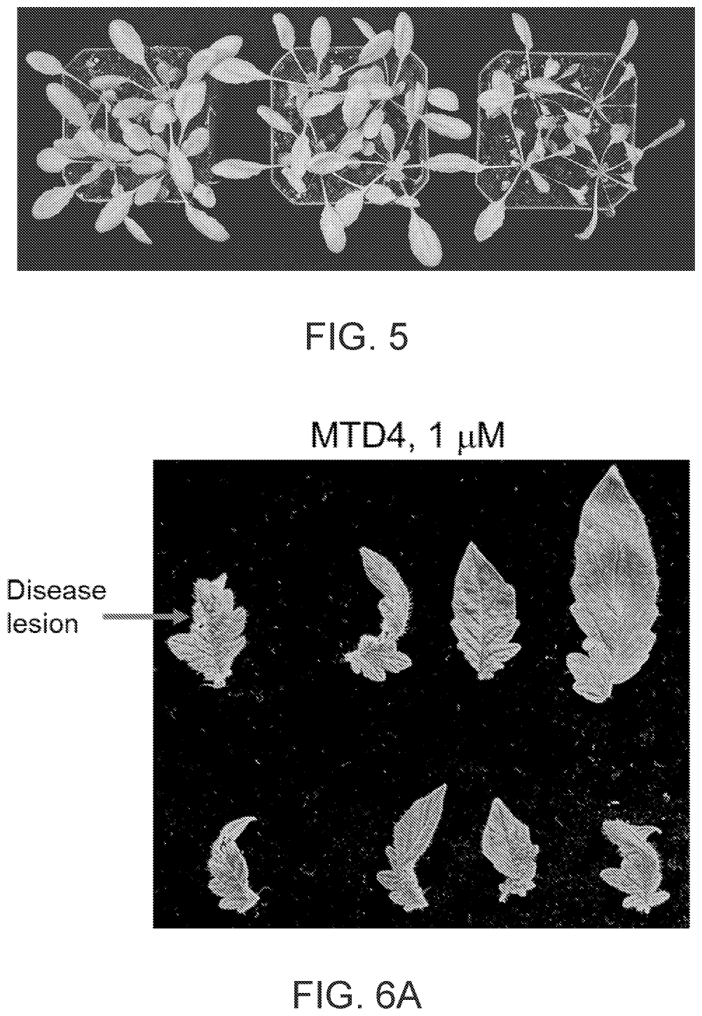

To test whether MTD4 can improve the permeability of cargo proteins into plant tissues, HrpZ (He, S. Y., et al., Cell 73, 1255-1266 (1993)) was genetically fused to the C-terminus of MTD4 and purified the MTD4-HrpZ fusion protein from E. coli. Treatment of N. tabacum leaves (by foliar application) for 24 h with 10 μM MTD4-HrpZ, but not HrpZ or MTD4, resulted in strong cell death in the leaves (FIG. 4A, left panel). In contrast, when N. tabacum leaves were infiltrated with MTD4-HrpZ, HrpZ, or MTD4, strong cell death was observed in either MTD4-HrpZ- or HrpZ-treated leaves (FIG. 4A, right panel), confirming the ability of HrpZ alone to induce cell death when delivered into the cytosolic space. It was also found that spraying Arabidopsis plants with MTD4-HrpZ caused strong cell death on the leaves 24 h after treatment (FIG. 5). These results demonstrate that MTD-HrpZ but not HrpZ can readily enter N. tabacum and Arabidopsis cells and cause cell death through foliar application.

Effect of MTD4-HrpZ on plant immunity. N. tabacum leaves were sprayed with MTD4-HrpZ and stained with 3, 3′-diaminobenzidine (DAB), which detects the level of reactive oxygen species (ROS) in the plant tissue. Treatment with MTD4-HrpZ resulted in significantly more ROS accumulation than those treated with HrpZ (FIG. 4B). In addition, qRT-PCR analysis showed that the expression of two pathogenesis-related genes, NtHSR203 and NtCHN50, in N. tabacum was significantly up-regulated as early as 3 h after spraying with MTD4-HrpZ, as compared to those sprayed with HrpZ (FIG. 4C). To test whether MTD4-HrpZ can enhance the resistance of N. tabacum to pathogenic bacterium, P. syringae pv. tomato (Pst) DC3000 hrcC−, N. tabacum plants were sprayed with MTD4-HrpZ, HrpZ, or MTD4 and measured the bacterial titer three days post-inoculation (dpi). Leaves sprayed with MTD4-HrpZ had about an order of magnitude fewer Pst bacteria than those treated with either HrpZ or MDT4 (FIG. 4D).

Botrytis cinerea is one of the most destructive fungal pathogens affecting numerous plant species including tomato (Cheung, N., et al., Pathogens 9, 923 (2020)). It was tested whether MTD4-HrpZ can protect tomato fruits against B. cinerea. Fresh tomatoes were sprayed with MTD4-HrpZ, HrpZ, or MTD4. After 24 h, the tomatoes were inoculated with hyphal blocks of B. cinerea on a wounded site. Three days post inoculation, the MTD4-HrpZ-treated tomatoes showed visibly less B. cinerea growth than those treated with either HrpZ or MTD4 (FIG. 4E). The lesion sizes in the MTD4-HrpZ-treated tomatoes were 2-fold smaller than those of the two controls (FIG. 4F).

It was demonstrated that the cell-penetrating peptide/protein (CPP) technology can significantly increases the cellular entry efficiency of a defense activator into plant cells. Cell-penetration efficiency of Lys-containing CPPs was greater in plants than animal cells, likely due to differences in lipid composition and surface charge of the membrane in these two cell types. MTD4 efficiently enters plant cells and significantly increases the cellular entry efficiency of HrpZ into plant cells. Since MTD4 may be genetically fused to any cargo peptide/protein and the resulting fusion protein may be readily produced in large-scale and cost-effectively through fermentation, it has great potential for delivering defense activators and biostimulants for foliar applications and seed treatments in crop production.

### Methods

Primers and strains. The primers used in this study are listed in Table 3. The strains and plasmids used in this study are listed in Table 4. All the gene constructs were confirmed by sequencing.

TABLE 3
List of primer sequences used in study.

| Primers | SEQ | | |
|---|---|---|---|
| (5′-3′ | ID | | |
| sequence) | NO: | Sequence | Purpose |
| HrpZ-f | 147 | ATAAATGAGCTCCAA | Primers for cloning |
| | | AGCCTATCCTTAAAT | of HrpZ downstream |
| | | AGTTCATCTTTACAA | of MTD4 |
| | | AC | |
| HrpZ-r | 148 | AATAATCTCGAGTTA | Primers for cloning |
| | | AGCCGCTGC | of HrpZ downstream |
| | | | of MTD4 |
| NTActin- | 149 | TCCTGATGGGCAAGT | Primers for qRT-PCR |
| qRT-F | | GATTAC | |
| NTActin- | 150 | TTGTATGTGGTCTCG | Primers for qRT-PCR |
| qRT-R | | TGGATTC | |
| NtHSR203- | 151 | TGGACCCTGTAACTG | Primers for qRT-PCR |
| qRT-F | | GTTCTG | |
| NtHSR203- | 152 | GAACGTGACAATCAA | Primers for qRT-PCR |
| qRT-R | | GACGG | |
| NtCHN50- | 153 | ATGCCAAGGAAAGGG | Primers for qRT-PCR |
| qRT-F | | ATTCTACA | |
| NtCHN50- | 154 | TGGGAGGTTTGGGCG | Primers for qRT-PCR |
| qRT-R | | AAGA | |

TABLE 4
List of strains and plasmids used in study.

| Strains and plasmids | Characteristics | Source |
|---|---|---|
| Strains | | |
| Escherichia coli DHSα | E. coli strain for plasmid construction | Lab collection |
| Escherichia coli BL21 | E. coli strain for plasmid construction | Lab collection |
| Agrobacterium tumefaciens GV3101 | Agrobacteria strain | Lab collection |
| Botrytis cinerea | Wild-type strain | Lab collection |
| P. syringae pv. tomato | Defective in the type III protein | Lab collection |
| DC3000 hrcC- | secretion system | |
| Plasmids | | |
| pYBA1132 | GFP tag protein expression vector | (Yan et al., 2012) |
| pet15b | Protein expression vector used in this study | GenScript |

Plant materials and growth conditions. Seeds of tobacco and Arabidopsis were sowed in soil pots and the plants were grown under an 8 h/16 h photoperiod at 25° C. in a growth room.

Design, Expression. and Purification of MTDs. Human Fibronectin Type III (FN3) domain was chosen as the scaffold for Membrane Translocation Domains (MTDs) (Koide, A., et al. (1998) The fibronectin type III domain as a scaffold for binding proteins. J. Mol. Biol. 284(4):1141-1151). FN3 is a small (90-100 aa), highly stable protein and has been widely used to develop monobodies that bind to target proteins with high affinity and specificity (Chandler, P. G., et al. (2020) Development and Differentiation in Monobodies Based on the Fibronectin Type 3 Domain. Cells 9(3):610). Previous studies have demonstrated that several loop regions of FN3 are tolerant to mutations (Steven, A., et al. (2012) Design of novel FN3 domains with high stability by a consensus sequence approach, Protein Engineering, Design and Selection, 25(3):107-117). Additionally, FN3 is free of any cysteine or disulfide bond and is thus stable in the intracellular environment. FN3 readily folds into its native form without any physical or chemical assistance and can be produced in Escherichia coli in high yields. Finally, FN3 is derived from an abundant human extracellular protein and is less likely to elicit any immune response.

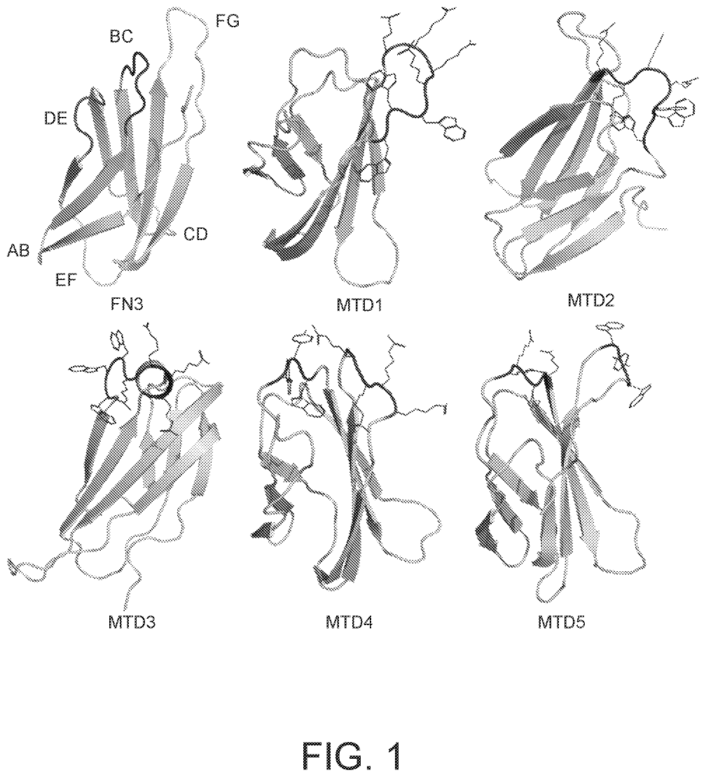

The BC, DE, and FG loops of FN3 have previously shown to be highly tolerant to sequence mutations. The GDSPAS (SEQ ID NO: 106) sequence of the FG loop was replaced with RRRWWW (SEQ ID NO: 104) to give MTD1 (Table 5). Together with an arginine residue already in the FG loop, this generates a putative CPP motif (R₄W₃) without altering the loop size. Similarly, the tetrapeptide AVTV of the BC loop was replaced with WWWRRR (SEQ ID NO: 105) to take advantage of the existing arginine in the loop to form a putative CPP, W₃R₄ (Table 5). The size of the BC loop in the resulting mutant, MTD2, is increased by 2 residues. To explore the possibility of grafting a CPP motif to the other end of FN3, the CPP motif R₄W₃ was substituted for the tripeptide NSP in the CD loop to give MTD3. To test the feasibility of grafting a CPP sequence into two different loops, a relatively hydrophobic tripeptide in the BC loop (VTV) was replaced with WYW and a hydrophilic motif in the FG loop (GDSPAS; SEQ ID NO: 106) with RRR to produce MTD4. Finally, MTD5 was generated by switching the WYW and RRR motifs of MTD4. Two mutants, MTD4a and MTD4b, which contain only half of the CPP motif in the BC and FG loops, respectively, were also generated to test the relative importance of the RRRR and WYW motifs. The WYW motif is more hydrophilic than WWW and has previously been reported as the “endosomal escape motif” of cell-permeable antibodies (Kim, J.-S., et al. (2016) Endosomal acidic pH-induced conformational changes of a cytosol-penetrating antibody mediate endosomal escape. J. Control. Rel. 235:165-175). The loop insertion mutants were analyzed by an online program, Phyre2, to predict their folded structures based on homology of sequences. All mutants maintained a similar overall folding to wild type FN3, with the CPP motifs displayed on their surfaces and constrained into the “cyclic” topology (FIG. 1).

To further improve the properties of MTD4 (e.g., cell entry efficiency, metabolic stability, and expression yield), the BC and FG loops of FN3 were replaced with different combinations of Y, W, A, and R residues to generate MTD6-10 (Table 5). The total cellular entry efficiency of MTD6-10 was assessed by labeling the MTDs at a unique C-terminal cysteine with tetramethylrhodamine-5-maleimide (TMR). HeLa cells were treated with the TMR-labeled proteins (5 μM) for 2 h and analyzed by live cell confocal microscopy. MTD7^{TMR} and MTD9^{TMR} showed similar uptake as MTD4^{TMR}, whereas MTD6^{TMR}, MTD8^{TMR} and MTD10^{TMR} showed much less cellular entry. Additionally, the isolated yields for MTD6-10 varied from 0.6 to 6.2 mg/L of E. coli cell culture (Table 4). Note that MTD4 and MTD6 differ only slightly in the BC loop sequence (“WYW” vs “YWW”) and yet have dramatic differences in the isolated yields (9.4 mg/L vs 0.9 mg/L) as well as the cell entry efficiency. Similarly, swapping the CPP motifs between the BC and FG loops of MTD4 resulted in a poorly expressed and much less active variant (MTD5 in Table 5). These results demonstrate that the proper folding/stability and high cellular entry efficiency of the MTDs require not only the presence of amphipathic CPP motifs but also their proper presentation on the protein surface.

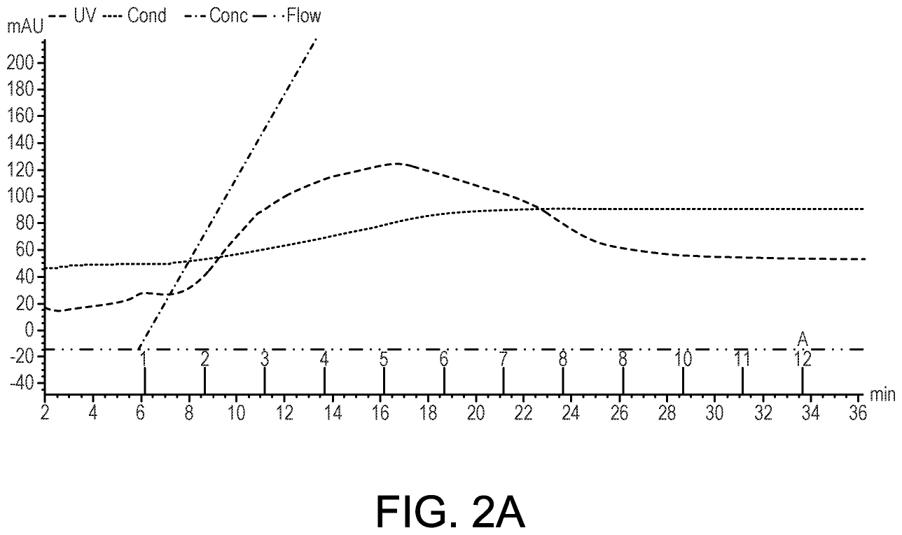

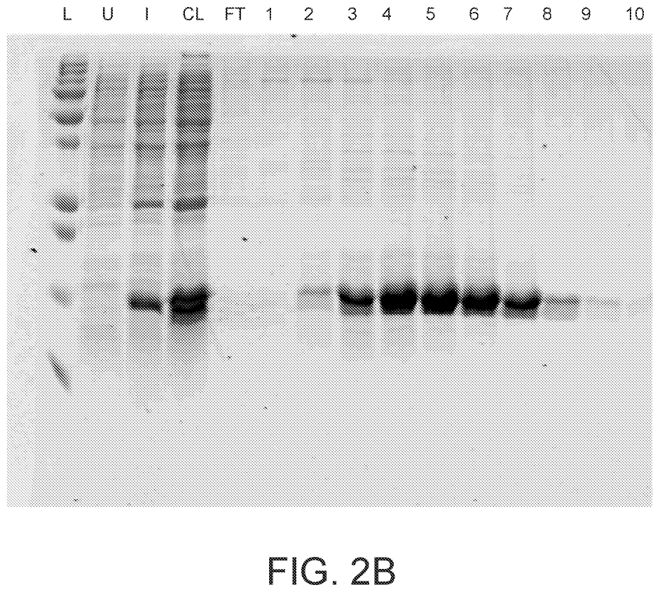

The DNA sequence coding for WT FN3 was chemically synthesized and ligated into prokaryotic expression vector pET-15b. To facilitate protein purification and genetic fusion with cargo proteins, a six-histidine tag and a thrombin cleavage site were added to the N-terminus of FN3, while a flexible linker sequence (GGSGGSGGS; SEQ ID NO: 107) followed by a recognition site for restriction endonuclease SacI and a cysteine was added to its C-terminus (Table 6). All loop insertion mutants were generated by one-step polymerase chain reaction (PCR) method (Qi, D., et al. (2008) A one-step PCR-based method for rapid and efficient site-directed fragment deletion, insertion, and substitution mutagenesis. J. Virol. Meth. 149:85-9020) and expressed in E. coli. Among the mutant proteins, MTD1 failed to produce significant amounts of soluble protein, whereas WT FN3 and MTD2-5 produced soluble proteins in good yields (Table 5). FIGS. 2A-2B show the expression and purification of MTD4 as an example. All proteins were purified to near homogeneity by metal affinity chromatography on a Ni-NTA column.

TABLE 5
Loop Sequences and Expression Yields of MTDs

| | Insertion | Original Sequence* | Mutant Sequence* | Quality of |
|---|---|---|---|---|
| Protein | Site | (SEQ ID NO:) | (SEQ ID NO:) | Expression |
| MTD1 | FG loop | TGRGDSPASSKPI (108) | TGRRRRWWWSKPI | No expression |
| | | | (111) | |
| MTD2 | BC loop | APAVTVRYY (109) | APWWWRRRRYY (112) | Soluble; yield 6.4 |
| | | | | mg/L |
| MTD3 | CD loop | GGNSPVQE (110) | GGRRRRWWWVQE | Soluble; yield 2 |
| | | | (113) | mg/L |
| MTD4 | BC loop | APAVTVRYY (109) | APAWYWRYY (114) | Soluble; yield 9.4 |
| | FG loop | TGRGDSPASSKPI (108) | TGRRRRSKPI (115) | mg/L |
| MTD5 | BC loop | APAVTVRYY (109) | APARRRRYY (116) | Soluble; yield 0.6 |
| | FG loop | TGRGDSPASSKPI (108) | TGWYWRSKPI (117) | mg/L |
| MTD6 | BC loop | APAVTVRYY (109) | APAYWWRYY (156) | Soluble; yield 0.9 |
| | FG loop | TGRGDSPASSKPI (108) | TGRRRRSKPI (157) | mg/L |
| MTD7 | BC loop | APAVTVRYY (109) | APAWWARYY (158) | Soluble; yield 2.5 |
| | FG loop | TGRGDSPASSKPI (108) | TGRRRRSKPI (159) | mg/L |
| MTD8 | BC loop | APAVTVRYY (109) | APAWWWRRYY (160) | Soluble; yield 3.6 |
| | FG loop | TGRGDSPASSKPI (108) | TGRRASSKPI (161) | mg/L |
| MTD9 | BC loop | APAVTVRYY (109) | APAWWARYY (162) | Soluble; yield 6.2 |
| | FG loop | TGRGDSPASSKPI (108) | TGWRRRRSKPI (163) | mg/L |
| MTD10 | BC loop | APAVTVRYY (109) | APAWWRRYY (164) | Soluble; yield 0.6 |
| | FG loop | TGRGDSPASSKPI (108) | TGRRWWSKPI (165) | mg/L |

*Underlined residues were deleted and bold-faced residues were inserted during mutagenesis.

Cloning, Expression, and Purification of MTDs. All loop insertion mutants were generated by one-step polymerase-chain reaction (PCR) method (Qi. D., id.). The peptide sequence (Table 6) for each construct was confirmed by sequencing the entire coding region of the plasmid DNA. Pilot-scale protein expression was carried out to check the levels of expression for Mutant proteins, all mutants were expressed in 5 mL E. coli BL21 (DE3) bacterial culture. The induction was carried out in presence of 0.25 mM IPTG at 37° C. The level of expression was checked by comparing total cell lysate before and after induction on SDS gel (FIGS. 2A-2B).

TABLE 6
Amino Acid Sequences of WT FN3, MTDs, and MTD-Cargo Fusions

| | SEQ | |
|---|---|---|
| | ID | |
| Protein | NO: | Sequence |
| FN3 | 118 | MGSSHHHHHHSSGLVPRGSHMVSDVPROLEVVAATPTSLLISWDAPAVTVRYYRITYGET |
| | | GGNSPVQEFTVPGSKSTATISGLKPGVDYTITVYAVTGRGDSPASSKPISINYRTGGSGG |
| | | SGGSELC |
| MTD1 | 119 | MGSSHHHHHHSSGLVPRGSHMVSDVPRDLEVVAATPTSLLISWDAPAVTVRYYRITYGET |
| | | GGNSPVQEFTVPGSKSTATISGLKPGVDYTITVYAVTGRRRRWWWSKPISINYRTGGSGG |
| | | SGGSELC |
| MTD2 | 120 | MGSSHHHHHHSSGLVPRGSHMVSDVPRDLEVVAATPTSLLISWDAPWWWRRRRYYRITYG |
| | | ETGGNSPVQEFTVPGSKSTATISGLKPGVDYTITVYAVTGRGDSPASSKPISINYRTGGS |
| | | GGSGGSELC |
| MTD3 | 121 | MGSSHHHHHHSSGLVPRGSHMVSDVPRDLEVVAATPTSLLISWDAPAVTVRYYRITYGET |
| | | GGRRRRWWWVQEFTVPGSKSTATISGLKPGVDYTITVYAVTGRGDSPASSKPISINYRTG |
| | | GSGGSGGSELC |
| MTD4 | 122 | MGSSHHHHHHSSGLVPRGSHMVSDVPRDLEVVAATPTSLLISWDAPAWYWRYYRITYGET |
| | | GGNSPVQEFTVPGSKSTATISGLKPGVDYTITVYAVTGRRRRSKPISINYRTGGSGGSGG |
| | | SELC |
| MTD4a | 123 | MGSSHHHHHHSSGLVPRGSHMVSDVPRDLEVVAATPTSLLISWDAPAWYWRYYRITYGET |
| | | GGNSPVQEFTVPGSKSTATISGLKPGVDYTITVYAVTGRGDSPASSKPISINYRTGGSGG |
| | | SGGSELC |
| MTD4b | 124 | MGSSHHHHHHSSGLVPRGSHMVSDVPRDLEVVAATPTSLLISWDAPAVTVRYYRITYGET |
| | | GGNSPVQEFTVPGSKSTATISGLKPGVDYTITVYAVTGRRRRSKPISINYRTGGSGGSGG |
| | | SELC |
| MTD5 | 125 | MGSSHHHHHHSSGLVPRGSHMVSDVPRDLEVVAATPTSLLISWDAPARRRRYYRITYGET |
| | | GGNSPVQEFTVPGSKSTATISGLKPGVDYTITVYAVTGWYWRSKPISINYRTGGSGGSGG |
| | | SELC |
| MTD4- | 126 | MGSSHHHHHHSSGLVPRGSHMVSDVPRDLEVVAATPTSLLISWDAPAWYWRYYRITYGET |
| PTP1B | | GGNSPVQEFTVPGSKSTATISGLKPGVDYTITVYAVTGRRRRSKPISINYRTGGSGGSGG |
| | | SELMEMEKEFEQIDKSGSWAAIYQDIRHEASDFPCRVAKLPKNKNRNRYRDVSPFDHSRI |
| | | KLHQEDNDYINASLIKMEEAQRSYILTQGPLPNTCGHFWEMVWEQKSRGVVMLNRVMEKG |
| | | SLKCAQYWPQKEEKEMIFEDTNLKLTLISEDIKSYYTVRQLELENLTTQETREILHFHYT |
| | | TWPDFGVPESPASFLNFLFKVRESGSLSPEHGPVVVHCSAGIGRSGTFCLADTCLLLMDK |
| | | RKDPSSVDIKKVLLEMRKFRMGLIQTADQLRFSYLAVIEGAKFIMGDSSVQDQWKELSHE |
| | | DLEPPPEHIPPPPRPPKRILEPHN |
| MTD4- | 127 | MGSSHHHHHHSSGLVPRGSHMVSDVPRDLEVVAATPTSLLISWDAPAWYWRYYRITYGET |
| NS1 | | GGNSPVQEFTVPGSKSTATISGLKPGVDYTITVYAVTGRRRRSKPISINYRTGGSGGSGG |
| | | SELGSVSSVPTKLEVVAATPTSLLISWOAPAVTVDYYVITYGETGGNSPVQKFEVPGSKS |
| | | TATISGLKPGVDYTITVYAWGWHGQVYYYMGSPISINYRT |
| MTD4- | 128 | MGSSHHHHHHSSGLVPRGSHMVSDVPRDLEVVAATPTSLLISWDAPAWYWRYYRITYGET |
| RBDV | | GGNSPVQEFTVPGSKSTATISGLKPGVDYTITVYAVTGRRRRSKPISINYRTGGSGGSGG |
| | | SELKTSNTIRVLLPNQEWTVVKVRNGMSLHDSLMKALKRHGLQPESSAVFRLLHEHKGKK |
| | | ARLDWNTDAASLIGEELQVDFLDHVPLTTHNFARKTFLKLGIHRD |
| MTD4- | 166 | MGSSHHHHHHSSGLVPRGSHMVSDVPRDLEVVAATPTSLLISWDAPAWYWRYYRITYGET |
| SEP | | GGNSPVQEFTVPGSKSTATISGLKPGVDYTITVYAVTGRRRRSKPISINYRTGGSGGSGG |
| | | SELSKGEELFTGVVPILVELDGDVNGHKFSVSGEGEGDATYGKLTLKFICTTGKLPVPWP |
| | | TLVTTLTYGVQCFSRYPDHMKRHDFFKSAMPEGYVQERTIFFKDDGNYKTRAEVKFEGOT |
| | | LVNRIELKGIDFKEDGNILGHKLEYNYNDHQVYIMADKQKNGIKANFKIRHNIEDGGVQL |
| | | ADHYQQNTPIGDGPVLLPDNHYLFTTSTLSKDPNEKRDHMVLLEFVTAAGITHGMDELYK |
| MTD6 | 167 | MGSSHHHHHHSSGLVPRGSHMVSDVPRDLEVVAATPTSLLISWDAPAYWWRYYRITYGET |
| | | GGNSPVQEFTVPGSKSTATISGLKPGVDYTITVYAVTGRRRRSKPISINYRTGGSGGSGG |
| | | SELC |
| MTD7 | 168 | MGSSHHHHHHSSGLVPRGSHMVSDVPRDLEVVAATPTSLLISWDAPAWWARYYRITYGET |
| | | GGNSPVQEFTVPGSKSTATISGLKPGVDYTITVYAVTGRRRRSKPISINYRTGGSGGSGG |
| | | SELC |
| MTD8 | 169 | MGSSHHHHHHSSGLVPRGSHMVSDVPRDLEVVAATPTSLLISWDAPAWWWRRYYRITYGE |
| | | TGGNSPVQEFTVPGSKSTATISGLKPGVDYTITVYAVTGRRASSKPISINYRTGGSGGSG |
| | | GSELC |
| MTD9 | 170 | MGSSHHHHHHSSGLVPRGSHMVSDVPRDLEVVAATPTSLLISWDAPAWWARYYRITYGET |
| | | GGNSPVQEFTVPGSKSTATISGLKPGVDYTITVYAVTGWRRRRSKPISINYRTGGSGGSG |
| | | GSELC |
| MTD10 | 171 | MGSSHHHHHHSSGLVPRGSHMVSDVPRDLEVVAATPTSLLISWDAPAWWRRYYRITYGET |
| | | GGNSPVQEFTVPGSKSTATISGLKPGVDYTITVYAVTGRRWWSKPISINYRTGGSGGSGG |
| | | SELC |

The large-scale expression conditions were the same as used for small scale, E. coli cells were centrifuged and stored at −80° C. These cells were lysed using lysis buffer (50 mL of wash buffer, 0.2 mg/mL lysozyme, 2 mM P-mercaptoethanol, 2 mM PMSF, 2 tablets of Roche complete protease inhibitor cocktail). After homogeneously resuspending the cell palate in lysis buffer the cells were sonicated (Amp. 70%) twice. The crude cell lysate was centrifuged (12000 g for 20 min) and the soluble cell lysate was collected. Protein purification was carried out by using fast protein liquid chromatography (FPLC) and the soluble cell lysate was loaded onto a Ni-NTA column (with 15 mM imidazole). The column was exhaustively washed with wash buffer (50 mM Tris, pH 7.4, 300 mM NaCl, 5% glycerol and 50 mM imidazole). Protein was eluted with wash buffer containing a linear gradient of 50-500 mM imidazole (pH 7.4) over 30 min.

MTD4 Plasmid construction. The DNA sequences coding for the tenth FN3 domain and HrpZ were chemically synthesized and ligated with prokaryotic expression vector pET-15b (MilliporeSigma), which had been linearized by using restriction endonucleases NdeI and XhoI. The cloning procedure also resulted in the addition of a flexible linker sequence, (GGS)₃, a restriction site (SacI), and a cysteine residue to the C-terminus of FN3. Site-directed mutagenesis of the FN3 to generate plasmid pET-15b-MTD4 was described previously (Qi, D.; Scholthof, K.-B. G., A one-step PCR-based method for rapid and efficient site-directed fragment deletion, insertion, and substitution mutagenesis. J. Virol. Methods 2008, 149 (1), 85-90). The HrpZ gene was amplified by PCR using plasmid DNA as template and primers containing SacI and XhoI restriction sites at the 5′ and 3′ terminus of the HrpZ sequence, respectively (Table 3). The PCR product was digested with SacI and XhoI restriction enzymes and ligated into plasmid pET-15b-MTD4 linearized with the same two enzymes. All the constructs comprise a six-histidine tag at the N-terminus for facile purification. The authenticity of the DNA construct was confirmed by restriction mapping and sequencing of the entire coding sequence.

HrpZ protein sequence
SEQ ID NO: 146
MQSLSLNSSSLQTPAMALVLVRPEAETTGSTSSKALQEVVVKLAEELMRN
GQLDDSSPLGKLLAKSMAADGKAGGGIEDVIAALDKLIHEKLGDNFGASA
DSASGTGQQDLMTQVLNGLAKSMLDDLLTKQDGGTSFSEDDMPMLNKIAQ
FMDDNPAQFPKPDSGSWVNELKEDNFLDGDETAAFRSALDIIGQQLGNQQ
SDAGSLAGTGGGLGTPSSFSNNSSVMGDPLIDANTGPGDSGNTRGEAGQL
IGELIDRGLQSVLAGGGLGTPVNTPQTGTSANGGQSAQDLDQLLGGLLLK
GLEATLKDAGQTGTDVQSSAAQIATLLVSTLLQGTRNQAAA
HrpZ DNA sequence based on
GenBank acc. #: HM358042.1
(SEQ ID NO: 155)
ATGCAGAGTCTCAGTCTTAACAGCAGCTCGCTGCAAACCCCGGCAATGGC
CCTTGTCCTGGTACGTCCTGAAGCCGAGACGACTGGCAGTACGTCGAGCA
AGGCGCTTCAGGAAGTTGTCGTGAAGCTGGCCGAGGAACTGATGCGCAAT
GGTCAACTCGACGACAGCTCGCCATTGGGAAAACTGTTGGCCAAGTCGAT
GGCCGCAGATGGCAAGGCGGGCGGCGGTATTGAGGATGTCATCGCTGCGC
TGGACAAGCTGATCCATGAAAAGCTCGGTGACAACTTCGGCGCGTCTGCG
GACAGCGCCTCGGGTACCGGACAGCAGGACCTGATGACTCAGGTGCTCAA
TGGCCTGGCCAAGTCGATGCTCGATGATCTTCTGACCAAGCAGGATGGCG
GGACAAGCTTCTCCGAAGACGATATGCCGATGCTGAACAAGATCGCGCAG
TTCATGGATGACAATCCCGCACAGTTTCCCAAGCCGGACTCGGGCTCCTG
GGTGAACGAACTCAAGGAAGACAACTTCCTTGATGGCGACGAAACGGCTG
CGTTCCGTTCGGCACTCGACATCATTGGCCAGCAACTGGGTAATCAGCAG
AGTGACGCTGGCAGTCTGGCAGGGACGGGTGGAGGTCTGGGCACTCCGAG
CAGTTTTTCCAACAACTCGTCCGTGATGGGTGATCCGCTGATCGACGCCA
ATACCGGTCCCGGTGACAGCGGCAATACCCGTGGTGAAGCGGGGCAACTG
ATCGGCGAGCTTATCGACCGTGGCCTGCAATCGGTATTGGCCGGTGGTGG
ACTGGGCACACCCGTAAACACCCCGCAGACCGGTACGTCGGCGAATGGCG
GACAGTCCGCTCAGGATCTTGATCAGTTGCTGGGCGGCTTGCTGCTCAAG
GGCCTGGAGGCAACGCTCAAGGATGCCGGGCAAACAGGCACCGACGTGCA
GTCGAGCGCTGCGCAAATCGCCACCTTGCTGGTCAGTACGCTGCTGCAAG
GCACCCGCAACCAGGCTGCTGCCTGA

Expression and purification of recombinant proteins. E. coli BL21 (DE3) cells transformed with the proper plasmid were grown in Luria-Bertani (LB) media supplemented with 75 μg/mL ampicillin at 37° C. When OD₆₀₀ reached 0.6, the cells were induced by the addition of 0.25 mM isopropyl β-d-1-thiogalactopyranoside (IPTG) at 37° C. for 6 h. The cells were pelleted by centrifugation (4000 g for 30 min) and resuspended in lysis buffer (50 mL of wash buffer per 1 L of cell culture, 0.2 mg/mL lysozyme, 2 mM β-mercaptoethanol, 2 mM PMSF, 2 tablets of Roche complete protease inhibitor cocktail). The cells were briefly sonicated and the crude cell lysate was centrifuged (12000 g for 20 min). The supernatant was loaded onto a 5-mL HisTrap FF nickel affinity column (Cytiva). The column was exhaustively washed with wash buffer (20 mM Tris, 300 mM NaCl, and 5% glycerol, adjusted pH 7.4) and wash buffer plus 50 mM imidazole. Protein was eluted with wash buffer containing a linear gradient of 50-500 mM imidazole. The purity of eluted fractions was assessed by SDS-PAGE and pure protein fractions were combined, concentrated in centrifugal filter units (Millipore), and dialyed against wash buffer. Protein concentration was determined by the Bradford assay. Purified proteins were supplemented with 30% glycerol, aliquoted, quickly frozen in isopropanol bath and stored at −80° C.

Fluorescent labelling of proteins. FN3 and MTD4 stock solutions were passed through a desalting spin column (Bio-rad) to remove β-mercaptoethanol. The proteins were treated with 1 mM tris(2-carboxyethyl)phosphine (TCEP) to ensure that the C-terminal cysteine is in the reduced form. Tetramethylrhodamine-5-maleimide (TMR; 8 equivalents) was dissolved in N, N-dimethylformamide (DMF) and slowly added to the protein solution, and the reaction was allowed to proceed for 2 h at room temperature. Excess dye was removed by passing the reaction mixture through a desalting spin column. The stoichiometry of dye labeling was estimated by comparing the absorbances at 280 nm (λₘₐₓ of protein) and 544 nm (λₘₐₓ of TMR) on a nanodrop spectrophotometer.

Subcellular localization assay. pYBA1132 plasmid containing green fluorescent protein (GFP) tag was transformed into the A. tumefaciens strain GV3101 (Yan, X., et al. Mol. Plant Breed 10, 371-379 (2012)) before it was used to infiltrate N. benthamiana leaves. Two days after the infiltration, MTD4-Rh or FN3-Rh (10 μM) was sprayed on N. benthamiana leaves and the treated leaves were incubated in the dark at 23° C. The protein solution on the surface of N. benthamiana leaves was washed thrice with H₂O₂ two h after spraying. Red and green fluorescence were observed using a confocal laser scanning microscopy with excitation at 488 nm and 514 nm, respectively.

DAB staining. The tobacco leaves were submerged in the 3,3′-diaminobenzidine (DAB) staining buffer (10 mM MES, pH 6.5 and 1 mg/mL DAB) at 23° C. for 18 h. The leaves were then transferred to 90% (v/v) ethanol at 65° C. until they became clear.

qRT-PCR. Total RNA was isolated from tobacco leaves using the TRIzol Reagent following the manufacturer's instructions. qRT-PCR was performed to quantify the expression level of pathogenesis-related genes. The gene transcript levels were normalized to the expression of the reference gene NtActin. The primers used to detect the expression of NtHSR203, NtCHN50 and NtActin were as previously described (Zhang, C., et al. J Exp Bot 70, 5407-5421 (2019)). The experiment was performed with three biological replicates.

Bacterial inoculation. Pseudomonas syringae pv. tomato (Pst) DC3000 hrcC− strain, which is deficient in type III secretion systems, was used for bacterial inoculation assays as previously described with minor modifications (Zhang, X., et al., Plant Mol Biol 90, 19-31 (2016)). Bacteria were cultured at 28° C. on King's medium containing 25 mg/L rifampicin. Fresh overnight bacterial culture was collected and centrifuged at 4000 rpm for 5 min and washed twice with 10 mM MgCl₂. The OD600 of the suspension was determined by spectrometry and further diluted to 0.2 with 10 mM MgCl₂ and infiltrated into four-week-old N. tabacum leaves. Samples were collected at 0 and 3 dpi. The samples were triturated in 10 mM MgCl₂ and were serially diluted on King's medium containing 25 mg/L rifampicin. The number of colonies formed was counted two days after spreading on the plates.

Fungal Inoculation. Tomatoes were disinfected with 75% (v/v) ethanol and sprayed with 10 μM MTD4-HrpZ, HrpZ, or MTD4. Before inoculation with B. cinerea, tomato fruits were wounded by making a small cut with a sharp blade. B. cinerea was cultured on the potato dextrose agar (PDA) medium at 28° C. in an incubator. B. cinerea hyphal blocks were placed on the wounded site 24 h after spraying with protein solutions. Disease symptoms were evaluated 3 dpi.

Statistical analysis. Multiple comparisons were performed using GraphPad Prism version 8.00 for Windows with one-way ANOVA followed by Dunnett's multiple comparisons test.

### Example 2: MTD4-HrpZ for Disease Control in Crop Plants

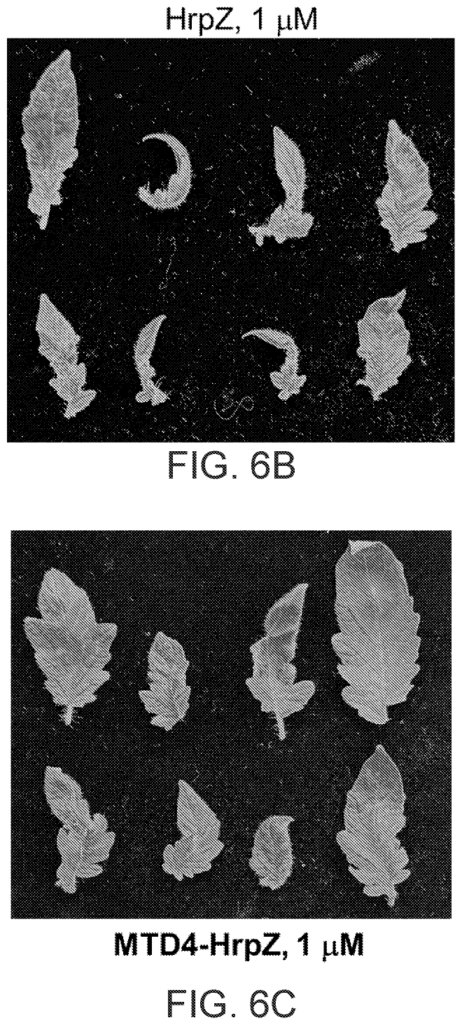

To test the application of MTD4-HrpZ for disease control in crop plants, tomato plant was used because it is one of the most popular vegetables in the US. First the optimum concentration of MTD4-HrpZ was determined on tomato plants that induce sufficient defense responses to the bacterial speck pathogen Pseudomonas syringae pv. tomato (Pst) DC3000, but does not cause cell death. Tomato seedlings (cultivar OH88119) were growing in a growth chamber with 26° C. in the day and 22° C. at night and a 12-hr light/dark cycle. Four-week-old plants were sprayed with different concentrations of MTD4-HrpZ, HrpZ and MTD4 and 24 h later were inoculated with Pst. Disease symptoms were observed one week after inoculation. Spraying tomato plants with 1 μM MTD4-HrpZ inhibited Pst invasion effectively, whereas MTD4 or HrpZ did not (FIGS. 6A-6C).

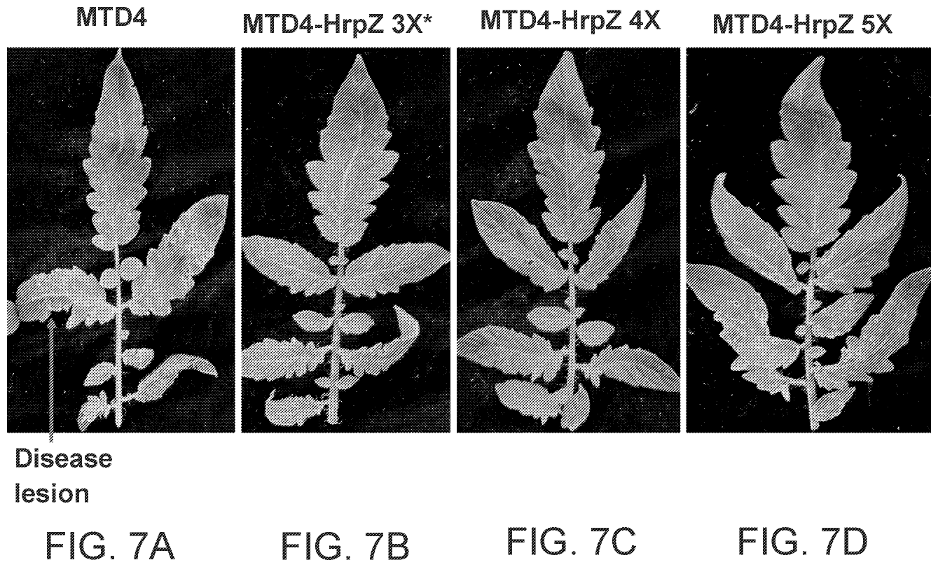

In addition, the effectiveness of MTD4-HrpZ was tested against Xanthomonas euvesicatoria pv. perforans (Xep), the causal agent of the bacterial spot disease. Similar to the results with Pst, application of 1 μM MTD4-HrpZ provided strong protection against Xep infection in 4-week-old tomato plants. Moreover, crude bacterial cell lysates derived from MTD4-HrpZ-overproducing E. coli cells were also effective for protecting 4-week-old tomato plants against bacterial pathogens. A 3× dilution of the crude cell lysate protected the tomato plant from Xep infection (FIGS. 7A-7D).

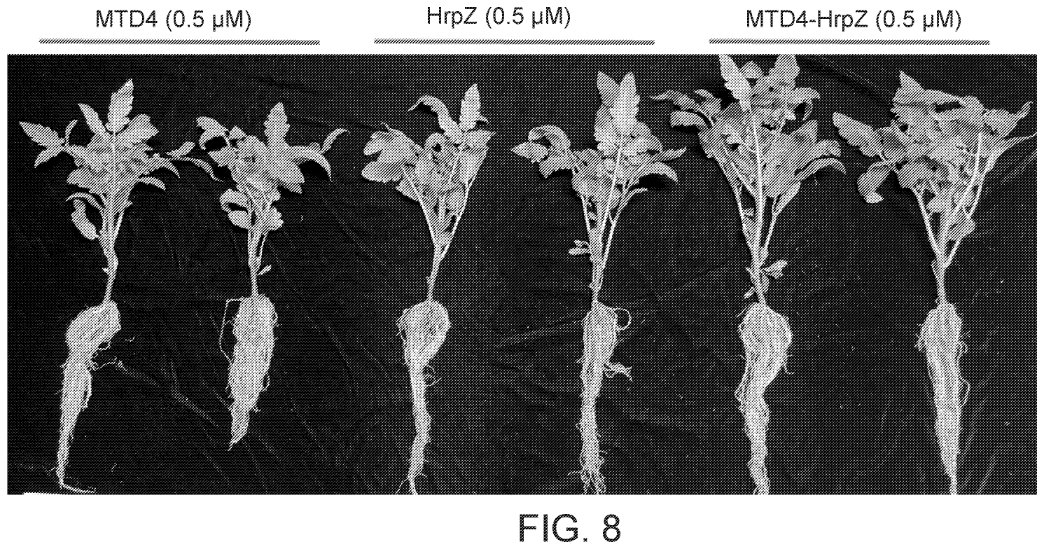

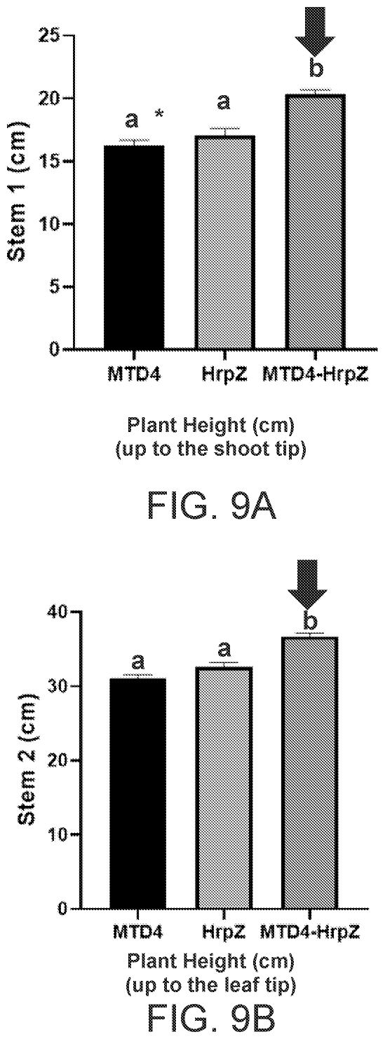

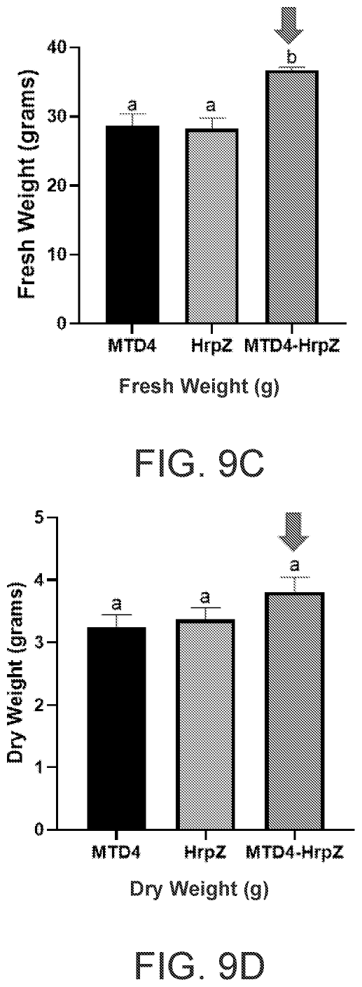

To determine whether MTD4-HrpZ promotes plant growth, tomato seedlings were sprayed with 0.5 μM MTD4-HrpZ twice (6 and 16 days after germination). Treatment with MTD4-HrpZ increased the plant height and fresh weight by 15-20% compared to the MTD4- or HrpZ-treated controls (FIGS. 8 and 9A-9D).

Collectively, the above results clearly demonstrate that MTD4 markedly increases the entry of HrpZ into plant cells during foliar applications resulting in enhanced immunity to bacterial pathogens and growth promotion.

SEQUENCES

| SEQ ID NO: | SEQUENCE |
|---|---|
| SEQ ID NO: 1 | FΦRRR |
| SEQ ID NO: 2 | FΦRRRC |
| SEQ ID NO: 3 | FΦRRRU |
| SEQ ID NO: 4 | RRRΦF |
| SEQ ID NO: 5 | RRRRΦF |
| SEQ ID NO: 6 | FΦRRRR |
| SEQ ID NO: 7 | FϕrRrR |
| SEQ ID NO: 8 | FϕrRrR |
| SEQ ID NO: 9 | FΦRRRR |
| SEQ ID NO: 10 | fΦRrRr |
| SEQ ID NO: 11 | RRFRΦR |
| SEQ ID NO: 12 | FRRRRΦ |
| SEQ ID NO: 13 | FRFRΦR |
| SEQ ID NO: 14 | RRΦFRR |
| SEQ ID NO: 15 | CRRRRFW |
| SEQ ID NO: 16 | FfΦRrRr |
| SEQ ID NO: 17 | FFΦRRRR |
| SEQ ID NO: 18 | RFRFRΦR |
| SEQ ID NO: 19 | URRRRFW |
| SEQ ID NO: 20 | CRRRRFW |
| SEQ ID NO: 21 | FΦRRRRQK |
| SEQ ID NO: 22 | FΦRRRRQC |
| SEQ ID NO: 23 | fΦRrRrR |
| SEQ ID NO: 24 | FΦRRRRR |
| SEQ ID NO: 25 | RRRRΦFDΩC |
| SEQ ID NO: 26 | FΦRRR |
| SEQ ID NO: 27 | FWRRR |
| SEQ ID NO: 28 | RRRΦF |
| SEQ ID NO: 29 | RRRWF |
| SEQ ID NO: 30 | FΦRRRR |
| SEQ ID NO: 31 | FFRRR |
| SEQ ID NO: 32 | FFrRr |
| SEQ ID NO: 33 | FFRrR |
| SEQ ID NO: 34 | FRFRR |
| SEQ ID NO: 35 | FRRFR |
| SEQ ID NO: 36 | FRRRF |
| SEQ ID NO: 37 | GΦRRR |
| SEQ ID NO: 38 | FFFRA |
| SEQ ID NO: 39 | FFFRR |
| SEQ ID NO: 40 | FFRRRR |
| SEQ ID NO: 41 | FRRFRR |
| SEQ ID NO: 42 | FRRRFR |
| SEQ ID NO: 43 | RFFRRR |
| SEQ ID NO: 44 | RFRRFR |
| SEQ ID NO: 45 | FRFRRR |
| SEQ ID NO: 46 | FFFRRR |
| SEQ ID NO: 47 | FFRRRF |
| SEQ ID NO: 48 | FRFFRR |
| SEQ ID NO: 49 | RRFFFR |
| SEQ ID NO: 50 | FFRFRR |
| SEQ ID NO: 51 | FFRRFR |
| SEQ ID NO: 52 | FRRFFR |
| SEQ ID NO: 53 | FRRFRF |
| SEQ ID NO: 54 | FRFRFR |
| SEQ ID NO: 55 | RFFRFR |
| SEQ ID NO: 56 | GΦRRRR |
| SEQ ID NO: 57 | FFFRRRR |
| SEQ ID NO: 58 | RFFRRRR |
| SEQ ID NO: 59 | RRFFRRR |
| SEQ ID NO: 60 | RFFFRRR |
| SEQ ID NO: 61 | RRFFFRR |
| SEQ ID NO: 62 | FFRRFRR |
| SEQ ID NO: 63 | FFRRRRF |
| SEQ ID NO: 64 | FRRFFRR |
| SEQ ID NO: 65 | FFFRRRRR |
| SEQ ID NO: 66 | FFFRRRRRR |
| SEQ ID NO: 67 | FΦRrRr |
| SEQ ID NO: 68 | XXRRRR |
| SEQ ID NO: 69 | FfFRrR |
| SEQ ID NO: 70 | fFfrRr |
| SEQ ID NO: 71 | fFfRrR |
| SEQ ID NO: 72 | FfFrRr |
| SEQ ID NO: 73 | fFφrRr |
| SEQ ID NO: 74 | fΦfrRr |
| SEQ ID NO: 75 | φFfrRr |
| SEQ ID NO: 76 | FΦrRr |
| SEQ ID NO: 77 | fΦrRr |
| SEQ ID NO: 78 | Ac-Lys-fFRrRrD |
| SEQ ID NO: 79 | Ac-Dap-fFRrRrD |
| SEQ ID NO: 80 | Pip-Nal-Arg-Glu-arg-arg-glu |
| SEQ ID NO: 81 | Pip-Nal-Arg-Arg-arg-arg-glu |
| SEQ ID NO: 82 | Pip-Nal-Nal-Arg-arg-arg-glu |
| SEQ ID NO: 83 | Pip-Nal-Nal-Arg-arg-arg-Glu |
| SEQ ID NO: 84 | Pip-Nal-Phe-Arg-arg-arg-glu |
| SEQ ID NO: 85 | Pip-Nal-Phe-Arg-arg-arg-Glu |
| SEQ ID NO: 86 | Pip-Nal-phe-Arg-arg-arg-glu |
| SEQ ID NO: 87 | Pip-Nal-phe-Arg-arg-arg-Glu |
| SEQ ID NO: 88 | Pip-Nal-nal-Arg-arg-arg-Glu |
| SEQ ID NO: 89 | Pip-Nal-nal-Arg-arg-arg-glu |
| SEQ ID NO: 90 | [P-Cha-r-Cha-r-Cha-r-Cha-r-G]^{d} |
| SEQ ID NO: 91 | Tm(SvP-F₂Pmp-H)-Dap-(FΦRRRR-Dap)]^{f} |
| SEQ ID NO: 92 | [Tm(a-Sar-D-pThr-Pip-ΦRAa)-Dap-(FΦRRRR-Dap)]^{f} |
| SEQ ID NO: 93 | [CRRSRRGCGRRSRRCG]^{g} |
| SEQ ID NO: 94 | [K(Dod)RRRR] |
| SEQ ID NO: 95 | LKKLCKLLKKLCKLAG |
| SEQ ID NO: 96 | RRRR-[KRRRE]^{c} |
| SEQ ID NO: 97 | RRR-[KRRRRE]^{c} |
| SEQ ID NO: 98 | RR-[KRRRRRE]^{c} |
| SEQ ID NO: 99 | R-[KRRRRRRE]^{c} |
| SEQ ID NO: 100 | [CRCRCRCR] |
| SEQ ID NO: 101 | [Pra-LRKRLRKFRN-AzK]^{h} |
| SEQ ID NO: 102 | T-Dap-[Dap-Dap-f-L-Dap-Dap-T] |
| SEQ ID NO: 103 | T-Agp-[Dap-Agp-f-L-Agp-Agp-T] |
| SEQ ID NO: 104 | RRRWWW |
| SEQ ID NO: 105 | WWWRRR |
| SEQ ID NO: 106 | GDSPAS |
| SEQ ID NO: 107 | GGSGGSGGS |
| SEQ ID NO: 108 | TGRGDSPASSKPI |
| SEQ ID NO: 109 | APAVTVRYY |
| SEQ ID NO: 110 | GGNSPVQE |
| SEQ ID NO: 111 | TGRRRRWWWSKPI |
| SEQ ID NO: 112 | APWWWRRRRYY |
| SEQ ID NO: 113 | GGRRRRWWWVQE |
| SEQ ID NO: 114 | APAWYWRYY |
| SEQ ID NO: 115 | TGRRRRSKPI |
| SEQ ID NO: 116 | APARRRRYY |
| SEQ ID NO: 117 | TGWYWRSKPI |
| SEQ ID NO: 118 | MGSSHHHHHHSSGLVPRGSHMVSDVPRDLEVVAATPTSLLISWDAPAVTVRYYR |
| | ITYGETGGNSPVQEFTVPGSKSTATISGLKPGVDYTITVYAVTGRGDSPASSKP |
| | ISINYRTGGSGGSGGSELC |
| SEQ ID NO: 119 | MGSSHHHHHHSSGLVPRGSHMVSDVPRDLEVVAATPTSLLISWDAPAVTVRYYR |
| | ITYGETGGNSPVQEFTVPGSKSTATISGLKPGVDYTITVYAVTGRRRRWWWSKP |
| | ISINYRTGGSGGSGGSELC |
| SEQ ID NO: 120 | MGSSHHHHHHSSGLVPRGSHMVSDVPRDLEVVAATPTSLLISWDAPWWWRRRRY |
| | YRITYGETGGNSPVQEFTVPGSKSTATISGLKPGVDYTITVYAVTGRGDSPASS |
| | KPISINYRTGGSGGSGGSELC |
| SEQ ID NO: 121 | MGSSHHHHHHSSGLVPRGSHMVSDVPRDLEVVAATPTSLLISWDAPAVTVRYYR |
| | ITYGETGGRRRRWWWVQEFTVPGSKSTATISGLKPGVDYTITVYAVTGRGDSPA |
| | SSKPISINYRTGGSGGSGGSELC |
| SEQ ID NO: 122 | MGSSHHHHHHSSGLVPRGSHMVSDVPRDLEVVAATPTSLLISWDAPAWYWRYYR |
| | ITYGETGGNSPVQEFTVPGSKSTATISGLKPGVDYTITVYAVTGRRRRSKPISI |
| | NYRTGGSGGSGGSELC |
| SEQ ID NO: 123 | MGSSHHHHHHSSGLVPRGSHMVSDVPRDLEVVAATPTSLLISWDAPAWYWRYYR |
| | ITYGETGGNSPVQEFTVPGSKSTATISGLKPGVDYTITVYAVTGRGDSPASSKP |
| | ISINYRTGGSGGSGGSELC |
| SEQ ID NO: 124 | MGSSHHHHHHSSGLVPRGSHMVSDVPRDLEVVAATPTSLLISWDAPAVTVRYYR |
| | ITYGETGGNSPVQEFTVPGSKSTATISGLKPGVDYTITVYAVTGRRRRSKPISI |
| | NYRTGGSGGSGGSELC |
| SEQ ID NO: 125 | MGSSHHHHHHSSGLVPRGSHMVSDVPRDLEVVAATPTSLLISWDAPARRRRYYR |
| | ITYGETGGNSPVQEFTVPGSKSTATISGLKPGVDYTITVYAVTGWYWRSKPISI |
| | NYRTGGSGGSGGSELC |
| SEQ ID NO: 126 | MGSSHHHHHHSSGLVPRGSHMVSDVPRDLEVVAATPTSLLISWDAPAWYWRYYR |
| | ITYGETGGNSPVQEFTVPGSKSTATISGLKPGVDYTITVYAVTGRRRRSKPISI |
| | NYRTGGSGGSGGSELMEMEKEFEQIDKSGSWAAIYQDIRHEASDFPCRVAKLPK |
| | NKNRNRYRDVSPFDHSRIKLHQEDNDYINASLIKMEEAQRSYILTQGPLPNTCG |
| | HFWEMVWEQKSRGVVMLNRVMEKGSLKCAQYWPQKEEKEMIFEDTNLKLTLISE |
| | DIKSYYTVRQLELENLTTQETREILHFHYTTWPDFGVPESPASFLNFLFKVRES |
| | GSLSPEHGPVVVHCSAGIGRSGTFCLADTCLLLMDKRKDPSSVDIKKVLLEMRK |
| | FRMGLIQTADQLRFSYLAVIEGAKFIMGDSSVQDQWKELSHEDLEPPPEHIPPP |
| | PRPPKRILEPHN |
| SEQ ID NO: 127 | MGSSHHHHHHSSGLVPRGSHMVSDVPRDLEVVAATPTSLLISWDAPAWYWRYYR |
| | ITYGETGGNSPVQEFTVPGSKSTATISGLKPGVDYTITVYAVTGRRRRSKPISI |
| | NYRTGGSGGSGGSELGSVSSVPTKLEVVAATPTSLLISWDAPAVTVDYYVITYG |
| | ETGGNSPVQKFEVPGSKSTATISGLKPGVDYTITVYAWGWHGQVYYYMGSPISI |
| | NYRT |
| SEQ ID NO: 128 | MGSSHHHHHHSSGLVPRGSHMVSDVPRDLEVVAATPTSLLISWDAPAWYWRYYR |
| | ITYGETGGNSPVQEFTVPGSKSTATISGLKPGVDYTITVYAVTGRRRRSKPISI |
| | NYRTGGSGGSGGSELKTSNTIRVLLPNQEWTVVKVRNGMSLHDSLMKALKRHGL |
| | QPESSAVFRLLHEHKGKKARLDWNTDAASLIGEELQVOFLDHVPLTTHNFARKT |
| | FLKLGIHRD |
| SEQ ID NO: 129 | MSLNTSGLGASTMQISIGGAGGNNGLLGTHMPGTSSSPGLFQSGGDNGLGGHNA |
| | NSALGQQPIDRQTIEQMAQLLAELLKSLLDSGEKLGDNFGASADSASGTGQQDL |
| | MTQVLNGLAKSMLDDLLTKQDGGTSFSEDDSGPAKDGNANAGANDPSKNDPSKS |
| | QGPQSANKTGNVDDANNQDPMQALMQLLEDLVKLLKAALHMQQPGGNDKGNGVG |
| | GDSGQNDDSTSGIDSTSDSSDPMQQLLKMFSEIMQSLFGDEQDGIDSTSGSRFT |
| | RTGIGMKAGIQALNDIGTHSDSSTRSFVNKGDRAMAKEIGQFMDQYPEVFGKPQ |
| | YQKGPGQEVKTDDKSWAKALSKPDDDGMTPASMEQFNKAKGMIKSAMAGDTONG |
| | NLQARGAGGSSLGIDAMMAGDAINMALGKLGAA |
| SEQ ID NO: 130 | ATGAGTCTGAATACAAGTGGGCTGGGAGCGTCAACGATGCAAATTTCATCGGCG |
| | GTGCGGGCGGAAATAACGGGTTGCTGGGTACGCATATGCCCGGGACCTCGTCCT |
| | CGCCGGGTCTGTTCCAGTCCGGGGGGGACAACGGGCTTGGTGGTCATAATGCAA |
| | ATTCTGCGTTGGGGCAACAACCCATCGATCGGCAAACCAGAGCAAATGGCTCAA |
| | TTATTGGCGGAACTGTTAAAGTCACTGCTAGATAGTGGGGAAAAGCTCGGTGAC |
| | AACCGGCGCGTCTGCGGACAGCGCCTCGGGTACCGGACAGCAGGACCTGATGAC |
| | TCAGGTGCTCAATGGCCTGGCCAAGTCGATGCTCGATGATCTTCTGACCAAGCA |
| | GGATGGCGGGACCAGCTTCTCCGAAGACGATAGTGGGCCGGCGAAGGACGGCAA |
| | TGCCAACGCGGGCGCCAACGACCCGAGCAAGAACGACCCGAGCAAGAGCCAGGG |
| | TCCGCAGTCGGCCAACAAGACCGGCAACGTCGACGACGCCAACAACCAGGATCC |
| | GATGCAAGCGCTGATGCAGCTGCTGGAAGACCTGGTGAAGCTGCTGAAGGCGGC |
| | CCTGCACATGCAGCAGCCCGGCGGCAATGACAAGGGCAACGGCGTGGGCGGTGA |
| | TAGTGGGCAAAACGACGATTCCACCTCCGGCACAGATTCCACCTCAGACTCCAG |
| | CGACCCGATGCAGCAGCTGCTGAAGATGTTCAGCGAGATAATGCAAAGCCTGTT |
| | TGGTGATGAGCAAGATGGCACCGATAGTACTAGCGGCTCGAGGTTTACTCGTAC |
| | CGGTATCGGTATGAAAGCGGGCATTCAGGCGCTGAATGATATCGGTACGCACAG |
| | CGACAGCAACCCGTTCTTTCGTCAATAAAGGCGATCGGGCGATGGCGAAGGAAA |
| | TCGGTCAGTTCATGGACCAGTATCCTGAGGTGTTTGGCAAGCCGCAGTACCAGA |
| | AAGGCCCGGGTCAGGAGGTGAAAACCGATGACAAATCATGGGCAAAAGCACTGA |
| | GCAAGCCAGATGACGACGGAATGACACCAGCCAGTATGGAGCAGTTCAACAAAG |
| | CCAAGGGCATGATCAAAAGCGCCATGGCGGGTGATACCGGCAACGGCAACCTGC |
| | AGGCACGCGGTGCCGGTGGTTCTTCGCTGGGTATTGATGCCATGATGGCCGGTG |
| | ATGCCATTAACAATATGGCACTTGGCAAGCTGGGCGCGGCTTAA |
| SEQ ID NO: 131 | GGGGS |
| SEQ ID NO: 132 | GGGGS GGGGS GGGGS |
| SEQ ID NO: 133 | EAAAK |
| SEQ ID NO: 134 | AEAAAK EAAAK EAAAK EAAAKALEAEAAAK EAAAK EAAAK EAAAKA |
| SEQ ID NO: 135 | AEAAAKEAAAKA |
| SEQ ID NO: 136 | PAPAP |
| SEQ ID NO: 137 | VSQTSKLTRAETVFPDV |
| SEQ ID NO: 138 | PLGLWA |
| SEQ ID NO: 139 | RVLAEA |
| SEQ ID NO: 140 | EDVVCCSMSY |
| SEQ ID NO: 141 | GGIEGRGS |
| SEQ ID NO: 142 | TRHRQPRGWE |
| SEQ ID NO: 143 | AGNRVRRSVG |
| SEQ ID NO: 144 | RRRRRRRRR |
| SEQ ID NO: 145 | GFLG |
| SEQ ID NO: 146 | MQSLSLNSSSLQTPAMALVLVRPEAETTGSTSSKALQEVVVKLAEELMRNGQLD |
| | DSSPLGKLLAKSMAADGKAGGGIEDVIAALDKLIHEKLGDNFGASADSASGTGQ |
| | QDLMTQVLNGLAKSMLDDLLTKQDGGTSFSEDDMPMLNKIAQFMDDNPAQFPKP |
| | DSGSWVNELKEDNFLDGDETAAFRSALDIGQQLGNQQSDAGSLAGTGGGLGTPS |
| | SFSNNSSVMGDPLIDANTGPGDSGNTRGEAGQLIGELIDRGLQSVLAGGGLGTP |
| | VNTPQTGTSANGGQSAQDLDQLLGGLLLKGLEATLKDAGQTGTDVQSSAAQIAT |
| | LLVSTLLQGTRNQAAA |
| SEQ ID NO: 147 | ATAAATGAGCTCCAAAGCCTATCCTTAAATAGTTCATCTTTACAAAC |
| SEQ ID NO: 148 | AATAATCTCGAGTTAAGCCGCTGC |
| SEQ ID NO: 149 | TCCTGATGGGCAAGTGATTAC |
| SEQ ID NO: 150 | TTGTATGTGGTCTCGTGGATTC |
| SEQ ID NO: 151 | TGGACCCTGTAACTGGTTCTG |
| SEQ ID NO: 152 | GAACGTGACAATCAAGACGG |
| SEQ ID NO: 153 | ATGCCAAGGAAAGGGATTCTACA |
| SEQ ID NO: 154 | TGGGAGGTTTGGGCGAAGA |
| SEQ ID NO: 155 | ATGCAGAGTCTCAGTCTTAACAGCAGCTCGCTGCAAACCCCGGCAATGGCCCTT |
| | GTCCTGGTACGTCCTGAAGCCGAGACGACTGGCAGTACGTCGAGCAAGGCGCTT |
| | CAGGAAGTTGTCGTGAAGCTGGCCGAGGAACTGATGCGCAATGGTCAACTCGAC |
| | GACAGCTCGCCATTGGGAAAACTGTTGGCCAAGTCGATGGCCGCAGATGGCAAG |
| | GCGGGCGGCGGTATTGAGGATGTCATCGCTGCGCTGGACAAGCTGATCCATGAA |
| | AAGCTCGGTGACAACTTCGGCGCGTCTGCGGACAGCGCCTCGGGTACCGGACAG |
| | CAGGACCTGATGACTCAGGTGCTCAATGGCCTGGCCAAGTCGATGCTCGATGAT |
| | CTTCTGACCAAGCAGGATGGCGGGACAAGCTTCTCCGAAGACGATATGCCGATG |
| | CTGAACAAGATCGCGCAGTTCATGGATGACAATCCCGCACAGTTTCCCAAGCCG |
| | GACTCGGGCTCCTGGGTGAACGAACTCAAGGAAGACAACTTCCTTGATGGCGAC |
| | GAAACGGCTGCGTTCCGTTCGGCACTCGACATCATTGGCCAGCAACTGGGTAAT |
| | CAGCAGAGTGACGCTGGCAGTCTGGCAGGGACGGGTGGAGGTCTGGGCACTCCG |
| | AGCAGTTTTTCCAACAACTCGTCCGTGATGGGTGATCCGCTGATCGACGCCAAT |
| | ACCGGTCCCGGTGACAGCGGCAATACCCGTGGTGAAGCGGGGCAACTGATCGGC |
| | GAGCTTATCGACCGTGGCCTGCAATCGGTATTGGCCGGTGGTGGACTGGGCACA |
| | CCCGTAAACACCCCGCAGACCGGTACGTCGGCGAATGGCGGACAGTCCGCTCAG |
| | GATCTTGATCAGTTGCTGGGCGGCTTGCTGCTCAAGGGCCTGGAGGCAACGCTC |
| | AAGGATGCCGGGCAAACAGGCACCGACGTGCAGTCGAGCGCTGCGCAAATCGCC |
| | ACCTTGCTGGTCAGTACGCTGCTGCAAGGCACCCGCAACCAGGCTGCTGCCTGA |
| SEQ ID NO: 156 | APAYWWRYY |
| SEQ ID NO: 157 | TGRRRRSKPI |
| SEQ ID NO: 158 | APAWWARYY |
| SEQ ID NO: 159 | TGRRRRSKPI |
| SEQ ID NO: 160 | APAWWWRRYY |
| SEQ ID NO: 161 | TGRRASSKPI |
| SEQ ID NO: 162 | APAWWARYY |
| SEQ ID NO: 163 | TGWRRRRSKPI |
| SEQ ID NO: 164 | APAWWRRYY |
| SEQ ID NO: 165 | TGRRWWSKPI |
| SEQ ID NO: 166 | MGSSHHHHHHSSGLVPRGSHMVSDVPRDLEVVAATPTSLLISWDAPAWYWRYYR |
| | ITYGETGGNSPVQEFTVPGSKSTATISGLKPGVDYTITVYAVTGRRRRSKPISI |
| | NYRTGGSGGSGGSELSKGEELFTGVVPILVELDGDVNGHKFSVSGEGEGDATYG |
| | KLTLKFICTTGKLPVPWPTLVTTLTYGVQCFSRYPDHMKRHDFFKSAMPEGYVQ |
| | ERTIFFKDDGNYKTRAEVKFEGDTLVNRIELKGIDFKEDGNILGHKLEYNYNDH |
| | QVYIMADKQKNGIKANFKIRHNIEDGGVQLADHYQQNTPIGDGPVLLPDNHYLF |
| | TTSTLSKDPNEKRDHMVLLEFVTAAGITHGMDELYK |
| SEQ ID NO: 167 | MGSSHHHHHHSSGLVPRGSHMVSDVPRDLEVVAATPTSLLISWDAPAYWWRYYR |
| | ITYGETGGNSPVQEFTVPGSKSTATISGLKPGVDYTITVYAVTGRRRRSKPISI |
| | NYRTGGSGGSGGSELC |
| SEQ ID NO: 168 | MGSSHHHHHHSSGLVPRGSHMVSDVPRDLEVVAATPTSLLISWDAPAWWARYYR |
| | ITYGETGGNSPVQEFTVPGSKSTATISGLKPGVDYTITVYAVTGRRRRSKPISI |
| | NYRTGGSGGSGGSELC |
| SEQ ID NO: 169 | MGSSHHHHHHSSGLVPRGSHMVSDVPRDLEVVAATPTSLLISWDAPAWWWRRYY |
| | RITYGETGGNSPVQEFTVPGSKSTATISGLKPGVDYTITVYAVTGRRASSKPIS |
| | INYRTGGSGGSGGSELC |
| SEQ ID NO: 170 | MGSSHHHHHHSSGLVPRGSHMVSDVPRDLEVVAATPTSLLISWDAPAWWARYYR |
| | ITYGETGGNSPVQEFTVPGSKSTATISGLKPGVDYTITVYAVTGWRRRRSKPIS |
| | INYRTGGSGGSGGSELC |
| SEQ ID NO: 171 | MGSSHHHHHHSSGLVPRGSHMVSDVPRDLEVVAATPTSLLISWDAPAWWRRYYR |
| | ITYGETGGNSPVQEFTVPGSKSTATISGLKPGVDYTITVYAVTGRRWWSKPISI |
| | NYRTGGSGGSGGSELC |

1. A peptide, comprising:
a membrane translocation domain having one or more cell penetrating peptide motifs, and
a cargo moiety linked to the membrane translocation domain, wherein the cargo moiety comprises a plant bioactive moiety,
where at least one cell penetrating peptide motif is from 3 to 10 amino acid residues in length and has at least three arginine and/or lysine residues; or
where at least one cell penetrating peptide motif is from 3 to 10 amino acid residues in length and has at least two arginine and/or lysine residues and at least one other cell penetrating peptide motif is from 2 to 8 amino acid residues in length and has at least two hydrophobic residues.

2. The peptide of claim 1, wherein the membrane translocation domain is human fibronectin type III.

3. The peptide of claim 2, wherein the human fibronectin type III has 90% sequence similarity with SEQ ID NO: 118.

4. The peptide of claim 1, wherein the cell penetrating motif has from 3 to 10 adjacent arginine residues.

5. The peptide of claim 1, wherein the membrane translocation domain is human fibronectin type III having BC, DE, CD, and FG loops and one or more of the BC, DE, CD, or FG loops have cell penetrating peptide motifs.

6. (canceled)

7. (canceled)

8. (canceled)

9. The peptide of claim 1, wherein the cell penetrating peptide motif in the BC loop has from 2 to 8 amino acid residues and has at least two hydrophobic amino acid residues and the cell penetrating peptide motif in the FG loop has from 3 to 10 amino acid residues and has at least two adjacent arginine and/or lysine residues.

10. The peptide of claim 1, wherein the cell penetrating peptide motif in the FG loop has from 2 to 8 amino acid residues and has at least two hydrophobic amino acid residues and the cell penetrating peptide motif in the BC loop has from 3 to 10 amino acid residues and has at least two adjacent arginine and/or lysine residues.

11. The peptide of claim 1, wherein a second cell penetrating peptide motif is present and is WW, FF, WF, FW, WWW, FFF, WFW, FWF, WWF, WFF, FWW, FFW, WYW, WWH, YWW, or WYH.

12. The peptide of claim 1, wherein the cell penetrating peptide motif is RRRWWW (SEQ ID NO: 104) or WWWRRR (SEQ ID NO: 105).

13. The peptide of claim 1, wherein the peptide comprises TGRRRRWWWSKPI (SEQ ID NO: 111); APWWWRRRRYY (SEQ ID NO: 112); GGRRRRWWWVQE (SEQ ID NO: 113); APAWYWRYY (SEQ ID NO: 114); TGRRRRSKPI (SEQ ID NO: 115); APARRRRYY (SEQ ID NO: 116); TGWYWRSKPI (SEQ ID NO: 117); SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, or SEQ ID NO: 165.

14. The peptide of claim 1, wherein the peptide comprises SEQ ID NO.s: 119, 120, 121, 122, 123, 124, 125, 167, 168, 169, 170, or 171.

15. The peptide of claim 1, wherein the cargo moiety is linked to the membrane translocation domain at a N-terminus or C-terminus of the membrane translocation domain, or at a side chain within the membrane translocation domain.

16. (canceled)

17. The peptide of claim 1, wherein the peptide comprises SEQ ID NO.s: 126, 127, or 128.

18. The peptide of claim 1, wherein the plant bioactive moiety comprises synthetically derived or naturally occurring flagellins and flagellin-associated polypeptides (including those conserved among the Bacillus genera), thionins, harpin protein or polypeptide or harpin-like polypeptide, elongation factor Tu (EF-Tu), phytosulfokine (PSKα), root hair promoting polypeptide (RHPP), hypersensitive response elicitor proteins or polypeptides, or any combination thereof.

19. A composition for delivering a cargo moiety into a plant cell comprising SEQ ID NO: 122 covalently bound to a cargo moiety, wherein the cargo moiety comprises a plant bioactive moiety.

20. (canceled)

21. (canceled)

22. A method of delivering a cargo moiety into a plant, comprising contacting the plant with a peptide of claim 1.

23. A method of delivering a plant stimulant into a plant, comprising contacting the plant with a peptide of claim 1.

24. A method of delivering a plant activator into a plant, comprising contacting the plant with a peptide of claim 1.

25. A method to protect plants against biotic stress; stimulate seeds during germination; to protect plants against abiotic stress; to enhance growth, yield, health, longevity, productivity, and/or vigor of a plant; to provide multiple disease resistance to a plant; or any combination thereof, comprising contacting the plant with a peptide of claim 1.

26. A method of treating a plant that has a disease caused by a pathogenic agent, comprising contacting the plant with a peptide of claim 1.


SEQUENCE LISTING (171 sequences)

SEQ ID NO: 1 - protein, 5 aa, synthetic construct
  REGION 1..5: note = Description of sequence: CPP- cell penetrating peptide
  MOD_RES 2: note = naphthylalanine
FXRRR

SEQ ID NO: 2 - protein, 6 aa, synthetic construct
  REGION 1..6: note = Description of sequence: CPP- cell penetrating peptide
  MOD_RES 2: note = naphthylalanine
FXRRRC

SEQ ID NO: 3 - protein, 6 aa, synthetic construct
  REGION 1..6: note = Description of sequence: CPP- cell penetrating peptide
  MOD_RES 2: note = naphthylalanine
FXRRRU

SEQ ID NO: 4 - protein, 5 aa, synthetic construct
  REGION 1..5: note = Description of sequence: CPP- cell penetrating peptide
  MOD_RES 4: note = naphthylalanine
RRRXF

SEQ ID NO: 5 - protein, 6 aa, synthetic construct
  REGION 1..6: note = Description of sequence: CPP- cell penetrating peptide
  MOD_RES 5: note = naphthylalanine
RRRRXF

SEQ ID NO: 6 - protein, 6 aa, synthetic construct
  REGION 1..6: note = Description of sequence: CPP- cell penetrating peptide
  MOD_RES 2: note = naphthylalanine
FXRRRR

SEQ ID NO: 7 - protein, 6 aa, synthetic construct
  REGION 1..6: note = Description of sequence: CPP- cell penetrating peptide
  MOD_RES 2: note = D-naphthylalanine
  MOD_RES 3: note = D-arginine
  MOD_RES 5: note = D-arginine
FXRRRR

SEQ ID NO: 8 - protein, 6 aa, synthetic construct
  REGION 1..6: note = Description of sequence: CPP- cell penetrating peptide
  MOD_RES 2: note = D-naphthylalanine
  MOD_RES 3: note = D-arginine
  MOD_RES 5: note = D-arginine
FXRRRR

SEQ ID NO: 9 - protein, 6 aa, synthetic construct
  REGION 1..6: note = Description of sequence: CPP- cell penetrating peptide
  MOD_RES 2: note = naphthylalanine
FXRRRR

SEQ ID NO: 10 - protein, 6 aa, synthetic construct
  REGION 1..6: note = Description of sequence: CPP- cell penetrating peptide
  MOD_RES 2: note = naphthylalanine
  MOD_RES 1: note = D-phenylalanine
  MOD_RES 4: note = D-arginine
  MOD_RES 6: note = D-arginine
FXRRRR

SEQ ID NO: 11 - protein, 6 aa, synthetic construct
  REGION 1..6: note = Description of sequence: CPP- cell penetrating peptide
  MOD_RES 5: note = naphthylalanine
RRFRXR

SEQ ID NO: 12 - protein, 6 aa, synthetic construct
  REGION 1..6: note = Description of sequence: CPP- cell penetrating peptide
  MOD_RES 6: note = naphthylalanine
FRRRRX

SEQ ID NO: 13 - protein, 6 aa, synthetic construct
  REGION 1..6: note = Description of sequence: CPP- cell penetrating peptide
  MOD_RES 5: note = naphthylalanine
  MOD_RES 1: note = D-arginine
RRFRXR

SEQ ID NO: 14 - protein, 6 aa, synthetic construct
  REGION 1..6: note = Description of sequence: CPP- cell penetrating peptide
  MOD_RES 3: note = naphthylalanine
RRXFRR

SEQ ID NO: 15 - protein, 7 aa, synthetic construct
  REGION 1..7: note = Description of sequence: CPP- cell penetrating peptide
CRRRRFW

SEQ ID NO: 16 - protein, 7 aa, synthetic construct
  REGION 1..7: note = Description of sequence: CPP- cell penetrating peptide
  MOD_RES 3: note = naphthylalanine
  MOD_RES 2: note = D-phenylalanine
  MOD_RES 5: note = D-arginine
  MOD_RES 7: note = D-arginine
FFXRRRR

SEQ ID NO: 17 - protein, 7 aa, synthetic construct
  REGION 1..7: note = Description of sequence: CPP- cell penetrating peptide
  MOD_RES 3: note = naphthylalanine
FFXRRRR

SEQ ID NO: 18 - protein, 7 aa, synthetic construct
  REGION 1..7: note = Description of sequence: CPP- cell penetrating peptide
  MOD_RES 6: note = naphthylalanine
RFRFRXR

SEQ ID NO: 19 - protein, 7 aa, synthetic construct
  REGION 1..7: note = Description of sequence: CPP- cell penetrating peptide
URRRRFW

SEQ ID NO: 20 - protein, 7 aa, synthetic construct
  REGION 1..7: note = Description of sequence: CPP- cell penetrating peptide
CRRRRFW

SEQ ID NO: 21 - protein, 8 aa, synthetic construct
  REGION 1..8: note = Description of sequence: CPP- cell penetrating peptide
  MOD_RES 2: note = naphthylalanine
FXRRRRQK

SEQ ID NO: 22 - protein, 8 aa, synthetic construct
  REGION 1..8: note = Description of sequence: CPP- cell penetrating peptide
  MOD_RES 2: note = naphthylalanine
FXRRRRQC

SEQ ID NO: 23 - protein, 7 aa, synthetic construct
  REGION 1..7: note = Description of sequence: CPP- cell penetrating peptide
  MOD_RES 2: note = naphthylalanine
  MOD_RES 1: note = D-phenylalanine
  MOD_RES 4: note = D-arginine
  MOD_RES 6: note = D-arginine
FXRRRRR

SEQ ID NO: 24 - protein, 7 aa, synthetic construct
  REGION 1..7: note = Description of sequence: CPP- cell penetrating peptide
  MOD_RES 2: note = naphthylalanine
FXRRRRR

SEQ ID NO: 25 - protein, 9 aa, synthetic construct
  REGION 1..9: note = Description of sequence: CPP- cell penetrating peptide
  MOD_RES 5: note = naphthylalanine
  MOD_RES 8: note = Nle
RRRRXFDXC

SEQ ID NO: 26 - protein, 5 aa, synthetic construct
  REGION 1..5: note = Description of sequence: CPP- cell penetrating peptide
  MOD_RES 2: note = naphthylalanine
FXRRR

SEQ ID NO: 27 - protein, 5 aa, synthetic construct
  REGION 1..5: note = Description of sequence: CPP- cell penetrating peptide
FWRRR

SEQ ID NO: 28 - protein, 5 aa, synthetic construct
  REGION 1..5: note = Description of sequence: CPP- cell penetrating peptide
  MOD_RES 2: note = naphthylalanine
RRRXF

SEQ ID NO: 29 - protein, 5 aa, synthetic construct
  REGION 1..5: note = Description of sequence: CPP- cell penetrating peptide
RRRWF

SEQ ID NO: 30 - protein, 6 aa, synthetic construct
  REGION 1..6: note = Description of sequence: CPP- cell penetrating peptide
  MOD_RES 2: note = naphthylalanine
FXRRRR

SEQ ID NO: 31 - protein, 5 aa, synthetic construct
  REGION 1..5: note = Description of sequence: CPP- cell penetrating peptide
FFRRR

SEQ ID NO: 32 - protein, 5 aa, synthetic construct
  REGION 1..5: note = Description of sequence: CPP- cell penetrating peptide
  MOD_RES 3: note = D-arginine
  MOD_RES 5: note = D-arginine
FFRRR

SEQ ID NO: 33 - protein, 5 aa, synthetic construct
  REGION 1..5: note = Description of sequence: CPP- cell penetrating peptide
  MOD_RES 4: note = D-arginine
FFRRR

SEQ ID NO: 34 - protein, 5 aa, synthetic construct
  REGION 1..5: note = Description of sequence: CPP- cell penetrating peptide
FRFRR

SEQ ID NO: 35 - protein, 5 aa, synthetic construct
  REGION 1..5: note = Description of sequence: CPP- cell penetrating peptide
FRRFR

SEQ ID NO: 36 - protein, 5 aa, synthetic construct
  REGION 1..5: note = Description of sequence: CPP- cell penetrating peptide
FRRRF

SEQ ID NO: 37 - protein, 5 aa, synthetic construct
  REGION 1..5: note = Description of sequence: CPP- cell penetrating peptide
  MOD_RES 2: note = naphthylalanine
GXRRR

SEQ ID NO: 38 - protein, 5 aa, synthetic construct
  REGION 1..5: note = Description of sequence: CPP- cell penetrating peptide
FFFRA

SEQ ID NO: 39 - protein, 5 aa, synthetic construct
  REGION 1..5: note = Description of sequence: CPP- cell penetrating peptide
FFFRR

SEQ ID NO: 40 - protein, 6 aa, synthetic construct
  REGION 1..6: note = Description of sequence: CPP- cell penetrating peptide
FFRRRR

SEQ ID NO: 41 - protein, 6 aa, synthetic construct
  REGION 1..6: note = Description of sequence: CPP- cell penetrating peptide
FRRFRR

SEQ ID NO: 42 - protein, 6 aa, synthetic construct
  REGION 1..6: note = Description of sequence: CPP- cell penetrating peptide
FRRRFR

SEQ ID NO: 43 - protein, 6 aa, synthetic construct
  REGION 1..6: note = Description of sequence: CPP- cell penetrating peptide
RFFRRR

SEQ ID NO: 44 - protein, 6 aa, synthetic construct
  REGION 1..6: note = Description of sequence: CPP- cell penetrating peptide
RFRRFR

SEQ ID NO: 45 - protein, 6 aa, synthetic construct
  REGION 1..6: note = Description of sequence: CPP- cell penetrating peptide
FRFRRR

SEQ ID NO: 46 - protein, 6 aa, synthetic construct
  REGION 1..6: note = Description of sequence: CPP- cell penetrating peptide
FFFRRR

SEQ ID NO: 47 - protein, 6 aa, synthetic construct
  REGION 1..6: note = Description of sequence: CPP- cell penetrating peptide
FFRRRF

SEQ ID NO: 48 - protein, 6 aa, synthetic construct
  REGION 1..6: note = Description of sequence: CPP- cell penetrating peptide
FRFFRR

SEQ ID NO: 49 - protein, 6 aa, synthetic construct
  REGION 1..6: note = Description of sequence: CPP- cell penetrating peptide
RRFFFR

SEQ ID NO: 50 - protein, 6 aa, synthetic construct
  REGION 1..6: note = Description of sequence: CPP- cell penetrating peptide
FFRFRR

SEQ ID NO: 51 - protein, 6 aa, synthetic construct
  REGION 1..6: note = Description of sequence: CPP- cell penetrating peptide
FFRRFR

SEQ ID NO: 52 - protein, 6 aa, synthetic construct
  REGION 1..6: note = Description of sequence: CPP- cell penetrating peptide
FRRFFR

SEQ ID NO: 53 - protein, 6 aa, synthetic construct
  REGION 1..6: note = Description of sequence: CPP- cell penetrating peptide
FRRFRF

SEQ ID NO: 54 - protein, 6 aa, synthetic construct
  REGION 1..6: note = Description of sequence: CPP- cell penetrating peptide
FRFRFR

SEQ ID NO: 55 - protein, 6 aa, synthetic construct
  REGION 1..6: note = Description of sequence: CPP- cell penetrating peptide
RFFRFR

SEQ ID NO: 56 - protein, 6 aa, synthetic construct
  REGION 1..6: note = Description of sequence: CPP- cell penetrating peptide
  MOD_RES 2: note = naphthylalanine
GXRRRR

SEQ ID NO: 57 - protein, 7 aa, synthetic construct
  REGION 1..7: note = Description of sequence: CPP- cell penetrating peptide
FFFRRRR

SEQ ID NO: 58 - protein, 7 aa, synthetic construct
  REGION 1..7: note = Description of sequence: CPP- cell penetrating peptide
RFFRRRR

SEQ ID NO: 59 - protein, 7 aa, synthetic construct
  REGION 1..7: note = Description of sequence: CPP- cell penetrating peptide
RRFFRRR

SEQ ID NO: 60 - protein, 7 aa, synthetic construct
  REGION 1..7: note = Description of sequence: CPP- cell penetrating peptide
RFFFRRR

SEQ ID NO: 61 - protein, 7 aa, synthetic construct
  REGION 1..7: note = Description of sequence: CPP- cell penetrating peptide
RRFFFRR

SEQ ID NO: 62 - protein, 7 aa, synthetic construct
  REGION 1..7: note = Description of sequence: CPP- cell penetrating peptide
FFRRFRR

SEQ ID NO: 63 - protein, 7 aa, synthetic construct
  REGION 1..7: note = Description of sequence: CPP- cell penetrating peptide
FFRRRRF

SEQ ID NO: 64 - protein, 7 aa, synthetic construct
  REGION 1..7: note = Description of sequence: CPP- cell penetrating peptide
FRRFFRR

SEQ ID NO: 65 - protein, 8 aa, synthetic construct
  REGION 1..8: note = Description of sequence: CPP- cell penetrating peptide
FFFRRRRR

SEQ ID NO: 66 - protein, 9 aa, synthetic construct
  REGION 1..9: note = Description of sequence: CPP- cell penetrating peptide
FFFRRRRRR

SEQ ID NO: 67 - protein, 6 aa, synthetic construct
  REGION 1..6: note = Description of sequence: CPP- cell penetrating peptide
  MOD_RES 2: note = naphthylalanine
  MOD_RES 4: note = D-arginine
  MOD_RES 6: note = D-arginine
FXRRRR

SEQ ID NO: 68 - protein, 6 aa, synthetic construct
  REGION 1..6: note = Description of sequence: CPP- cell penetrating peptide
  MOD_RES 1: note = 4-fluorophenylalanine
  MOD_RES 2: note = 4-fluorophenylalanine
XXRRRR

SEQ ID NO: 69 - protein, 6 aa, synthetic construct
  REGION 1..6: note = Description of sequence: CPP- cell penetrating peptide
  MOD_RES 2: note = D-phenylalanine
  MOD_RES 5: note = D-arginine
FFFRRR

SEQ ID NO: 70 - protein, 6 aa, synthetic construct
  REGION 1..6: note = Description of sequence: CPP- cell penetrating peptide
  MOD_RES 1: note = D-phenylalanine
  MOD_RES 3: note = D-phenylalanine
  MOD_RES 4: note = D-arginine
  MOD_RES 6: note = D-arginine
FFFRRR

SEQ ID NO: 71 - protein, 6 aa, synthetic construct
  REGION 1..6: note = Description of sequence: CPP- cell penetrating peptide
  MOD_RES 1: note = D-phenylalanine
  MOD_RES 3: note = D-phenylalanine
  MOD_RES 5: note = D-arginine
FFFRRR

SEQ ID NO: 72 - protein, 6 aa, synthetic construct
  REGION 1..6: note = Description of sequence: CPP- cell penetrating peptide
  MOD_RES 2: note = D-phenylalanine
  MOD_RES 4: note = D-arginine
  MOD_RES 6: note = D-arginine
FFFRRR

SEQ ID NO: 73 - protein, 6 aa, synthetic construct
  REGION 1..6: note = Description of sequence: CPP- cell penetrating peptide
  MOD_RES 3: note = naphthylalanine
  MOD_RES 1: note = D-phenylalanine
  MOD_RES 4: note = D-arginine
  MOD_RES 6: note = D-arginine
FFXRRR

SEQ ID NO: 74 - protein, 6 aa, synthetic construct
  REGION 1..6: note = Description of sequence: CPP- cell penetrating peptide
  MOD_RES 2: note = naphthylalanine
  MOD_RES 1: note = D-phenylalanine
  MOD_RES 3: note = D-phenylalanine
  MOD_RES 4: note = D-arginine
  MOD_RES 6: note = D-arginine
FXFRRR

SEQ ID NO: 75 - protein, 6 aa, synthetic construct
  REGION 1..6: note = Description of sequence: CPP- cell penetrating peptide
  MOD_RES 1: note = naphthylalanine
  MOD_RES 3: note = D-phenylalanine
  MOD_RES 4: note = D-arginine
  MOD_RES 6: note = D-arginine
XFFRRR

SEQ ID NO: 76 - protein, 5 aa, synthetic construct
  REGION 1..5: note = Description of sequence: CPP- cell penetrating peptide
  MOD_RES 2: note = naphthylalanine
  MOD_RES 3: note = D-arginine
  MOD_RES 5: note = D-arginine
FXRRR

SEQ ID NO: 77 - protein, 5 aa, synthetic construct
  REGION 1..5: note = Description of sequence: CPP- cell penetrating peptide
  MOD_RES 2: note = naphthylalanine
  MOD_RES 1: note = D-phenylalanine
  MOD_RES 3: note = D-arginine
  MOD_RES 5: note = D-arginine
FXRRR

SEQ ID NO: 78 - protein, 8 aa, synthetic construct
  REGION 1..8: note = Description of sequence: CPP- cell penetrating peptide
  MOD_RES <1: note = acetylated
  MOD_RES 2: note = D-phenylalanine
  MOD_RES 5: note = D-arginine
  MOD_RES 7: note = D-arginine
KFFRRRRD

SEQ ID NO: 79 - protein, 8 aa, synthetic construct
  REGION 1..8: note = Description of sequence: CPP- cell penetrating peptide
  MOD_RES <1: note = acetylated
  MOD_RES 1: note = Dpr
  MOD_RES 2: note = D-phenylalanine
  MOD_RES 5: note = D-arginine
  MOD_RES 7: note = D-arginine
XFFRRRRD

SEQ ID NO: 80 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 5: note = D-arginine
  MOD_RES 6: note = D-arginine
  MOD_RES 7: note = D-glutamic acid
XXRERRE

SEQ ID NO: 81 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 5: note = D-arginine
  MOD_RES 6: note = D-arginine
  MOD_RES 7: note = D-glutamic acid
XXRRRRE

SEQ ID NO: 82 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 3: note = naphthylalanine
  MOD_RES 5: note = D-arginine
  MOD_RES 6: note = D-arginine
  MOD_RES 7: note = D-glutamic acid
XXXRRRE

SEQ ID NO: 83 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 3: note = naphthylalanine
  MOD_RES 5: note = D-arginine
  MOD_RES 6: note = D-arginine
XXXRRRE

SEQ ID NO: 84 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 5: note = D-arginine
  MOD_RES 6: note = D-arginine
  MOD_RES 7: note = D-glutamic acid
XXFRRRE

SEQ ID NO: 85 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 5: note = D-arginine
  MOD_RES 6: note = D-arginine
XXFRRRE

SEQ ID NO: 86 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 5: note = D-arginine
  MOD_RES 6: note = D-arginine
  MOD_RES 7: note = D-glutamic acid
XXFRRRE

SEQ ID NO: 87 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 5: note = D-arginine
  MOD_RES 6: note = D-arginine
XXFRRRE

SEQ ID NO: 88 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 3: note = D-naphthylalanine
  MOD_RES 5: note = D-arginine
  MOD_RES 6: note = D-arginine
XXXRRRE

SEQ ID NO: 89 - protein, 7 aa, synthetic construct
  REGION 1..7: note = Description of sequence: CPP- cell penetrating peptide
  MOD_RES 1: note = pipecolic acid
  MOD_RES 2: note = naphthylalanine
  MOD_RES 3: note = D-naphthylalanine
  MOD_RES 5: note = D-arginine
  MOD_RES 6: note = D-arginine
  MOD_RES 7: note = D-glutamic acid
XXXRRRE

SEQ ID NO: 90 - protein, 10 aa, synthetic construct
  REGION 1..10: note = Description of sequence: cell penetrating peptide
  MOD_RES 2: note = cyclohexylalanine
  MOD_RES 3: note = D-arginine
  MOD_RES 4: note = cyclohexylalanine
  MOD_RES 5: note = D-arginine
  MOD_RES 6: note = cyclohexylalanine
  MOD_RES 7: note = D-arginine
  MOD_RES 8: note = cyclohexylalanine
  MOD_RES 9: note = D-arginine
  CROSSLNK 1..10: note = Cyclic bond
PXRXRXRXRG

SEQ ID NO: 91 - protein, 14 aa, synthetic construct
  REGION 1..14: note = Description of sequence: cell penetrating peptide
  MOD_RES 3: note = D-Valine
  MOD_RES 5: note = L-difluorophosphonomethyl phenylalanine
  MOD_RES 7: note = Dpr
  MOD_RES 9: note = L-naphthylalanine
  MOD_RES 14: note = Dpr
  CROSSLNK 1..7: note = Cyclic bond
  CROSSLNK 1..13: note = Cyclic bond
  MOD_RES 1: note = trimesic acid
XSVPXHXFXRRRRX

SEQ ID NO: 92 - protein, 18 aa, synthetic construct
  REGION 1..18: note = Description of sequence: cell penetrating peptide
  MOD_RES 2: note = D-alanine
  MOD_RES 3: note = MeGly
  MOD_RES 5: note = pThr
  MOD_RES 6: note = pipecolic acid
  MOD_RES 7: note = naphthylalanine
  MOD_RES 10: note = D-alanine
  MOD_RES 11: note = Dpr
  MOD_RES 13: note = naphthylalanine
  MOD_RES 18: note = Dpr
  CROSSLNK 1..11: note = Cyclic bond
  CROSSLNK 1..17: note = Cyclic bond
  MOD_RES 1: note = trimesic acid
XAXDXXXRAAXFXRRRRX

SEQ ID NO: 93 - protein, 16 aa, synthetic construct
  REGION 1..16: note = Description of sequence: CPP- cell penetrating peptide
CRRSRRGCGRRSRRCG

SEQ ID NO: 94 - protein, 6 aa, synthetic construct
  REGION 1..6: note = Description of sequence: CPP- cell penetrating peptide
  MOD_RES 2: note = dodecanoyl
KXRRRR

SEQ ID NO: 95 - protein, 16 aa, synthetic construct
  REGION 1..16: note = Description of sequence: CPP- cell penetrating peptide
LKKLCKLLKKLCKLAG

SEQ ID NO: 96 - protein, 9 aa, synthetic construct
  REGION 1..9: note = Description of sequence: CPP- cell penetrating peptide
  CROSSLNK 5..9: note = Cyclic bond
RRRRKRRRE

SEQ ID NO: 97 - protein, 9 aa, synthetic construct
  REGION 1..9: note = Description of sequence: CPP- cell penetrating peptide
  CROSSLNK 4..9: note = Cyclic bond
RRRKRRRRE

SEQ ID NO: 98 - protein, 9 aa, synthetic construct
  REGION 1..9: note = Description of sequence: CPP- cell penetrating peptide
  CROSSLNK 3..9: note = Cyclic bond
RRKRRRRRE

SEQ ID NO: 99 - protein, 9 aa, synthetic construct
  REGION 1..9: note = Description of sequence: CPP- cell penetrating peptide
  CROSSLNK 2..9: note = Cyclic bond
RKRRRRRRE

SEQ ID NO: 100 - protein, 8 aa, synthetic construct
  REGION 1..8: note = Description of sequence: CPP- cell penetrating peptide
CRCRCRCR

SEQ ID NO: 101 - protein, 12 aa, synthetic construct
  REGION 1..12: note = Description of sequence: CPP- cell penetrating peptide
  MOD_RES 1: note = propargylglycine
  MOD_RES 12: note = L-6-azido-2-amino-hexanoic
  CROSSLNK 1..12: note = Cyclic bond
XLRKRLRKFRNX

SEQ ID NO: 102 - protein, 9 aa, synthetic construct
  REGION 1..9: note = Description of sequence: CPP- cell penetrating peptide
  MOD_RES 2: note = Dpr
  MOD_RES 3: note = Dpr
  MOD_RES 4: note = Dpr
  MOD_RES 5: note = D-phenylalanine
  MOD_RES 6: note = Dpr
  MOD_RES 7: note = Dpr
TXXXFLXXT

SEQ ID NO: 103 - protein, 9 aa, synthetic construct
  REGION 1..9: note = Description of sequence: CPP- cell penetrating peptide
  MOD_RES 2: note = L-2-amino-3-guanidinylpropionic acid
  MOD_RES 3: note = Dpr
  MOD_RES 4: note = Dpr
  MOD_RES 5: note = D-phenylalanine
  MOD_RES 6: note = Dpr
  MOD_RES 7: note = Dpr
TXXXFLXXT

SEQ ID NO: 104 - protein, 6 aa, synthetic construct
  REGION 1..6: note = Description of sequence: FG loop
RRRWWW

SEQ ID NO: 105 - protein, 6 aa, synthetic construct
  REGION 1..6: note = Description of sequence: BC loop
WWWRRR

SEQ ID NO: 106 - protein, 6 aa, synthetic construct
  REGION 1..6: note = Description of sequence: FG loop
GDSPAS

SEQ ID NO: 107 - protein, 9 aa, synthetic construct
  REGION 1..9: note = Description of sequence: flexible linker
GGSGGSGGS

SEQ ID NO: 108 - protein, 13 aa, synthetic construct
  REGION 1..13: note = Description of sequence: Protein-loop
TGRGDSPASSKPI

SEQ ID NO: 109 - protein, 9 aa, synthetic construct
  REGION 1..9: note = Description of sequence: Protein-loop
APAVTVRYY

SEQ ID NO: 110 - protein, 8 aa, synthetic construct
  REGION 1..8: note = Description of sequence: Protein-loop
GGNSPVQE

SEQ ID NO: 111 - protein, 13 aa, synthetic construct
  REGION 1..13: note = Description of sequence: Protein-loop
TGRRRRWWWSKPI

SEQ ID NO: 112 - protein, 11 aa, synthetic construct
  REGION 1..11: note = Description of sequence: Protein-loop
APWWWRRRRYY

SEQ ID NO: 113 - protein, 12 aa, synthetic construct
  REGION 1..12: note = Description of sequence: Protein-loop
GGRRRRWWWVQE

SEQ ID NO: 114 - protein, 9 aa, synthetic construct
  REGION 1..9: note = Description of sequence: Protein-loop
APAWYWRYY

SEQ ID NO: 115 - protein, 10 aa, synthetic construct
  REGION 1..10: note = Description of sequence: Protein-loop
TGRRRRSKPI

SEQ ID NO: 116 - protein, 9 aa, synthetic construct
  REGION 1..9: note = Description of sequence: Protein-loop
APARRRRYY

SEQ ID NO: 117 - protein, 10 aa, synthetic construct
  REGION 1..10: note = Description of sequence: Protein-loop
TGWYWRSKPI

SEQ ID NO: 118 - protein, 127 aa, synthetic construct
  REGION 1..127: note = Description of sequence: Membrane translocation domain (MTD)
MGSSHHHHHHSSGLVPRGSHMVSDVPRDLEVVAATPTSLLISWDAPAVTVRYYRITYGETGGNSPVQEFTVPGSKSTATISGLKPGVDYTITVYAVTGRGDSPASSKPISINYRTGGSGGSGGSELC

SEQ ID NO: 119 - protein, 127 aa, synthetic construct
  REGION 1..127: note = Description of sequence: Membrane translocation domain (MTD)
MGSSHHHHHHSSGLVPRGSHMVSDVPRDLEVVAATPTSLLISWDAPAVTVRYYRITYGETGGNSPVQEFTVPGSKSTATISGLKPGVDYTITVYAVTGRRRRWWWSKPISINYRTGGSGGSGGSELC

SEQ ID NO: 120 - protein, 129 aa, synthetic construct
  REGION 1..129: note = Description of sequence: Membrane translocation domain (MTD)
MGSSHHHHHHSSGLVPRGSHMVSDVPRDLEVVAATPTSLLISWDAPWWWRRRRYYRITYGETGGNSPVQEFTVPGSKSTATISGLKPGVDYTITVYAVTGRGDSPASSKPISINYRTGGSGGSGGSELC

SEQ ID NO: 121 - protein, 131 aa, synthetic construct
  REGION 1..131: note = Description of sequence: Membrane translocation domain (MTD)
MGSSHHHHHHSSGLVPRGSHMVSDVPRDLEVVAATPTSLLISWDAPAVTVRYYRITYGETGGRRRRWWWVQEFTVPGSKSTATISGLKPGVDYTITVYAVTGRGDSPASSKPISINYRTGGSGGSGGSELC

SEQ ID NO: 122 - protein, 124 aa, synthetic construct
  REGION 1..124: note = Description of sequence: Membrane translocation domain (MTD)
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELC

SEQ ID NO: 123 - protein, 127 aa, synthetic construct
  REGION 1..127: note = Description of sequence: Membrane translocation domain (MTD)
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRGDSPASSKPISINYRTGGSGGSGGSELC

SEQ ID NO: 124 - protein, 124 aa, synthetic construct
  REGION 1..124: note = Description of sequence: Membrane translocation domain (MTD)
MGSSHHHHHHSSGLVPRGSHMVSDVPRDLEVVAATPTSLLISWDAPAVTVRYYRITYGETGGNSPVQEFTVPGSKSTATISGLKPGVDYTITVYAVTGRRRRSKPISINYRTGGSGGSGGSELC

SEQ ID NO: 125 - protein, 124 aa, synthetic construct
  REGION 1..124: note = Description of sequence: Membrane translocation domain (MTD)
MGSSHHHHHHSSGLVPRGSHMVSDVPRDLEVVAATPTSLLISWDAPARRRRYYRITYGETGGNSPVQEFTVPGSKSTATISGLKPGVDYTITVYAVTGWYWRSKPISINYRTGGSGGSGGSELC

SEQ ID NO: 126 - protein, 444 aa, synthetic construct
  REGION 1..444: note = Description of sequence: membrane translocation domain cargo fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN

SEQ ID NO: 127 - protein, 220 aa, synthetic construct
  REGION 1..220: note = Description of sequence: membrane translocation domain cargo fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELGSVSSVPTKLEVVAATPTSLLISWDAPAVTVDYYVITYGETGGNSPVQKFEVPGSKSTATISGLKPGVDYTITVYAWGWHGQVYYYMGSPISINYRT

SEQ ID NO: 128 - protein, 225 aa, synthetic construct
  REGION 1..225: note = Description of sequence: membrane translocation domain cargo fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRD

SEQ ID NO: 129 - protein, 411 aa, synthetic construct
  REGION 1..411: note = Description of sequence: hairpin fragment
MSLNTSGLGASTMQISIGGAGGNNGLLGTHMPGTSSSPGLFQSGGDNGLGGHNANSALGQQPIDRQTIEQMAQLLAELLKSLLDSGEKLGDNFGASADSASGTGQQDLMTQVLNGLAKSMLDDLLTKQDGGTSFSEDDSGPAKDGNANAGANDPSKNDPSKSQGPQSANKTGNVDDANNQDPMQALMQLLEDLVKLLKAALHMQQPGGNDKGNGVGGDSGQNDDSTSGTDSTSDSSDPMQQLLKMFSEIMQSLFGDEQDGTDSTSGSRFTRTGIGMKAGIQALNDIGTHSDSSTRSFVNKGDRAMAKEIGQFMDQYPEVFGKPQYQKGPGQEVKTDDKSWAKALSKPDDDGMTPASMEQFNKAKGMIKSAMAGDTGNGNLQARGAGGSSLGIDAMMAGDAINMALGKLGAA

SEQ ID NO: 130 - DNA, 1232 bp, synthetic construct
  misc_feature 1..1232: note = Description of sequence: hairpin fragment
  source 1..1232: mol_type = other DNA
atgagtctgaatacaagtgggctgggagcgtcaacgatgcaaatttcatcggcggtgcgggcggaaataacgggttgctgggtacgcatatgcccgggacctcgtcctcgccgggtctgttccagtccgggggggacaacgggcttggtggtcataatgcaaattctgcgttggggcaacaacccatcgatcggcaaaccagagcaaatggctcaattattggcggaactgttaaagtcactgctagatagtggggaaaagctcggtgacaaccggcgcgtctgcggacagcgcctcgggtaccggacagcaggacctgatgactcaggtgctcaatggcctggccaagtcgatgctcgatgatcttctgaccaagcaggatggcgggaccagcttctccgaagacgatagtgggccggcgaaggacggcaatgccaacgcgggcgccaacgacccgagcaagaacgacccgagcaagagccagggtccgcagtcggccaacaagaccggcaacgtcgacgacgccaacaaccaggatccgatgcaagcgctgatgcagctgctggaagacctggtgaagctgctgaaggcggccctgcacatgcagcagcccggcggcaatgacaagggcaacggcgtgggcggtgatagtgggcaaaacgacgattccacctccggcacagattccacctcagactccagcgacccgatgcagcagctgctgaagatgttcagcgagataatgcaaagcctgtttggtgatgagcaagatggcaccgatagtactagcggctcgaggtttactcgtaccggtatcggtatgaaagcgggcattcaggcgctgaatgatatcggtacgcacagcgacagcaacccgttctttcgtcaataaaggcgatcgggcgatggcgaaggaaatcggtcagttcatggaccagtatcctgaggtgtttggcaagccgcagtaccagaaaggcccgggtcaggaggtgaaaaccgatgacaaatcatgggcaaaagcactgagcaagccagatgacgacggaatgacaccagccagtatggagcagttcaacaaagccaagggcatgatcaaaagcgccatggcgggtgataccggcaacggcaacctgcaggcacgcggtgccggtggttcttcgctgggtattgatgccatgatggccggtgatgccattaacaatatggcacttggcaagctgggcgcggcttaa

SEQ ID NO: 131 - protein, 5 aa, synthetic construct
  REGION 1..5: note = Description of sequence: flexible linker
GGGGS

SEQ ID NO: 132 - protein, 15 aa, synthetic construct
  REGION 1..15: note = Description of sequence: flexible linker
GGGGSGGGGSGGGGS

SEQ ID NO: 133 - protein, 5 aa, synthetic construct
  REGION 1..5: note = Description of sequence: rigid linker
EAAAK

SEQ ID NO: 134 - protein, 46 aa, synthetic construct
  REGION 1..46: note = Description of sequence: rigid linker
AEAAAKEAAAKEAAAKEAAAKALEAEAAAKEAAAKEAAAKEAAAKA

SEQ ID NO: 135 - protein, 12 aa, synthetic construct
  REGION 1..12: note = Description of sequence: rigid linker
AEAAAKEAAAKA

SEQ ID NO: 136 - protein, 5 aa, synthetic construct
  REGION 1..5: note = Description of sequence: rigid linker
PAPAP

SEQ ID NO: 137 - protein, 17 aa, synthetic construct
  REGION 1..17: note = Description of sequence: cleavable linker
VSQTSKLTRAETVFPDV

SEQ ID NO: 138 - protein, 6 aa, synthetic construct
  REGION 1..6: note = Description of sequence: cleavable linker
PLGLWA

SEQ ID NO: 139 - protein, 6 aa, synthetic construct
  REGION 1..6: note = Description of sequence: cleavable linker
RVLAEA

SEQ ID NO: 140 - protein, 10 aa, synthetic construct
  REGION 1..10: note = Description of sequence: cleavable linker
EDVVCCSMSY

SEQ ID NO: 141 - protein, 8 aa, synthetic construct
  REGION 1..8: note = Description of sequence: cleavable linker
GGIEGRGS

SEQ ID NO: 142 - protein, 10 aa, synthetic construct
  REGION 1..10: note = Description of sequence: cleavable linker
TRHRQPRGWE

SEQ ID NO: 143 - protein, 10 aa, synthetic construct
  REGION 1..10: note = Description of sequence: cleavable linker
AGNRVRRSVG

SEQ ID NO: 144 - protein, 9 aa, synthetic construct
  REGION 1..9: note = Description of sequence: cleavable linker
RRRRRRRRR

SEQ ID NO: 145 - protein, 4 aa, synthetic construct
  REGION 1..4: note = Description of sequence: cleavable linker
GFLG

SEQ ID NO: 146 - protein, 341 aa, Pseudomonas syringae
  REGION 1..341: note = Description of sequence: Pseudomonas syringae pv. glycinea strain A1 harpin Z (hrpZ) protein
MQSLSLNSSSLQTPAMALVLVRPEAETTGSTSSKALQEVVVKLAEELMRNGQLDDSSPLGKLLAKSMAADGKAGGGIEDVIAALDKLIHEKLGDNFGASADSASGTGQQDLMTQVLNGLAKSMLDDLLTKQDGGTSFSEDDMPMLNKIAQFMDDNPAQFPKPDSGSWVNELKEDNFLDGDETAAFRSALDIIGQQLGNQQSDAGSLAGTGGGLGTPSSFSNNSSVMGDPLIDANTGPGDSGNTRGEAGQLIGELIDRGLQSVLAGGGLGTPVNTPQTGTSANGGQSAQDLDQLLGGLLLKGLEATLKDAGQTGTDVQSSAAQIATLLVSTLLQGTRNQAAA

SEQ ID NO: 147 - DNA, 47 bp, synthetic construct
  misc_feature 1..47: note = Description of sequence: primer sequences
  source 1..47: mol_type = other DNA
ataaatgagctccaaagcctatccttaaatagttcatctttacaaac

SEQ ID NO: 148 - DNA, 24 bp, synthetic construct
  misc_feature 1..24: note = Description of sequence: primer sequences
  source 1..24: mol_type = other DNA
aataatctcgagttaagccgctgc

SEQ ID NO: 149 - DNA, 21 bp, synthetic construct
  misc_feature 1..21: note = Description of sequence: primer sequences
  source 1..21: mol_type = other DNA
tcctgatgggcaagtgattac

SEQ ID NO: 150 - DNA, 22 bp, synthetic construct
  misc_feature 1..22: note = Description of sequence: primer sequences
  source 1..22: mol_type = other DNA
ttgtatgtggtctcgtggattc

SEQ ID NO: 151 - DNA, 21 bp, synthetic construct
  misc_feature 1..21: note = Description of sequence: primer sequences
  source 1..21: mol_type = other DNA
tggaccctgtaactggttctg

SEQ ID NO: 152 - DNA, 20 bp, synthetic construct
  misc_feature 1..20: note = Description of sequence: primer sequences
  source 1..20: mol_type = other DNA
gaacgtgacaatcaagacgg

SEQ ID NO: 153 - DNA, 23 bp, synthetic construct
  misc_feature 1..23: note = Description of sequence: primer sequences
  source 1..23: mol_type = other DNA
atgccaaggaaagggattctaca

SEQ ID NO: 154 - DNA, 19 bp, synthetic construct
  misc_feature 1..19: note = Description of sequence: primer sequences
  source 1..19: mol_type = other DNA
tgggaggtttgggcgaaga

SEQ ID NO: 155 - DNA, 1026 bp, Pseudomonas syringae
  misc_feature 1..1026: note = Description of sequence: Pseudomonas syringae pv. glycinea strain A1 harpin Z (hrpZ) gene
  source 1..1026: mol_type = genomic DNA
atgcagagtctcagtcttaacagcagctcgctgcaaaccccggcaatggcccttgtcctggtacgtcctgaagccgagacgactggcagtacgtcgagcaaggcgcttcaggaagttgtcgtgaagctggccgaggaactgatgcgcaatggtcaactcgacgacagctcgccattgggaaaactgttggccaagtcgatggccgcagatggcaaggcgggcggcggtattgaggatgtcatcgctgcgctggacaagctgatccatgaaaagctcggtgacaacttcggcgcgtctgcggacagcgcctcgggtaccggacagcaggacctgatgactcaggtgctcaatggcctggccaagtcgatgctcgatgatcttctgaccaagcaggatggcgggacaagcttctccgaagacgatatgccgatgctgaacaagatcgcgcagttcatggatgacaatcccgcacagtttcccaagccggactcgggctcctgggtgaacgaactcaaggaagacaacttccttgatggcgacgaaacggctgcgttccgttcggcactcgacatcattggccagcaactgggtaatcagcagagtgacgctggcagtctggcagggacgggtggaggtctgggcactccgagcagtttttccaacaactcgtccgtgatgggtgatccgctgatcgacgccaataccggtcccggtgacagcggcaatacccgtggtgaagcggggcaactgatcggcgagcttatcgaccgtggcctgcaatcggtattggccggtggtggactgggcacacccgtaaacaccccgcagaccggtacgtcggcgaatggcggacagtccgctcaggatcttgatcagttgctgggcggcttgctgctcaagggcctggaggcaacgctcaaggatgccgggcaaacaggcaccgacgtgcagtcgagcgctgcgcaaatcgccaccttgctggtcagtacgctgctgcaaggcacccgcaaccaggctgctgcctga

SEQ ID NO: 156 - protein, 9 aa, synthetic construct
  REGION 1..9: note = Description of sequence: protein loop
APAYWWRYY

SEQ ID NO: 157 - protein, 10 aa, synthetic construct
  REGION 1..10: note = Description of sequence: protein loop
TGRRRRSKPI

SEQ ID NO: 158 - protein, 9 aa, synthetic construct
  REGION 1..9: note = Description of sequence: protein loop
APAWWARYY

SEQ ID NO: 159 - protein, 10 aa, synthetic construct
  REGION 1..10: note = Description of sequence: protein loop
TGRRRRSKPI

SEQ ID NO: 160 - protein, 10 aa, synthetic construct
  REGION 1..10: note = Description of sequence: protein loop
APAWWWRRYY

SEQ ID NO: 161 - protein, 10 aa, synthetic construct
  REGION 1..10: note = Description of sequence: protein loop
TGRRASSKPI

SEQ ID NO: 162 - protein, 9 aa, synthetic construct
  REGION 1..9: note = Description of sequence: protein loop
APAWWARYY

SEQ ID NO: 163 - protein, 11 aa, synthetic construct
  REGION 1..11: note = Description of sequence: protein loop
TGWRRRRSKPI

SEQ ID NO: 164 - protein, 9 aa, synthetic construct
  REGION 1..9: note = Description of sequence: protein loop
APAWWRRYY

SEQ ID NO: 165 - protein, 10 aa, synthetic construct
  REGION 1..10: note = Description of sequence: protein loop
TGRRWWSKPI

SEQ ID NO: 166 - protein, 360 aa, synthetic construct
  REGION 1..360: note = Description of sequence: membrane translocation domain cargo fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNDHQVYIMADKQKNGIKANFKIRHNIEDGGVQLADHYQQNTPIGDGPVLLPDNHYLFTTSTLSKDPNEKRDHMVLLEFVTAAGITHGMDELYK

SEQ ID NO: 167 - protein, 124 aa, synthetic construct
  REGION 1..124: note = Description of sequence: Membrane translocation domain (MTD)
MGSSHHHHHHSSGLVPRGSHMVSDVPRDLEVVAATPTSLLISWDAPAYWWRYYRITYGETGGNSPVQEFTVPGSKSTATISGLKPGVDYTITVYAVTGRRRRSKPISINYRTGGSGGSGGSELC

SEQ ID NO: 168 - protein, 124 aa, synthetic construct
  REGION 1..124: note = Description of sequence: Membrane translocation domain (MTD)
MGSSHHHHHHSSGLVPRGSHMVSDVPRDLEVVAATPTSLLISWDAPAWWARYYRITYGETGGNSPVQEFTVPGSKSTATISGLKPGVDYTITVYAVTGRRRRSKPISINYRTGGSGGSGGSELC

SEQ ID NO: 169 - protein, 125 aa, synthetic construct
  REGION 1..125: note = Description of sequence: Membrane translocation domain (MTD)
MGSSHHHHHHSSGLVPRGSHMVSDVPRDLEVVAATPTSLLISWDAPAWWWRRYYRITYGETGGNSPVQEFTVPGSKSTATISGLKPGVDYTITVYAVTGRRASSKPISINYRTGGSGGSGGSELC

SEQ ID NO: 170 - protein, 125 aa, synthetic construct
  REGION 1..125: note = Description of sequence: Membrane translocation domain (MTD)
MGSSHHHHHHSSGLVPRGSHMVSDVPRDLEVVAATPTSLLISWDAPAWWARYYRITYGETGGNSPVQEFTVPGSKSTATISGLKPGVDYTITVYAVTGWRRRRSKPISINYRTGGSGGSGGSELC

SEQ ID NO: 171 - protein, 124 aa, synthetic construct
  REGION 1..124: note = Description of sequence: Membrane translocation domain (MTD)
MGSSHHHHHHSSGLVPRGSHMVSDVPRDLEVVAATPTSLLISWDAPAWWRRYYRITYGETGGNSPVQEFTVPGSKSTATISGLKPGVDYTITVYAVTGRRWWSKPISINYRTGGSGGSGGSELC